(19) United States
(12) Patent Application Publication
(10) Pub. No.: US 2023/0348619 A1
(43) Pub. Date: Nov. 2, 2023

(54) Combination Of T-Cell Redirecting Multifunctional Antibodies With Immune Checkpoint Modulators And Uses Thereof

(71) Applicant: Lindis Biotech GmbH, Planegg (DE)

(72) Inventors: Horst LINDHOFER, Munchen (DE); Peter RUF, Schongeising (DE)

(21) Appl. No.: 18/070,965

(22) Filed: Nov. 29, 2022

Related U.S. Application Data
(63) Continuation of application No. 16/462,710, filed on May 21, 2019, now abandoned.

(30) Foreign Application Priority Data
Nov. 29, 2016 (WO) PCT/EP2016/079128

(51) Int. Cl. C07K 16/30; A61P 35/00; C07K 16/28; C07K 16/46

(52) U.S. Cl. CPC C07K 16/3084; A61P 35/00; C07K 16/2809; C07K 16/2818; C07K 16/468; A61K 2039/507

(57) ABSTRACT

The present invention provides a combination of (i) an immune checkpoint modulator and (ii) a T-cell redirecting multifunctional antibody, or an antigen binding fragment thereof, for use in therapeutic treatment of a cancer disease. The T-cell redirecting multifunctional antibody comprises (a) a specificity against a T cell surface antigen; (b) a specificity against a cancer- and/or tumor-associated antigen; and (c) a binding site for human FcRI, FcγRIIa and/or FcγRIII, wherein the antibody, or the antigen binding fragment thereof, binds with a higher affinity to human FcγRI, FcγRIIa and/or FcγRIII than to human FcγRIIb.

The present invention relates to the field of immunotherapy of cancer diseases, in particular to the application of T-cell redirecting multifunctional antibodies and immune checkpoint modulators in therapeutic/curative treatment of cancer diseases.

Immunotherapy in oncology is a steadily growing field. This is demonstrated by the recent approval of Yervoy® (Ipilimumab, Bristol Myers Squibb), Opdivo® (Nivolumab, Bristol Myers Squibb) and Keytruda® (Pembrolizumab, Merck). These monoclonal antibodies (mAbs) are directed either against the cytotoxic T-lymphocyte-associated protein 4 (CTLA-4) or the programmed cell death protein 1 (PD-1). A third heavily explored cancer target is the programmed death ligand 1 (PD-L1). All these targets have in common that they are negative key regulators for T-lymphocytes, so called inhibitory immune checkpoint molecules. Immune checkpoints are molecules in the immune system, in particular on certain immune cells, that need to be activated (stimulatory or costimulatory checkpoint molecules) or inactivated (inhibitory checkpoint molecules) to start an immune response. Many of the immune checkpoints are regulated by interactions between specific receptor and ligand pairs. Often cancers protect themselves from the immune system by using these checkpoints to avoid being attacked by the immune system.

The PD-1 receptor is expressed on the surface of activated T cells and other immune cells, such as B cells. Its ligands (PD-L1 and PD-L2) are expressed on the surface of antigen-presenting cells, such as dendritic cells or macrophages, and other immune cells. Binding of PD-L1 or PD-12 to PD1 triggers a signal in the T cell, which essentially switches the T cell off or inhibits it. Under non-pathological conditions, this interaction prevents T cells from attacking other cells in the body. However, cancer cells often take advantage of this system and express high levels of PD-L1 on their surface. Thereby, cancer cells are able to switch off T cells expressing PD-1 and, thus, to suppress the anticancer immune response. Inhibitors of PD1 and/or its ligands, such as inhibitory/antagonistic monoclonal antibodies directed to PD1 or to its ligands, can boost the immune response against cancer cells and are, thus, promising in treating cancers. Examples of inhibitory/antagonistic monoclonal antibodies against PD1, which are currently approved, include Opdivo® (Nivolumab; Bristol Myers Squibb) and Keytruda® (Pembrolizumab; Merck). Other inhibitors of the PD1 pathway, which are currently in clinical phase II and/or III include Pidilizumab (mAb inhibiting PD1; CureTech/Medivation), Durvalumab (mAb inhibiting PD-L1; MedImmune/AstraZeneca), Avelumab (mAb inhibiting PD-L1; Merck Serona/Pfizer) and Atezolimab (mAb inhibiting PD-L1; Roche).

Yervoy® (Ipilimumab; Bristol Myers Squibb), another approved immune checkpoint modulator, is an inhibitory/antagonistic monoclonal antibody against cytotoxic T-lymphocyte-associated antigen 4 (CTLA-4). CTLA4 is also expressed on the surface of activated T cells and its ligands are expressed on the surface of professional antigen-presenting cells. CTLA-4 is thought to regulate T-cell proliferation early in an immune response, primarily in lymph nodes and affects the functioning of regulatory T cells. Another inhibitor of CTLA-4, which is currently in clinical phase II, is, for example, Tremelimumab (MedImmune/AstraZeneca).

The binding of the natural ligands B7.1 and B7.2 to CTLA-4 and of PD-L1/PD-L2 to PD-1 on activated T-cells inhibits positive signals mediated by the T-cell receptor (TCR) or the costimulatory receptor CD28 and thereby leads to suppression of T-cell responses as a natural mechanism to circumvent immunological overreaction. Intensive research and clinical development revealed that the blocking of immune checkpoint molecules by mAbs leads to sustained T-cell activation that can be harnessed to combat cancer. Therefore, antibody-mediated blocking of immune checkpoints is an effective approach to boost tumor-reactive T-cell functions.

Since immune checkpoint inhibiting (blocking) antibodies act via a rather non-specific activation of the immune system there are numerous approaches to combine them with other cancer treatment regimens. In order to reduce the tumor load immune checkpoint inhibiting (blocking) antibodies were combined with chemotherapy. The disadvantage of such an approach is that strong chemotherapeutic agents negatively impact on the function of the immune system, reducing the efficacy of the immunotherapeutic drugs. Alternatively, immune checkpoint inhibiting (blocking) antibodies are combined with other immune checkpoint inhibitors. An example is the combination therapy of Yervoy® and Opdivo®, which was approved by the FDA in 2015 for the treatment of patients with BRAF V600 wild-type, unresectable or metastatic melanoma. In addition, a successful phase 1b study on the combination of Durvalumab and Tremelimumab in non-small cell lung cancer was recently reported (Antonia, Scott et al., 2016, Safety and antitumour activity of durvalumab plus tremelimumab in non-small cell lung cancer: a multicentre, phase 1b study; Lancet Oncol, 2016 Feb. 5. pii: S1470-2045(15)00544-6. doi: 10.1016/S1470-2045(15)00544-6. [Epub ahead of print]).

The disadvantage, however, is a significant increase in negative side effects (Tsai and Daud. Nivolumab plus Ipilimumab in the treatment of advanced melanoma. Journal of Hematology & Oncology (2015) 8:123). Moreover, the combination of checkpoint modulators with each other only targets endogenous tumor-specific immunity, in particular since no tumor-specific antigens are targeted.

In view of the above, there is a need for an improved immunotherapy for use in the treatment of a cancer disease. It is thus the object of the present invention to overcome the drawbacks of current immunotherapies for cancer outlined above and to provide a novel combination for use in the treatment of a cancer disease, which improves the survival of patients suffering from cancer, and which has a lower risk for side effects.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

Combination for Use in Therapeutic Treatment of a Cancer Disease

In a first aspect the present invention provides a combination of
(i) an immune checkpoint modulator and
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb;
for use in therapeutic treatment of a cancer disease.

The combination of T-cell redirecting multifunctional antibodies (trAbs) with immune checkpoint modulators represents a novel anti-cancer treatment approach that combines several unique and complementary immunotherapies:

Firstly, negative side effects are reduced. The blockade of inhibitory immune checkpoint molecules leads to a strong and non-specific activation of T cells which can cause severe autoimmune disorders, such as colitis, diarrhea, pneumonitis, hepatitis etc. The combination with T-cell redirecting multifunctional antibodies guides the activated T-cells away from the healthy tissue to bring them to the tumor site. Thereby, autoimmune reactions can be inhibited or reduced.

In addition, the Fc receptor binding site of T-cell redirecting multifunctional antibodies recruits and stimulates accessory cells such as dendritic cells (DCs) or macrophages via activating Fc receptors. These cells provide additional stimuli to T cells, take up tumor cell debris and present tumor-derived peptides to the immune system. Thus, T-cell redirecting multifunctional antibodies not only lead to T cell-dependent tumor destruction, but also induce a long-lasting tumor-specific immunologic memory.

Moreover, the therapeutic efficacy is improved by sustained T-cell activation. The present inventors have found that the activation of T-cells by T-cell redirecting multifunctional antibodies is accompanied by an increased expression of immune checkpoint molecules, which—in turn—downregulates the activated T cells. Thus, the combinatorial usage of checkpoint inhibitor blocking antibodies leads to a sustained and prolonged T-cell activation, which is advantageous for the direct anti-tumor effect of multifunctional T-cell redirecting antibodies.

Accordingly, as found by the present inventors, the combination for use according to the present invention mediates in particular sustained T-cell activation as compared to the T-cell activation induced by (i) the immune checkpoint modulator alone, and/or (ii) the T-cell redirecting multifunctional antibody, or the antigen binding fragment thereof, alone.

In summary, the combination of T-cell redirecting multifunctional antibodies with immune checkpoint modulators considerably augments the therapeutic anti-tumor efficacy of the single drugs (FIG. 1). Moreover, it can even reduce the substantial negative side effects of immune checkpoint modulators.

In the following, the components of the combination for use according to the present invention, i.e. the immune checkpoint modulator and the T-cell redirecting multifunctional antibody comprising a specificity against a T cell surface antigen, a specificity against a cancer- and/or tumor-associated antigen and a binding site for human FcγRI, FcγRIIa and/or FcγRIII, and preferred embodiments thereof, are described in detail. Moreover, also the use in therapeutic treatment of a cancer disease and preferred embodiments thereof, are described in detail below. It is understood that (i) a preferred embodiment of the combination for use according to the present invention comprises a preferred embodiment of the immune checkpoint modulator; (ii) a preferred embodiment of the combination for use according to the present invention comprises a preferred embodiment of the T-cell redirecting multifunctional antibody comprising a specificity against a T cell surface antigen, a specificity against a cancer- and/or tumor-associated antigen and a binding site for human FcγRI, FcγRIIa and/or FcγRIII; and (iii) a preferred embodiment of the combination for use according to the present invention comprises a preferred embodiment of the use in therapeutic treatment of a cancer disease. A more preferred embodiment of the combination for use according to the present invention comprises (i) a preferred embodiment of the immune checkpoint modulator and a preferred embodiment of the T-cell redirecting multifunctional antibody comprising a specificity against a T cell surface antigen, a specificity against a cancer- and/or tumor-associated antigen and a binding site for human FcγRI, FcγRIIa and/or FcγRIII; (ii) a preferred embodiment of the T-cell redirecting multifunctional antibody comprising a specificity against a T cell surface antigen, a specificity against a cancer- and/or tumor-associated antigen and a binding site for human FcγRI, FcγRIIa and/or FcγRIII and a preferred embodiment of the use in therapeutic treatment of a cancer disease; or (iii) a preferred embodiment of the immune checkpoint modulator and a preferred embodiment of the use in therapeutic treatment of a cancer disease. Most preferably, an embodiment of the combination for use according to the present invention comprises (i) a preferred embodiment of the immune checkpoint modulator; (ii) a preferred embodiment of the T-cell redirecting multifunctional antibody comprising a specificity against a T cell surface antigen, a specificity against a cancer- and/or tumor-associated antigen and a binding site for human FcγRI, FcγRIIa and/or FcγRIII; and (iii) a preferred embodiment of the use in therapeutic treatment of a cancer disease.

It is understood that it is generally preferred in the combination for use according to the present invention that the immune checkpoint modulator and the T-cell redirecting multifunctional antibody (or the antigen binding fragment thereof) are directed to distinct targets. In other words, the T-cell redirecting multifunctional antibody (or the antigen binding fragment thereof) does preferably not comprise any specificity or binding site targeting the same immune checkpoint molecule (or ligand thereof) as the immune checkpoint modulator of the combination for use according to the present invention. Again, in other words, the immune checkpoint modulator does preferably not modulate an immune checkpoint molecule (or ligand thereof), which is targeted by the T-cell redirecting multifunctional antibody (or the antigen binding fragment thereof) of the combination for use according to the present invention.

T-Cell Redirecting Multifunctional Antibody

As used herein (i.e., throughout the present application), the term “antibody” encompasses various forms of antibodies, preferably monoclonal antibodies including, but not being limited to, whole antibodies, antibody fragments, human antibodies, chimeric antibodies, recombinant antibodies, humanized antibodies, synthetic antibodies, chemically modified antibodies and genetically engineered antibodies (variant or mutant antibodies) as long as the characteristic properties according to the invention are retained. Preferred examples of antibodies include monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, antibody derivatives, antibody analogues and fragments thereof, respectively. Recombinant antibodies, in particular recombinant monoclonal antibodies, are more preferred. Moreover, it is also preferred that the antibody is a multichain antibody, i.e. an antibody comprising more than one chain, which is thus different from a single chain antibody. Furthermore, the antibody, or the antigen-binding fragment, may be entirely or partially of human origin or humanized. Humanization of antibodies is known in the art (see, for example, Shalaby et al., J. Exp. Med. 175 (1992), 217; Mocikat et al., Transplantation 57 (1994), 405). Preferably, at least the (six) CDRs (complementary-determining regions) and/or the framework regions, more preferably the variable regions, are of human origin and/or humanized. Unless otherwise indicated, the term “antibody” includes, in addition to antibodies comprising two full-length heavy chains and two full-length light chains, also derivatives, variants, and fragments thereof. In some instances an “antibody” may include fewer chains.

Preferably, the antibody, or the antigen binding fragment thereof, for use according to the present invention is a monoclonal antibody, or antigen binding fragment thereof. Herein, a “monoclonal” antibody (mAb or moAb) is understood as antibody made by identical immune cells that are all clones of a unique parent cell, in contrast to polyclonal antibodies which are made from several different immune cells. Generally, it is possible to produce a monoclonal antibody that specifically bind to a specific substance.

The term “human antibody”, as used herein, is intended to include antibodies having variable and constant regions derived from human immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Nat. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). The term “human antibody” as used herein also comprises such antibodies which are modified, e.g. in the variable region and/or in the Fc region, to generate the properties according to the invention.

As used herein, the term “recombinant antibody” is intended to include all antibodies, which do not occur in nature, in particular antibodies that are prepared, expressed, created or isolated by recombinant means, such as antibodies isolated from a host cell such as for example a CHO cell or from an animal (e.g. a mouse) or antibodies expressed using a recombinant expression vector transfected into a host cell. Such recombinant antibodies have variable and constant regions in a rearranged form as compared to naturally occurring antibodies.

As used herein, the terms “antigen binding fragment,” “fragment,” and “antibody fragment” are used interchangeably to refer to any fragment of an antibody of the invention that retains the specific binding activity of the antibody for use according to the invention, in particular the specificity against a T cell surface antigen, the specificity against a cancer- and/or tumor-associated antigen, and a binding site for human FcγRI, FcγRIIa and/or FcγRIII. Examples of antibody fragments include, but are not limited to, sc (single chain) antibody, scFv-Fc, scFv-CH3, scDiabody-CH3, Diabody-CH3, minibody, scFv-KIH, Fab-scFv-Fc, scDiabody-Fc, Diabody-Fc, and tandem scFv-1c (e.g., as described in Spiess C., Zhai Q. and Carter P. J. (2015) Molecular Immunology 67: 95-106). Fragments of the antibodies of the invention can be obtained from the antibodies by methods that include digestion with enzymes, such as pepsin or papain, and/or by cleavage of disulfide bonds by chemical reduction. Alternatively, fragments of antibodies can be obtained by cloning and expression of part of the sequences of the heavy and/or light chains. The invention also encompasses single-chain Fv fragments (scFv) including the CH3 region derived from the heavy and light chains of an antibody of the invention. For example, the invention includes a scFv-CH3 or a scFv-Fc comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention are typically multivalent and may be contained in a variety of structures as described above. For instance, scFv molecules may be synthesized to create a trivalent “triabody” or a tetravalent “tetrabody.” The scFv molecules preferably include a domain of the Fc region. Although the specification, including the claims, may, in some places, refer explicitly to antigen binding fragment(s), antibody fragment(s), variant(s) and/or derivative(s) of antibodies, it is understood that the term “antibody” or “antibody of the invention” includes all categories of antibodies, namely, antigen binding fragment(s), antibody fragment(s), variant(s) and derivative(s) of antibodies.

The antibody, or the fragment thereof, for use according to the present invention is a T-cell redirecting multifunctional antibody, or a fragment thereof.

As used herein, a “T-cell redirecting” antibody, or a fragment thereof, is an antibody, or a fragment thereof, which provides both, a specificity against a T cell surface antigen as well as a specificity against a cancer- and/or tumor-associated antigen. This enables the antibody, or the fragment thereof, to redirect T-cells to cancer cells. Thereby, “a specificity against a T cell surface antigen” means in particular that the antibody, or the antigen binding fragment thereof, for use according to the present invention comprises a paratope, which recognizes an epitope of a T cell surface antigen. In other words, the phrase “a specificity against a T cell surface antigen” means in particular that the antibody, or the antigen binding fragment thereof, for use according to the present invention comprises a binding site for a T cell surface antigen. Accordingly, “a specificity against a cancer- and/or tumor-associated antigen” means in particular that the antibody, or the antigen binding fragment thereof, for use according to the present invention comprises a paratope, which recognizes an epitope of a cancer- and/or tumor-associated antigen. In other words, the phrase “a specificity against a cancer- and/or tumor-associated antigen” means in particular that the antibody, or the antigen binding fragment thereof, for use according to the present invention comprises a binding site for a cancer- and/or tumor-associated antigen.

Importantly, in contrast to conventional (“ordinary”) antibodies exhibiting just one single specificity, T-cell redirecting antibodies are able to bind to at least two different epitopes, namely, one epitope on a cancer/tumor cell, and one epitope on a T-cell, thereby “redirecting” the T cell to the cancer/tumor cell, resulting in T-cell mediated cell killing. Accordingly, the T-cell redirecting antibodies for use according to the present invention exhibit T-cell redirecting properties, i.e. the antibody is typically capable of reactivating tumor-specific T cells being in the anergic state and/or direct T-cells to the desired antigen (as provided by a specificity against a cancer- and/or tumor-associated antigen of the antibody).

As used herein, a “multifunctional” antibody, or a fragment thereof, is an antibody, or a fragment thereof, which is capable of interacting with multiple distinct binding sites simultaneously. Since the antibody, or the fragment thereof, for use according to the present invention comprises (at least) (a) a specificity against a T cell surface antigen, (b) a specificity against a cancer- and/or tumor-associated antigen, and (c) a binding site for human FcγRI, FcγRIIa and/or FcγRIII, the “multifunctional” antibody, or the fragment thereof, is at least a “trifunctional” antibody, or a fragment thereof. “Trifunctional” means that the antibody, or the fragment thereof, is capable of interacting with three distinct binding sites simultaneously.

As described above, T-cell redirecting multifunctional antibodies comprise a tumor-associated antigen (TAA)-specific binding arm, a second binding arm specific for a T cell surface antigen, such as CD3 expressed on T cells, and a binding site for human FcγRI, FcγRIIa and/or FcγRIII that in particular preferentially binds to activating Fcγ receptors, such as FcγRI, FcγRIIa and/or FcγRIII, which are present on accessory cells such as macrophages, dendritic cells (DCs), or natural killer (NK) cells. Without being bound to any theory, the present inventors assume the following mode of action of T-cell redirecting multifunctional antibodies in tumor therapy (Lindhofer H, Hess J, Ruf P. Trifunctional Triomab® antibodies for cancer therapy. In: Kontermann R E (ed.), Bispecific antibodies. Springer, Berlin, 2011, p. 289-312): The first crucial step in this mode of action is thought to be redirection of T cells to the tumor via the bispecific antibody-mediated crosslink of a TAA with the T cell surface antigen, such as CD3. Antibody-mediated engagement of a T cell surface antigen, such as CD3, as a component of the T-cell receptor complex is a powerful first stimulus to activate T cells in a major histocompatibility complex (MHC)-independent manner, accompanied by TNF-α and IFN-g secretion. However, the physiological activation of T cells requires a second signal. Attracted by opsonized T cells and tumor cells as well as proinflammatory cytokines, FcγRI-, FcγRIIa- and/or FcγRIII-positive immune cells are additionally engaged via the FcγRI, FcγRIIa and/or FcγRIII binding site of the T-cell redirecting multifunctional antibodies. A cluster of different immune cell types is formed at the tumor cell.

This tri-cell complex formation consisting of tumor cells, T cells, and FcγRI-, FcRIIa- and/or FcγRIII-positive accessory immune cells suggests several important consequences: First, there is mutual stimulation of accessory immune cells and T cells. I-cell redirecting multifunctional antibody-triggered interaction of T cells and CD14-positive monocytes results in the upregulation of CD83, CD86, and CD40 (Riechelmann H, Wiesneth M, Schauwecker P, Reinhardt P, Gronau S, Schmitt A, Schroen C, Atz J, Schmitt M (2007) Adoptive therapy of head and neck squamous cell carcinoma with antibody coated immune cells: a pilot clinical trial. Cancer Immunol Immunother 56:1397-1406; Stanglmaier M, Faltin M, Ruf P, Bodenhausen A, Schroder P, Lindhofer H (2008) Bi20 (FBTA05), a novel trifunctional bispecific antibody (anti-CD20_anti-CD3), mediates efficient killing of B-cell lymphoma cells even with very low CD20 expression levels. Int J Cancer 123:1181-1189; Zeidler R, Mysliwietz I, Csanady M, Walz A, Ziegler I, Schmitt B, Wollenberg B, Lindhofer H (2000) The Fc-region of a new class of intact bispecific antibody mediates activation of accessory cells and NK cells and induces direct phagocytosis of tumour cells. Br J Cancer 83:261-266). Thus, T cells receive a second co-stimulatory signal in the form of CD40/CD40L or CD80-CD86/CD28 interaction. As a consequence, they are profoundly and physiologically activated, as characterized by high secretion of IL-2 and strong proliferation with detection of the proliferation marker Ki-67. Additionally, the T-cell activation markers CD25 and CD69 are upregulated (Riechelmann H, Wiesneth M, Schauwecker P, Reinhardt P, Gronau S, Schmitt A, Schroen C, Atz J, Schmitt M (2007) Adoptive therapy of head and neck squamous cell carcinoma with antibody coated immune cells: a pilot clinical trial. Cancer Immunol Immunother 56:1397-1406). Conversely, accessory immune cells are stimulated by interaction with T cells and the FcgR crosslink. This stimulation is manifested as high levels of proinflammatory cytokines such as IL-6 and IL-12 are measured, which are mainly secreted by accessory cells. Furthermore, the cross-talk between accessory and T cells is indicated by the release of Th1-biased cytokines, especially IL-2 and IFN-g. Finally, the targeted tumor cells are efficiently destroyed by the concerted attack of different types of immune effector cells, as shown in allogeneic settings as well as in autologous human ex vivo systems (Gronau S S, Schmitt M, Thess B, Reinhardt P, Wiesneth M, Schmitt A, Riechelmann H (2005) Trifunctional bispecific antibody-induced tumor cell lysis of squamous cell carcinomas of the upper aerodigestive tract. Head Neck 27:376-382). Necrotic and apoptotic tumor cells and particles are phagocytosed (Riesenberg R, Buchner A, Pohla H, Lindhofer H (2001) Lysis of prostate carcinoma cells by trifunctional bispecific antibodies (alpha EpCAM_alpha CD3). J Histochem Cytochem 49:911-917, Zeidler R, Mysliwietz J, Csanady M, Walz A, Ziegler I, Schmitt B, Wollenberg B, Lindhofer H (2000) The Fc-region of a new class of intact bispecific antibody mediates activation of accessory cells and NK cells and induces direct phagocytosis of tumour cells. Br J Cancer 83:261-266) and may be processed and presented by professional antigen-presenting cells in a stimulatory context, the ideal prerequisite for anti-tumor immunization.

As surprisingly found by the present inventors, a T-cell redirecting multifunctional antibody (or an antigen-binding fragment thereof) as defined herein induces increased expression of immune checkpoint molecules, such as CTLA-4 (cf. Example 2, FIG. 2). Accordingly, it is preferred in the combination for use according to the present invention that the T-cell redirecting multifunctional antibody (or the antigen-binding fragment thereof) induces increased expression of an immune checkpoint molecule, such as CTLA-4.

Preferably, the antibody, or the antigen binding fragment thereof, for use according to the present invention is a trifunctional antibody or a trifunctional antigen binding fragment thereof, in particular a bispecific trifunctional antibody or a bispecific, trifunctional antigen binding fragment thereof.

In the context of the present invention, “trifunctional” antibodies (trAb) are understood in particular as a specific class of bispecific antibodies recruiting and activating T cells and, in particular, accessory immune cells, such as macrophages, dendritic cells, natural killer (NK) cells, and/or other FcγRI-, FcγRIIa- and/or FcγRIII-expressing cells, simultaneously at the targeted cancer/tumor by, e.g. their FcγRI, FcγRIIa and/or FcγRIII binding site. Thus, trifunctional bispecific antibodies have two antigen-binding sites (i.e. two paratopes). Typically, these two antigen-binding sites (paratopes) allow the antibodies to bind to cancer/tumor cells (cancer/tumor cell surface antigens) and to T cells (T cell surface antigens). Simultaneously, e.g. via their Fc moiety, in particular their Fcγ receptor binding site, positive accessory cells are recruited, for example monocytes/macrophages, natural killer cells, dendritic cells or other Fcγ receptor expressing cells. The simultaneous activation of these different classes of effector cells results in efficient killing of the tumor cells by various mechanisms such as phagocytosis and perforin-mediated cytotoxicity. Typically, the net effect of a trifunctional antibody is linking T cells and, in particular, Fcγ receptor positive accessory cells to tumor cells, leading to the destruction of the tumor cells.

Trifunctional antibodies evoke the removal of tumor cells in particular by means of (i) antibody-dependent cell-mediated cytotoxicity, (ii) T-cell mediated cell killing, and (iii) induction of anti-tumor immunity. In contrast, only the first mode of action is actually executed by conventional (monoclonal and monospecific) antibodies. Moreover, in contrast to conventional antibodies, trifunctional antibodies have a higher cytotoxic potential and they even bind to antigens, which are expressed relatively weakly. Thus, trifunctional antibodies are at an equivalent dose more potent (more than 1000-fold) in eliminating tumor cells compared to conventional antibodies.

In general, the T-cell redirecting multifunctional antibody for use according to the present invention is a multispecific antibody. As used herein, the term “multispecific” refers to the ability to bind to at least two different epitopes, e.g. on different antigens, such as on a T cell surface antigen and on a cancer/tumor antigen. Thus, terms like “bispecific”, trispecific”, “tetraspecific” etc. refer to the number of different epitopes to which the antibody can bind to. For example, conventional monospecific IgG-type antibodies have two identical epitope binding sites (paratopes) and can, thus, only bind to identical epitopes (but not to different epitopes). A multispecific antibody, in contrast, has at least two different types of paratopes and can, thus, bind to at least two different epitopes. As used herein, “paratope” refers to an epitope-binding site of the antibody. Moreover, a single “specificity” may refer to one, two, three or more identical paratopes in a single antibody (the actual number of paratopes in one single antibody molecule is referred to as “valency”). For example, a single native IgG antibody is monospecific and bivalent, since it has two identical paratopes. Accordingly, a multispecific antibody comprises at least two (different) paratopes. Thus, the term “multispecific antibodies” refers to antibodies having more than one paratope and the ability to bind to two or more different epitopes. The term “multispecific antibodies” comprises in particular bispecific antibodies, but typically also protein, e.g. antibody, scaffolds, which bind in particular to three or more different epitopes, i.e. antibodies with three or more paratopes.

In particular, the multispecific antibody, or the antigen binding fragment thereof, may comprise two or more paratopes, wherein some paratopes may be identical so that all paratopes of the antibody belong to at least two different types of paratopes and, hence, the antibody has at least two specificities. For example, the multispecific antibody or antigen binding fragment thereof according to the present invention may comprise four paratopes, wherein each two paratopes are identical (i.e. have the same specificity) and, thus, the antibody or fragment thereof is bispecific and tetravalent (two identical paratopes for each of the two specificities). Thus, “one specificity” refers in particular to one or more paratopes exhibiting the same specificity (which typically means that such one or more paratopes are identical) and, thus, “two specificities” may be realized by two, three, four five, six or more paratopes as long as they refer to only two specificities. Most preferably a multispecific antibody comprises one single paratope for each (of the at least two) specificity, i.e. the multispecific antibody comprises in total at least two paratopes. For example, a bispecific antibody comprises one single paratope for each of the two specificities, i.e. the antibody comprises in total two paratopes. It is also preferred that the antibody comprises two (identical) paratopes for each of the two specificities, i.e. the antibody comprises in total four paratopes. Preferably the antibody comprises three (identical) paratopes for each of the two specificities, i.e. the antibody comprises in total six paratopes.

Preferably, the antibody, or the antigen binding fragment thereof, for use according to the present invention is a bispecific antibody or a bispecific antigen binding fragment thereof.

In the context of the present invention, bispecific antibodies (BiAbs) comprise (exactly) two specificities. They are the most preferred type of multispecific antibodies and antigen binding fragments thereof. A bispecific antibody in the context of the present invention may be of any bispecific antibody format comprising an FcγRI, FcγRIIa and/or FcγRIII binding site, e.g., as described in Spiess C., Zhai Q. and Carter P. J. (2015) Molecular Immunology 67: 95.106. For example, BiAbs may be whole antibodies, such as whole IgG-like molecules, or fragments thereof which are not whole antibodies but retain antibody properties. These may be small recombinant formats, e.g. as tandem single chain variable fragment molecules (taFvs), diabodies (Dbs), single chain diabodies (scDbs), and various other derivatives of these (cf. e.g. Byrne H. et al. (2013) Trends Biotech, 31 (11): 621-632 with FIG. 2 showing various bispecific antibody formats). Several BiAb formats can redirect effector cells against target cells that play key roles in disease processes. For example, several BiAb formats can retarget effector cells towards tumor cells and a variety of BiAb constructs were designed to retarget cells of the immune system, for example by binding to and triggering Fc receptors on the surface of effector cells or by binding to T cell receptor (TCR) complexes.

Preferably, the multispecific, in particular bispecific, antibody, or the antigen binding fragment thereof is at least bivalent, i.e. it has at least two paratopes. More preferably, the multispecific, in particular bispecific, antibody, or the antigen binding fragment thereof is bivalent, trivalent, tetravalent, or hexavalent. Even more preferably, the multispecific, in particular bispecific, antibody, or the antigen binding fragment thereof is bivalent or tetravalent. Most preferably, the antibody, or the antigen binding fragment thereof, for use according to the present invention is a bispecific, bivalent antibody, i.e. an antibody having two paratopes: one recognizing a T cell surface antigen and the other recognizing a cancer- and/or tumor-associated antigen.

In contrast to the terms multi“specific”, e.g. bispecific, trispecific, tetraspecific and the like, the terms multi“functional”, e.g. trifunctional and the like, refer to the number of distinct binding sites in a more general sense, i.e. they include any binding sites (not only those binding to epitopes). Therefore, for example an FcγRI, FcγRIIa and/or FcγRIII binding site “counts” for the category multi“functional”, e.g. trifunctional and the like, but not for the category multi“specific”, e.g. bispecific, trispecific, tetraspecific and the like. For example, a trifunctional antibody may be mono-, bi- or trispecific—but in the context of the present invention (wherein the antibody has two specificities and an FcγRI, FcγRIIa and/or FcγRIII binding site), a trifunctional antibody is typically bispecific.

As used herein, the term “antigen” refers to any structural substance which serves as a target for the receptors of an adaptive immune response, in particular as a target for antibodies, T cell receptors, and/or B cell receptors. An “epitope”, also known as “antigenic determinant”, is the part (or fragment) of an antigen that is recognized by the immune system, in particular by antibodies, T cell receptors, and/or B cell receptors. Thus, one antigen has at least one epitope, i.e. a single antigen has one or more epitopes. An antigen may be (i) a peptide, a polypeptide, or a protein, (ii) a polysaccharide, (iii) a lipid, (iv) a lipoprotein or a lipopeptide, (v) a glycolipid, (vi) a nucleic acid, or (vii) a small molecule drug or a toxin. Thus, an antigen may be a peptide, a protein, a polysaccharide, a lipid, a combination thereof including lipoproteins and glycolipids, a nucleic acid (e.g. DNA, siRNA, shRNA, antisense oligonucleotides, decoy DNA, plasmid), or a small molecule drug (e.g. cyclosporine A, paclitaxel, doxorubicin, methotrexate, 5-aminolevulinic acid), or any combination thereof. Preferably, the antigen is selected from (i) a peptide, a polypeptide, or a protein, (ii) a polysaccharide, (iii) a lipid, (iv) a lipoprotein or a lipopeptide and (v) a glycolipid; more preferably, the antigen is a peptide, a polypeptide, or a protein.

T-Cell Surface Antigen

As used herein, “(an epitope of) a T cell surface antigen” refers to (an epitope from) a T cell surface-associated antigen or a T cell surface-specific antigen (also known as “I cell surface markers”). These are in particular “CD” (cluster of differentiation) molecules specific for T cells. CD molecules are cell surface markers useful for the identification and characterization of leukocytes. The CD nomenclature was developed and is maintained through the HLDA (Human Leukocyte Differentiation Antigens) workshop started in 1982. Whether or not a certain CD molecule is found on T cells (and, thus, represents a T cell surface antigen in the context of the present invention) may be retrieved, for example, from a variety of sources known to the person skilled in the art, such as http://www.ebioscience.com/resources/human-cd-chart.htm, BD Bioscience's “Human and Mouse CD Marker Handbook” (retrievable at https://www.bdbiosciences.com/documents/cd_marker_handbook.pdt), or from www.hcdm.org. Accordingly, examples of T cell surface antigens include for example those (humani CD markers positively indicated for T cells in the BD Bioscience's “Human and Mouse CD Marker Handbook” (retrievable at https://www.bdbiosciences.com/documents/cd_marker_handbook.pdf) or in other sources of “CD marker charts”.

Preferably, the T cell surface antigen is selected from the group consisting of CD2, CD3, CD4, CD5, CD6, CD8, CD28, CD40L and CD44. This means that the antibody, or the antigen binding fragment thereof, for use according to the present invention comprises a paratope, which preferably recognizes (is able to bind to) an epitope of a T cell surface antigen selected from the group consisting of CD3, CD2, CD4, CD5, CD6, CD8, CD28. CD40L and/or CD44.

Said specificity preferably facilitates the recruitment of T cells. Therein, CD is the abbreviation for “cluster of differentiation” (cluster of designation or classification determinant) as described above. In general, this is known as a protocol used for the identification and investigation of cell surface molecules providing targets for immunophenotyping of cells. In terms of physiology, CD molecules can act in numerous ways, often acting as receptors or ligands (the molecule that activates a receptor) important to the cell. A signal cascade is usually initiated, altering the behavior of the cell (see cell signaling. Some CD proteins do not play a role in cell signaling, but have other functions, such as cell adhesion. At present, CD for humans is numbered up to 364. The present invention refers to T-cell associated CD molecules.

Preferably, the T-cell surface antigen, against which the T-cell redirecting multifunctional antibody or the antigen-binding fragment thereof comprises a specificity, is not CD28. More preferably, the T-cell redirecting multifunctional antibody or the antigen-binding fragment does not comprise any specificity/binding site for CD28.

More preferably, the T cell surface antigen is CD2 or CD3, most preferably the T cell surface antigen is CD3. This means that the antibody, or the antigen binding fragment thereof, for use according to the present invention comprises a paratope, which more preferably recognizes an epitope of CD2 or CD3, most preferably the antibody, or the antigen binding fragment thereof, for use according to the present invention comprises a paratope, which recognizes an epitope of CD3. The CD3 (cluster of differentiation 3) is a T-cell co-receptor that helps to activate cytotoxic T cells. CD3 typically consists of a protein complex and is composed of four distinct chains. In mammals, the complex contains a CD3γ chain, a CD3δ chain, and two CD3e chains. These chains associate with a molecule known as the T-cell receptor (TCR) and the ζ-chain (zeta-chain) to generate an activation signal in T lymphocytes. The ICR, ζ-chain, and CD3 molecules together constitute the TCR complex.

Cancer- and/or Tumor-Associated Antigen

As used herein, “(an epitope of) a cancer- and/or tumor-associated antigen” refers to (an epitope of) a cancer-associated antigen, a cancer-specific antigen, a tumor-associated antigen and/or a tumor-specific antigen. Such epitopes/antigens are typically specific for or associated with a certain kind of cancer/tumor. Suitable cancer/tumor epitopes and antigens can be retrieved for example from cancer/tumor epitope databases, e.g. from van der Bruggen P, Stroobant V, Vigneron N, Van den Eynde B. Peptide database: T cell-defined tumor antigens. Cancer Immun 2013; URL: http://www.cancerimmunity.org/peptide/, wherein human tumor antigens are classified into four major groups on the basis of their expression pattern, or from the database “Tantigen” (TANTIGEN version 1.0, Dec. 1, 2009; developed by Bioinformatics Core at Cancer Vaccine Center, Dana-Farber Cancer Institute; URL: http://cvc.dfci.harvard.edu/tadb/). Specific examples of cancer-related, in particular tumor-related, or tissue-specific antigens useful in the context of the present invention include, but are not limited to, the following antigens: Epha2, Epha4, PCDGF, HAAH, Mesothelin; EPCAM; NY-ESO-1, glycoprotein MUC1 and NIUC10 mucins p5 (especially mutated versions), EGFR; cancer antigen 125 (CA 125), the epithelial glycoprotein 40 (EGP40) (Kievit et al., 1997, Int. J. Cancer 71: 237-245), squamous cell carcinoma antigen (SCC) (Lozza et al., 1997 Anticancer Res. 17: 525-529), cathepsin E (Mota et al., 1997, Am. J Pathol. 150: 1223-1229), CDC27 (including the mutated form of the protein), antigens triosephosphate isomerase, 707-AP. A60 mycobacterial antigen (Macs et al., 1996, J. Cancer Res. Clin. Oncol. 122: 296-300), AFP, alpha(v)beta(3)-integrin, ART-4, ASC, BAGE, β-catenin/m, BCL-2, bcr-abl, bcr-abl p190, bcr-abl p210, BRCA-1, BRCA-2, CA 19-9 (Tolliver and O'Brien, 1997, South Med. J. 90: 89-90; Tsuruta at al., 1997 Urol. Int. 58: 20-24), CA125, CALLA, CAMEL, carbonic anhydrase, CAP-1, CASP-8, CDC27/m, CDK-4/m, CD1, CD2, CD4, CD6, CD7. CD8, CD11, CD13, CD14, CD19, CD20, CD21, CD22, CD23, CD24, CD30 CD33, CD37, CD38, CD40, CD41, CD44v3, CD44v6, CD47, CD52, CD138, CEA (Huang et al., Exper Rev. Vaccines (2002)1:49-63), c-erb-2, CT9, CT10, Cyp-B, Dek-cain, DAM-6 (MAGE-B2), DAM-10 (MAGE-B1), EphA2 (Zantek et al., Cell Growth Differ. (1999) 10:629-38; Carles-Kinch et al., Cancer Res. (2002) 62:2840-7), EphA4 (Cheng at al., 2002, Cytokine Growth Factor Rev. 13:75.85), tumor associated Thomsen-Friedenreich antigen (Dahlenborg et al., 1997, Int. J Cancer 70: 63-71), ELF2M, ETV6-AML1, G250, GAGE-1, GAGE-2, GAGF-3, GAGE-4, GAGE-5, GAGE-6, GAGE-7B, GAGE-8, GD1a, GD1b, GD2, GD3, GnT-V, GM1, GM2, GM3, gp100 (Zajac et al., 1997, Int. J Cancer 71: 491-496), GT1b, GT3, GQ1, HAGE, HER2/neu, HLA, HLA-DR, HLA-A*0201-R1701, HPV-E7, HSP-27, HSP-70, HSP70-2M, HSP-72, HSP-90, HST-2, hTERT, hTRT, iCE, inhibitors of apoptosis such as survivin, KH-1 adenocarcinoma antigen (Deshpande and Danishefsky, 1997, Nature 387: 164-166), KIAA0205, K-ras, LAGF, LAGE-1, LDLR/FUT, Lewis Y antigen, MAGE-1, MAGE-2, MAGE-3, MAGE-6, MAGE-A1, MAGE-A2, MAGE-A3, MAGE-A4, MAGE-A6, MAGE-A10, MAGE-A12, MAGE-B5, MAGE-B6, MAGE-C2, MAGE-C3, MAGE D, MART-1, MART-1/Melan-A (Kawakami and Rosenberg, 1997, Int. Rev. Immunol. 14: 173-192), MC1R, MCSP, MDM-2, MHCII, mTOR, Myosin/m, MUC1, MUC2, MUM-1, MUM-2, MUM-3, neo-polyA polymerase, NA88-A, NFX2, NY-ESO-1, NY-ESO-1a (CAG-3), PAGE-4, PAP, Proteinase 3 (Molidrem et al., Blood (1996) 88:2450-7; Molldrem et al., Blood (1997) 90:2529-34), P15, p53, p97, p190, Pgp, PIK3CA, Pm1/RARa, PRAME, proteoglycan, PSA, PSM, PSMA, RAGE, RAS, RCAS1, RU1, RU2, SAGE, SART 1, SART-2, SART-3, SP17, SPAS-1, SSX2, SSX4 TEL/AML1, TPVm, Tyrosinase, TARP, telomerase, TRP-1 (gp75), TRP-2, TRP-2/INT2, VEGF, WT-1, Wue antigen, cell surface targets GC182, GT468 or GT512, and alternatively translated NY-ESO-ORF2 and CAMEL proteins, derived from the NY-ESO-1 and LAGE-1 genes.

More preferably, the cancer- and/or tumor-associated antigen is selected from the group consisting of EpCAM, HER2/neu, CEA, MAGE, proteoglycan, VEGF, EGFR, mTOR, PIK3CA, RAS, alpha(v)beta(3)-integrin, HLA, HLA-DR, ASC, carbonic anhydrase, CD1, CD2, CD4, CD6, CD7, CD8, CD11, CD13, CD14, CD19, CD20, CD21, CD22, CD23, CD24, CD30 CD33, CD37, CD38, CD40, CD41, CD47, CD52, CD138, c-erb-2, CALLA, MHCII, CD44v3, CD44v6, p97, GM1, GM2, GM3, GD1a, GD1b, GD2, GD3, GT1b, GT3, GQ1, NY-ESO-1, NFX2, SSX2, SSX4, Trp2, gp100, tyrosinase, MUC-1, telomerase, survivin, p53, CA125, Wue antigen, Lewis Y antigen, HSP-27, HSP-70, HSP-72, HSP-90, Pgp, MCSP, EphA2 and cell surface targets GC182, GT468 or GT512. This means that the antibody, or the antigen binding fragment thereof, for use according to the present invention comprises a paratope, which preferably recognizes (is able to bind to) an epitope of a cancer- and/or tumor-associated antigen selected from the group consisting of EpCAM, HER2/neu, CEA, MAGE, proteoglycan, VEGF, EGFR, mTOR, PIK3CA, RAS, alpha(v)beta(3)-integrin, HLA, HLA-DR, ASC, carbonic anhydrase, CD1, CD2, CD4, CD6, CD7, CD8, CD11, CD13, CD14, CD19, CD20, CD21, CD22, CD23, CD24, CD30, CD33, CD37, CD38, CD40, CD41, CD47, CD52, CD138, c-erb-2, CALLA, MHCII, CD44v3, CD44v6, p97, GM1, GM2, GM3, GD1a, GD1b, GD2, GD3, GT1b, GT3, GQ1, NY-ESO-1, NFX2, SSX2, SSX4, Trp2, gp100, tyrosinase, MUC-1, telomerase, survivin, p53, CA125, Wue antigen, Lewis Y antigen, HSP-27, HSP-70, HSP-72, HSP-90, Pgp, MCSP, EphA2 and cell surface targets GC182, GT468 or GT512.

Particularly preferably, the cancer- and/or tumor-associated antigen is selected from the group consisting of EpCAM, HER2/neu, CEA, MAGE, VEGF, EGFR, mTOR, PIK3CA, RAS, GD2, CD19, CD20, CD30, CD33 and CD38, more preferably the cancer- and/or tumor-associated antigen is selected from the group consisting of EpCAM, HER2/neu, CEA, GD2, CD19, CD20, CD30, CD33 and CD38, even more preferably the cancer- and/or tumor-associated antigen is selected from the group consisting of EpCAM, HER2/neu, GD2 and CD20 and most preferably the cancer- and/or tumor-associated antigen is EpCAM. This means that the antibody, or the antigen binding fragment thereof, for use according to the present invention comprises a paratope, which preferably recognizes an epitope of EpCAM, HER2/neu, CEA, MAGE, VEGF, EGFR, mTOR, PIK3CA, RAS, GD2, CD19, CD20, CD30, CD33 and CD38; more preferably the antibody, or the antigen binding fragment thereof, for use according to the present invention comprises a paratope, which recognizes an epitope of EpCAM, HER2/neu, CEA, GD2, CD19, CD20 or CD33; even more preferably the antibody, or the antigen binding fragment thereof, for use according to the present invention comprises a paratope, which recognizes an epitope of EpCAM, HER2/neu, GD2 or CD20; and most preferably the antibody, or the antigen binding fragment thereof, for use according to the present invention comprises a paratope, which recognizes an epitope of EpCAM or GD2.

It is also preferred that the cancer- and/or tumor-associated antigen, against which the T-cell redirecting multifunctional antibody or the antigen-binding fragment thereof comprises a specificity, is not PD-L1. More preferably, the T-cell redirecting multifunctional antibody or the antigen-binding fragment does not comprise any specificity/binding site for PD-L1. In more general, it is even more preferred that the cancer- and/or tumor-associated antigen, against which the T-cell redirecting multifunctional antibody or the antigen-binding fragment thereof comprises a specificity, is not an immune checkpoint molecule and/or a ligand thereof. Most preferably, the T-cell redirecting multifunctional antibody or the antigen-binding fragment does not comprise any specificity/binding site for an immune checkpoint molecule and/or a ligand thereof.

Preferably, the cancer and/or tumor-associated antigen (or an epitope thereon, respectively) to be recognized by the antibody, or the antigen binding fragment thereof, for use according to the present invention is EpCAM. Preferably, the cancer and/or tumor-associated antigen (or an epitope thereon, respectively) to be recognized by the antibody, or the antigen binding fragment thereof, for use according to the present invention is GD2. Preferably, the cancer and/or tumor-associated antigen (or an epitope thereon, respectively) to be recognized by the antibody, or the antigen binding fragment thereof, for use according to the present invention is Her2/neu. Preferably, the cancer and/or tumor-associated antigen (or an epitope thereon, respectively) to be recognized by the antibody, or the antigen binding fragment thereof, for use according to the present invention is GD3. Preferably, the cancer and/or tumor-associated antigen (or an epitope thereon, respectively) to be recognized by the antibody, or the antigen binding fragment thereof, for use according to the present invention is CD20. Preferably, the cancer and/or tumor-associated antigen (or an epitope thereon, respectively) to be recognized by the antibody, or the antigen binding fragment thereof, for use according to the present invention is CD19. Preferably, the cancer and/or tumor-associated antigen (or an epitope thereon, respectively) to be recognized by the antibody, or the antigen binding fragment thereof, for use according to the present invention is CD30. Alternatively, the cancer and/or tumor-associated antigen (or an epitope thereon, respectively) to be recognized by the antibody, or the antigen binding fragment thereof, for use according to the present invention is CEA, MAGE, VFGF, FGFR, mTOR, PIK3CA or RAS.

Preferably, the antibody, or the antigen binding fragment thereof, for use according to the present invention binds (i) by its first specificity, e.g. by its first paratope, to an epitope of the T-cell surface antigen selected from the group consisting of CD2, CD3, CD4, CD5, CD6, CD8, CD28, CD40L and CD44, preferably CD2 or CD3, more preferably CD3; and, (ii) by its second specificity, e.g. by its second paratope, to a cancer and/or tumor-associated antigen preferably selected from the group consisting of the tumor antigens EpCAM, HER2/neu, CEA, MAGE, VEGF, EGFR, mTOR, PIK3CA, RAS, GD2, CD19, CD20, CD30, CD33 and CD38.

More preferably, the antibody, or the antigen binding fragment thereof, for use according to the present invention binds (i) by its first specificity, e.g. by its first paratope, to an epitope of the T-cell surface antigen selected from the group consisting of CD2, CD3, CD4, CD5, CD6, CD8, CD28, CD40L and CD44, preferably CD2 or CD3, more preferably CD3; and, (ii) by its second specificity, e.g. by its second paratope, to a cancer and/or tumor-associated antigen preferably selected from the group consisting of the tumor antigens EpCAM, HER2/neu, CEA, MAGE, VEGF, EGFR, mTOR, PIK3CA, RAS, GD2, CD19, CD20, CD30, CD33 and CD38.

The antibody, or the antigen binding fragment thereof, for use according to the present invention preferably binds by its first specificity, e.g. by its first paratope, to an epitope of the T-cell surface antigen, preferably CD3, and, by its second specificity, e.g. by its second paratope, to a cancer and/or tumor-associated antigen preferably selected from the group consisting of the tumor antigens EpCAM, HER2/neu, CEA, MAGE, VEGF, EGFR, mTOR, PIK3CA, RAS, GD2, CD19, CD20, CD30, CD33 and CD38 or to the gangliosides GM1, GM2, GM3, GD1a, GD1b, GD3, GT1b, GT3 or GQ1.

Preferably, the antibody, or the antigen binding fragment thereof, for use according to the present invention comprises a first specificity against CD3 and a second specificity against a cancer- and/or tumor-associated antigen selected from the group consisting of EpCAM, HER2/neu, CEA, GD2, CD19, CD20 and CD33.

It is also preferred that the cancer- and/or tumor-associated antigen, against which the T-cell redirecting multifunctional antibody or the antigen-binding fragment thereof comprises a specificity, is not CD19. More preferably, the T-cell redirecting multifunctional antibody or the antigen-binding fragment does not comprise any specificity/binding site for CD19.

It is also preferred that the cancer- and/or tumor-associated antigen, against which the T-cell redirecting multifunctional antibody or the antigen-binding fragment thereof comprises a specificity, is not CD20. More preferably, the T-cell redirecting multifunctional antibody or the antigen-binding fragment does not comprise any specificity/binding site for CD20.

It is also preferred that the cancer- and/or tumor-associated antigen, against which the T-cell redirecting multifunctional antibody or the antigen-binding fragment thereof comprises a specificity, is not CEA. More preferably, the T-cell redirecting multifunctional antibody or the antigen-binding fragment does not comprise any specificity/binding site for CEA.

Preferably, the antibody, or the antigen binding fragment thereof, for use according to the present invention may comprise one specificity, preferably one paratope, against CD3 and one specificity, preferably one paratope, against EpCAM (anti-CD3×anti-EpCAM). Preferably, the antibody, or the antigen binding fragment thereof, for use according to the present invention may comprise one specificity, preferably one paratope, against CD3 and one specificity, preferably one paratope, against GD2 (anti-CD3×anti-GD2). Preferably, the antibody, or the antigen binding fragment thereof, for use according to the present invention may comprise one specificity, preferably one paratope, against CD3 and one specificity, preferably one paratope, against Her2/neu (anti-CD3×anti-Her2/neu). Preferably, the antibody, or the antigen binding fragment thereof, for use according to the present invention may comprise one specificity, preferably one paratope, against CD3 and one specificity, preferably one paratope, against GD3 (anti-CD3×anti-GD3). Preferably, the antibody, or the antigen binding fragment thereof, for use according to the present invention may comprise one specificity, preferably one paratope, against CD3 and one specificity, preferably one paratope, against CD20 (anti-CD3×anti-CD20). Preferably, the antibody, or the antigen binding fragment thereof, for use according to the present invention may comprise one specificity, preferably one paratope, against CD3 and one specificity, preferably one paratope, against CD19 (anti-CD3×anti-CD19). Preferably, the antibody, or the antigen binding fragment thereof, for use according to the present invention may comprise one specificity, preferably one paratope, against CD3 and one specificity, preferably one paratope, against CEA (anti-CD3×anti-CEA). Preferably, the antibody, or the antigen binding fragment thereof, for use according to the present invention may comprise one specificity, preferably one paratope, against CD3 and one specificity, preferably one paratope, against MAGE (anti-CD3×anti-MAGE). Preferably, the antibody, or the antigen binding fragment thereof, for use according to the present invention may comprise one specificity, preferably one paratope, against CD3 and one specificity, preferably one paratope, against VEGF (anti-CD3×anti-VEGF). Preferably, the antibody, or the antigen binding fragment thereof, for use according to the present invention may comprise one specificity, preferably one paratope, against CD3 and one specificity, preferably one paratope, against EGFR (anti-CD3×anti-EGFR). Preferably, the antibody, or the antigen binding fragment thereof, for use according to the present invention may comprise one specificity, preferably one paratope, against CD3 and one specificity, preferably one paratope, against mTOR (anti-CD3×anti-mTOR). Preferably, the antibody, or the antigen binding fragment thereof, for use according to the present invention may comprise one specificity, preferably one paratope, against CD3 and one specificity, preferably one paratope, against PIK3CA (anti-CD3×anti-PIK3CA). Preferably, the antibody, or the antigen binding fragment thereof, for use according to the present invention may comprise one specificity, preferably one paratope, against CD3 and one specificity, preferably one paratope, against RAS (anti-CD3×anti-RAS).

Alternatively, the antibody, or the antigen binding fragment thereof, for use according to the present invention may comprise one specificity, preferably one paratope, against CD3 and one specificity, preferably one paratope, against CD30 (anti-CD3×anti-CD30). Preferably, the antibody, or the antigen binding fragment thereof, for use according to the present invention may comprise one specificity, preferably one paratope, against CD3 and one specificity, preferably one paratope, against CD33 (anti-CD3×anti-CD33). Preferably, the antibody, or the antigen binding fragment thereof, for use according to the present invention may comprise one specificity, preferably one paratope, against CD3 and one specificity, preferably one paratope, against an arboviral E protein epitope (anti-CD3×anti-arboviral E protein).

Preferably, the antibody, or the antigen binding fragment thereof, for use according to the present invention comprises two specificities selected from anti-EpCAM×anti-CD3, anti-GD2×anti-CD3, anti-CD20×anti-CD3, anti-HER2/neu×anti-CD3, and anti-CD19×anti-CD3. More preferably, the antibody, or the antigen binding fragment thereof, for use according to the present invention comprises two specificities selected from anti-EpCAM×anti-CD3, anti-GD2×anti-CD3, and anti-HER2/neu×anti-CD3. Even more preferably, the antibody, or the antigen binding fragment thereof, for use according to the present invention comprises two specificities selected from anti-FpCAM×anti-CD3 and anti-GD2×anti-CD3.

Binding Site for Human FcγRI, FcγRIIa and/or FcγRIII

The antibody, or the antigen-binding fragment thereof, for use according to the present invention comprises a binding site for human FcγRI, FcγRIIa and/or FcγRIII. More preferably, the antibody, or the antigen-binding fragment thereof, for use according to the present invention comprises a binding site for human FcγRIIa. The binding site for human FcγRI, FcγRIIa and/or FcγRIII, for example the Fc region, enables the multifunctional antibody to additionally recruit cells expressing FcγRI. FcγRIIa and/or FcγRIII, such as FcγRI, FcγRIIa and/or FcγRIII positive accessory cells, for example macrophages, dendritic cells, natural killer (NK) cells, and other FcγRI, FcγRIIa and/or FcγRIII expressing cells. Since multifunctional antibodies are at least bispecific (or multispecific) antibodies, they are preferably able to recruit and activate (i) T cells and (ii) FcγRI, FcγRIIa and/or FcγRIII expressing cells, such as accessory immune cells, for example monocytes/macrophages, natural killer cells, dendritic cells or other FcγRI, FcγRIIa and/or FcγRIII expressing cells, simultaneously at the (iii) targeted cancer/tumor cells. The simultaneous activation of these different classes of effector cells results in efficient killing of the tumor cells by various mechanisms such as, for example, phagocytosis and perforin-mediated cytotoxicity. Typically, the net effect of a preferred multifunctional antibody, which comprises an FcγRI, FcγRIIa and/or FcγRIII binding site, is linking T cells and Fc receptor positive cells to target cells, e.g. tumor cells, leading to the destruction of the tumor cells. Multifunctional antibodies evoke the removal of tumor cells by means of (i) antibody-dependent cell-mediated cytotoxicity, (ii) T-cell mediated cell killing, and (iii) induction of anti-tumor immunity.

In general, Fc gamma receptors (FcγR) are a family of Fc receptors for IgG. All of the Fcγ receptors (FcγR) belong to the immunoglobulin superfamily and are the most important Fc receptors for inducing phagocytosis of opsonized (marked) microbes. This family includes several members: FcγRI (CD64), FcγRIIa (CD32a), FcγRIIb (CD32b), FcγRIIIa (CD16a), and FcγRIIIb (CD16b) in humans and FcγRI, FcγRIIb, FcγRIII, and FcγRIV in mice. The complexity in the FcγR family is mirrored by the presence of four different IgG subclasses in humans (IgG1-IgG4) and mice (IgG1, IgG2a, IgG2b and IgG3), which bind with varying affinity and specificity to different Fcγ receptors (for review see Nimmerjahn F. and Ravetch J. V., 2008, Fcγ receptors as regulators of immune responses, Nat Rev Immunol 8: 34-47). Traditionally, FcγR families are categorized according to the level of the receptor's affinity for specific IgG subclasses and the type of signaling pathway that it triggers, i.e. whether it is inhibitory or activating. FcγRIIb is conserved in mice and humans and is the only known inhibitory FcγR; it transmits inhibitory signals through an immunoreceptor tyrosine-based inhibitory motif (ITIM) contained in its cytoplasmic region. All other FcγR, with the exception of the human GPI-anchored FcγRIIIb, activate signaling pathways through ITAMs contained in their cytoplasmic regions. FcγRIa is the only known high-affinity FcγR in mice and humans. All other FcγR have a 100-1000-fold lower affinity in the low to medium micromolar range and show a broader IgG subclass specificity. The inhibitory FcγRIIb is the most broadly expressed FcγR, and is present on virtually all leukocytes with the exception of NK cells and T cells. Finally, it has been demonstrated that the activity of IgG1 is negatively regulated by the inhibitory FcγRIIb. Accordingly, it is assumed that the inhibitory FcγRIIb exerts a “regulatory” function on IgG responses.

Human FcγRIIa (immunoglobulin G (IgG) Fc receptor IIa; CD32a) is a low affinity receptor for IgG and is expressed on macrophages, neutrophils, eosinophils, platelets and dendritic cells. FcγRIIa delivers an activating signal upon ligand binding, and results in the initiation of inflammatory responses including cytolysis, phagocytosis, degranulation and cytokine production. Two genetically determined structurally different allotypes of human FcγRIIa are known: the products of the FcγRIIa-R131 and FcγRIIa-H131 alleles. FcγRIIa responses can be modulated by signals from the coexpressed inhibitory receptors such as FcγRIIb (CD32b), and the strength of the signal is dependent on the ratio of expression of the activating and inhibitory receptors.

The antibody, or the antigen-binding fragment thereof, for use according to the present invention comprises a binding site for human FcγRI, FcγRIIa and/or FcγRIII, wherein the antibody, or the antigen-binding fragment thereof, binds with a higher affinity to human FcγRI, FcγRIIa and/or FcγRIII than to human FcγRIIb. This means that, if the antibody, or the antigen-binding fragment thereof, binds to only one of FcγRI, FcγRIIa and/or FcγRIII (i.e., (i) the antibody, or the antigen-binding fragment thereof, binds to FcγRI, but not to FcγRIIa or FcγRIII; (ii) the antibody, or the antigen-binding fragment thereof, binds to FcγRIIa, but not to FcγRI or FcγRIII; or (iii) the antibody, or the antigen-binding fragment thereof, binds to FcγRIII, but not to Fc-RI or FcγRIIa), the antibody, or the antigen-binding fragment thereof, binds with a higher affinity to that one of human FcγRI, FcγRIIIa and/or FcγRIII than to human FcγRIIb. However, if the antibody, or the antigen-binding fragment thereof, binds to more than one of FcγRI, FcγRIIa and/or FcγRIII (i.e., (i) the antibody, or the antigen-binding fragment thereof, binds to FcγRI and FcγRIIa, but not to FcγRIII; (ii) the antibody, or the antigen-binding fragment thereof, binds to FcγRIIa and FcγRIII, but not to FcγRI; (iii) the antibody, or the antigen-binding fragment thereof, binds to FcγRI and FcγRIII, but not to FcγRIIa; or (iv) the antibody, or the antigen-binding fragment thereof, binds to FcγRI, FcγRIIIa and FcγRIII), it is sufficient if the antibody, or the antigen-binding fragment thereof, binds with a higher affinity to one of human FcγRI, FcγRIIa and/or FcγRIII than to human FcγRIIb. More preferably, however, the antibody, or the antigen-binding fragment thereof, binds with a higher affinity to two of human FcγRI, FcγRIIa and/or FcγRIII than to human FcγRIIb. Most preferably, the antibody, or the antigen-binding fragment thereof, binds with a higher affinity to all three of human FcγRI, FcγRIIa and/or FcγRIII than to human FcγRIIb. In particular, (i) the antibody, or the antigen-binding fragment thereof, most preferably binds with a higher affinity to each of human FcγRI, FcγRIIa and/or FcγRIII than to human FcγRIIb—and/or (ii) the antibody, or the antigen-binding fragment thereof, most preferably binds with a higher affinity to each of those human FcγRI, FcγRIIa and/or FcγRIII, for which the antibody, or the antigen-binding fragment thereof, comprises a binding site, than to human FcγRIIb.

In general, human human FcγRI, FcγRIIa and FcγRIII are activating fc receptors, whereas human FcγRIIb is an inhibitory Fc receptor. Accordingly, an antibody, or antigen-binding fragment thereof, which binds with a higher affinity to human FcγRI, FcγRIIa and/or FcγRIII than to human FcγRIIb, as described above, binds rather to an activating Fc receptor than to an inhibitory Fc receptor, thereby shifting the activating/inhibiting ratio to the activating side and decreasing the regulatory influence of the inhibitory FcγRIIb. Accordingly, phagocytosis and direct killing are increased.

As used herein, a “higher affinity to human FcγRIIa than to human FcγRIIb” means in particular that the EC₅₀ (effective concentration at half maximum binding signals) of a certain antibody, or fragment thereof, for binding to FcγRIIa is lower than the EC₅₀ of the same antibody, or fragment thereof, for binding to FcγRIIIb. In other words, a lower concentration of the antibody (or of the fragment thereof) is required for half-maximum binding to FcγRIIa than for half-maximum binding to FcγRIIb. For example, the FcγRIIa/FcγRIIb ratio (which is >1 if the antibody binds with a higher affinity to human FcγRIIa than to human FcγRIIb), may be determined by EC₅₀(FcγRIIb)/EC₅₀(FcγRIIa). EC₅₀ values may be determined, for example, by standard enzyme-linked immunosorbent assay (ELISA). Alternatively, binding affinities of the antibody, or fragment thereof, may also be determined by surface plasmon resonance measurements, e.g. as described in Richards J O, Karki S, Lazar G A, Chen H, Dang W, Desjarlais J R (2008) Optimization of antibody binding to FcgammaRIIa enhances macrophage phagocytosis of tumor cells. Mol Cancer Ther 7:2517-2527). In general, the binding affinities of the antibody, or fragment thereof, to FcγRIIa and FcγRIIb can be determined by various methods known to the skilled person. However, the binding affinities of a certain antibody, or fragment thereof, to FcγRIIa and FcγRIIb are in particular obtained by using the same method to determine FcγRIIa and FcγRIIb binding affinities.

This applies in a similar manner for a “higher affinity to human FcγRI than to human FcγRIIb”, which means in particular that the EC₅₀ (effective concentration at half maximum binding signals) of a certain antibody, or fragment thereof, for binding to FcγRI is lower than the EC₅₀ of the same antibody, or fragment thereof, for binding to FcγRIIb. In other words, a lower concentration of the antibody (or of the fragment thereof) is required for half-maximum binding to FcγRI than for half-maximum binding to FcγRIIb. For example, the FcγRI/FcγRIIb ratio (which is >1 if the antibody binds with a higher affinity to human FcγRI than to human FcγRIIb), may be determined by EC₅₀(FcγRIIb)/EC₅₀(FcγRI). EC₅₀ values may be determined, for example, by standard enzyme-linked immunosorbent assay (ELISA). Alternatively, binding affinities of the antibody, or fragment thereof, may also be determined by surface plasmon resonance measurements, e.g. as described in Richards J O, Karki S, Lazar G A, Chen H, Dang W, Desjarlais J R (2008) Optimization of antibody binding to FcgammaRIIa enhances macrophage phagocytosis of tumor cells. Mol Cancer Ther 7:2517-2527). In general, the binding affinities of the antibody, or fragment thereof, to FcγRI and FcγRIIb can be determined by various methods known to the skilled person. However, the binding affinities of a certain antibody, or fragment thereof, to FcγRI and FcγRIIb are in particular obtained by using the same method to determine FcγRI and FcγRIIb binding affinities.

This applies also in a similar manner for a “higher affinity to human FcγRIII than to human FcγRIIb”, which means in particular that the EC₅₀ (effective concentration at half maximum binding signals) of a certain antibody, or fragment thereof, for binding to FcγRIII is lower than the EC₅₀ of the same antibody, or fragment thereof, for binding to FcγRIIb. In other words, a lower concentration of the antibody (or of the fragment thereof) is required for half-maximum binding to FcγRIII than for half-maximum binding to FcγRIIb. For example, the FcγRIII/FcγRIIb ratio (which is >1 if the antibody binds with a higher affinity to human FcγRIII than to human FcγRIIb), may be determined by EC₅₀/(FcγRIIb)/EC₅₀(FcγRII). EC₅₀ values may be determined, for example, by standard enzyme-linked immunosorbent assay (ELISA). Alternatively, binding affinities of the antibody, or fragment thereof, may also be determined by surface plasmon resonance measurements, e.g. as described in Richards J O, Karki S, Lazar G A, Chen H, Dang W, Desjarlais J R (2008) Optimization of antibody binding to FcgammaRIIa enhances macrophage phagocytosis of tumor cells. Mol Cancer Ther 7:2517-2527). In general, the binding affinities of the antibody, or fragment thereof, to FcγRIII and FcγRIIb can be determined by various methods known to the skilled person. However, the binding affinities of a certain antibody, or fragment thereof, to FcγRIII and FcγRIIb are in particular obtained by using the same method to determine FcγRIII and FcγRIIb binding affinities.

Preferably, antibody, or the antigen-binding fragment thereof, for use according to the present invention comprises a binding site for human FcγRI, wherein the antibody, or the antigen-binding fragment thereof, binds with a higher affinity to human FcγRI than to human FcγRIIb. It is also preferred that the antibody, or the antigen-binding fragment thereof, for use according to the present invention comprises a binding site for human FcγRIII, wherein the antibody, or the antigen-binding fragment thereof, binds with a higher affinity to human FcγRIII than to human FcγRIIb. Most preferably, the antibody, or the antigen-binding fragment thereof, for use according to the present invention comprises a binding site for human FcγRIIa, wherein the antibody, or the antigen-binding fragment thereof, binds with a higher affinity to human FcγRIIa than to human FcγRIIb.

In particular, an improved FcγRIIa/FcγRIIb binding ratio strongly increases antibody-dependent cellular phagocytosis (ADCP; Richards J O, Karki 5, Lazar G A, Chen H, Dang W, Desjarlais J R (2008) Optimization of antibody binding to FcgammaRIIa enhances macrophage phagocytosis of tumor cells. Mol Cancer Ther 7:2517-2527) and favors activation and maturation of DCs with a positive effect on the induction of tumor immunity (Boruchov A M, Heller G, Veri M C, Bonvini E, Ravetch J V, Young J W (2005) Activating and inhibitory IgG Fc receptors on human DCs mediate opposing functions. J Clin Invest 115:2914-2923; Kalergis A M, Ravetch J V (2002) Inducing tumor immunity through the selective engagement of activating Fcgamma receptors on dendritic cells. J Exp Med 195:1653-1659). In particular, Richards et al., 2008, showed that antibodies binding with higher affinity to human FcγRIIa than to human FcγRIIb mediate enhanced phagocytosis of antibody-coated target cells by macrophages (Richards J O, Karki S, Lazar G A, Chen H, Dang W, Desjarlais J R (2008) Optimization of antibody binding to FcgammaRIIa enhances macrophage phagocytosis of tumor cells. Mol Cancer Ther 7:2517-2527). Accordingly, the antibody, or the antigen binding fragment thereof, for use according to the present invention, which binds with a higher affinity to human FcγRIIa than to human FcγRIIb, triggers enhanced macrophage phagocytosis of tumor cells. Preferably, the antibody, or the antigen binding fragment thereof, for use according to the present invention binds with a higher affinity to the R131 form of human FcγRIIa than to human FcγRIIb.

Exemplary antibodies having a higher affinity to human FcγRIIa than to human FcγRIIb are known in the art. For example, Richards et al., 2008, describes a variant of IgG1, which comprises a G236A substitution, leading to considerable increase in the FcγRIIa/FcγRIIb ratio (Richards J O, Karki S, Lazar G A, Chen H, Dang W, Desjarlais J R (2008) Optimization of antibody binding to FcgammaRIIa enhances macrophage phagocytosis of tumor cells. Mol Cancer Ther 7:2517-2527). Moreover, Lindhofer et al., 2011 describe Triomab® antibodies having a mouse IgG2a/rat IgG2b Fc region, which also show high FcγRIIIa/FcγRIIb ratios (Lindhofer H, Hess J, Ruf P. Trifunctional Triomab® antibodies for cancer therapy. In: Kontermann RE (ed.), Bispecific antibodies. Springer, Berlin, 2011, p. 289-312), and, thus, enhanced phagocytosis of antibody-coated target cells by macrophages and increased direct killing of tumor cells.

Although the mere FcγRI, FcγRIIa or FcγRIII binding site is sufficient, it is preferred that the antibody, or the antigen binding fragment thereof, for use according to the present invention comprises an Fc moiety, in particular an Fc region.

As used herein, the term “Fc moiety” refers to a sequence derived from the portion of an immunoglobulin heavy chain beginning in the hinge region just upstream of the papain cleavage site and ending at the C-terminus of the immunoglobulin heavy chain. In particular, the “Fc moiety” comprises a binding site for FcγRI, FcγRIIa and/or FcγRIII. Preferably, the “Fc moiety” comprises a binding site for FcγRIIa. However, it is also preferred that an Fc moiety may mediate a functionality different from binding to an Fc receptor, for example binding to a protein of the complement system. In this case, the FcγRI, FcγRIIa and/or FcγRIII binding site may be present in the antibody separate from the Fc moiety. Accordingly, an “Fc moiety” may be a complete Fc region or a part (e.g., a domain) thereof. Preferably, the “Fc moiety” mediates the full functionality of a complete Fc region, e.g. including Fc receptor binding and, optionally, binding to a protein from the complement system. Thus, the antibody as used according to the present invention preferably comprises a complete Fc region, whereby a complete Fc region comprises at least a hinge domain, a CH2 domain, and a CH3 domain.

The Fc moiety may also comprise one or more amino acid insertions, deletions, or substitutions relative to a naturally-occurring Fc region. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof. Preferably, the Fc moiety comprises a G236A substitution. Preferably, the Fc moiety/Fc region of the antibody, or of the fragment thereof, binds to Fc receptor-positive cells, which preferably at least express(es) FcγRI, FcγRIIa and/or FcγRIII, more preferably at least FcγRIIa.

Preferably, the binding site for human FcγRI, FcγRIIa and/or FcγRIII (in particular the Fc moiety) in the antibody (or fragment thereof) for use according to the present invention is mouse IgG2a/rat IgG2b. This means that those parts of the antibody (or fragment thereof), which contribute to the binding to human FcγRI, FcγRIIa and/or FcγRIII, are mouse IgG2a and/or rat IgG2b sequences. In particular, the antibody (or fragment thereof) comprises the binding site for human FcγRI, FcγRIIa and/or FcγRIII (in particular the Fc moiety) of mouse IgG2a and/or rat IgG2b. It is well-known to the skilled person that mouse IgG2a and rat IgG2b comprise a binding site for human FcγRI, FcγRIIa and/or FcγRIII (in particular the Fc moiety), since well-known antibodies, which are approved for use in humans, such as catumaxomab, comprise an Fc moiety of mouse IgG2a/rat IgG2b. Accordingly, a combination of both ((i) the binding site for human FcγRI, FcγRIIa and/or FcγRIII, in particular the Fc moiety, of mouse IgG2a and (ii) the binding site for human FcγRI, FcγRIIa and/or FcγRIII, in particular the Fc moiety, of rat IgG2b) is preferred, for example as provided by heterologous antibodies as described herein. Most preferably, the antibody, or the fragment thereof, comprises a mouse IgG2a/rat IgG2b Fc region. This means in particular that the antibody's Fc region consists of (i) a mouse IgG2a Fc region chain and (ii) a rat IgG2b Fc region chain. In particular, one (heavy) chain of the antibody comprises the Fc region of a mouse IgG2a heavy chain and the other (heavy) chain of the antibody comprises the Fc region of a rat IgG2b heavy chain, and both form together the Fc region of the antibody as described herein (referred to as “mouse IgG2a/rat IgG2b Fc region”). Such antibodies show selectively enhanced binding to the activating FcγRIIa, but not to its inhibitory counterpart FcγRIIb (Lindhofer H, Hess J, Ruf P. Trifunctional Triomab® antibodies for cancer therapy. In: Kontermann RE (ed.), Bispecific antibodies. Springer, Berlin, 2011, p. 289-312).

Antibody Format

The antibody, or the antigen binding fragment thereof, for use according to the present invention may be of any antibody format as long as it includes the at least two specificities as described above and the FcγRI, FcγRIIa and/or FcγRIII binding site as described above. In particular, multifunctional antibodies preferably encompass “whole” antibodies, such as whole IgG- or IgG-like molecules, while antigen binding fragments in the context of the present invention preferably refer to small recombinant formats, such as tandem single chain variable fragment molecules (taFvs), diabodies (Dbs), single chain diabodies (scDbs) and various other derivatives of these (as described by Byrne H. et al. (2013) Trends Biotech, 31 (11): 621-632 with FIG. 2 showing various bispecific antibody formats; Weidle U. H. et al. (2013) Cancer Genomics and Proteomics 10: 1-18, in particular FIG. 1; and Chan, A. C. and Carter, P. J. (2010) Nat Rev Immu 10: 301-316, in particular FIG. 3). Preferred examples include, but are not limited to, Triomabs and quadroma antibodies.

Thus, the antibody or scaffold structure, or the antigen binding fragment thereof, for use according to the present invention may be selected from the group comprising Triomabs; hybrid hybridoma (quadroma); Multispecific anticalin platform (Pieris); Diabodies; Single chain diabodies; Tandem single chain Fv fragments; TandAbs, Trispecific Abs (Affimed) (105-110 kDa); Darts (dual affinity retargeting; Macrogenics); multifunctional recombinant antibody derivates (110 kDa); Dock and lock platform; Knob into hole (KIH) platform; Humanized bispecific IgG antibody (REGN1979) (Regeneron); Mab² bispecific antibodies (F-Star); DVD-Ig=dual variable domain immunoglobulin (Abbvie); kappa-lambda bodies; TBTI tetravalent bispecific tandem Ig; and CrossMab.

The antibody, or the antigen binding fragment thereof, for use according to the present invention may be selected from bispecific IgG-like antibodies (BsigG) comprising CrossMab; DAF (two-in-one); DAF (four-in-one); DutaMab; DT-IgG; Knobs-in-holes common LC; Knobs-in-holes assembly; Charge pair; Fab-arm exchange; SEEDbody; Triomab; LUZ-Y; Fcab; κλ-body; and Orthogonal Fab. These bispecific antibody formats are shown and described for example in Spiess C., Zhai Q. and Carter P. J. (2015) Molecular Immunology 67: 95-106, in particular FIG. 1 and corresponding description. e.g. p. 95-101.

Preferably, the antibody, or the antigen binding fragment thereof, for use according to the present invention may be selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)—IgG; IgG(L)-V; V(L)-IgG; KIH IgG-scFab; 2scFv-IgG; IgG-2scFv; scFv4-Ig; scFv4-1g; Zybody; and DVI-IgG (four-in-one). These bispecific antibody formats are shown and described for example in Spiess C., Zhai Q. and Carter P. J. (2015) Molecular Immunology 67: 95-106, in particular FIG. 1 and corresponding description, e.g. p. 95-101.

Preferably, the antibody, or the antigen binding fragment thereof, for use according to the present invention may be selected from bispecific antibody fragments comprising sc-Diabody-CH3; Diabody-CH3; Minibody; TriBi minibody; scFv-CH3 KIH; scFv-KIH; Fab-scFv-Fc; Tetravalent HCAb; scDiabody-Fc; Diabody-Fc; Tandem scFv-Fc; and Intrabody. These bispecific antibody formats are shown and described for example in Spiess C., Zhai Q. and Carter P. J. (2015) Molecular Immunology 67: 95-106, in particular FIG. 1 and corresponding description, e.g. p. 95-101.

In particular, the antibody, or the antigen binding fragment thereof, for use according to the present invention may be selected from bispecific antibody conjugates comprising IgG-IgG and Cov-X-Body. These bispecific antibody formats are shown and described for example in Spiess C., Zhai Q. and Carter P. J. (2015) Molecular Immunology 67: 95-106, in particular FIG. 1 and corresponding description, e.g. p. 95-101.

Preferably the antibody, or the antigen binding fragment thereof, for use according to the present invention is a bispecific trifunctional antibody.

Preferably, the antibody, or the antigen binding fragment thereof, for use according to the present invention has an IgG-like format (based on IgG, also referred to as “IgG type”), whereby an antibody having an IgG-like format usually comprises two heavy chains and two light chains. In general, Immunoglobulin G (IgG) is known as a type of antibody. It is understood herein as a protein complex composed of four peptide chains-two identical heavy chains and two identical light chains arranged in a Y-shape typical of antibody monomers. Each IgG has typically two antigen binding sites, which may be different or identical. Representing about 75% of serum antibodies in humans, IgG is the most common type of antibody found in the circulation. Physiologically, IgG molecules are created and released by plasma B cells.

Examples of an antibody having an IgG-like format include a quadroma and various IgG-scFv formats (cf: Byrne H. et al. (2013) Trends Biotech, 31 (11): 621-632; FIG. 2A-F), whereby a quadroma is preferred, which is preferably generated by fusion of two different hybridomas. Within the IgG class, antibodies may preferably be based on the IgG1, IgG2, IgG3 or IgG4 subclass, whereby an antibody based on IgG1 (also referred to as “IgG1 type”) or IgG2 (also referred to as “IgG2 type”) is preferred. The multifunctional antibodies or antigen binding fragments for use according to the present invention may alternatively be based on any immunoglobulin class (e.g., IgA, IgG, IgM etc.) and subclass (e.g. IgA1, IgA2, IgG1, IgG2, IgG3, IgG4 etc.)

Preferred bispecific IgG-like antibody formats comprise for example hybrid hybridoma (quadroma), knobs-into-holes with common light chain, various IgG-scFv formats, various scFv-IgG formats, two-in-one IgG, dual V domain IgG, IgG-V, and V-IgG, which are shown for example in FIG. 3c of Chan, A. C. and Carter, P. J. (2010) Nat Rev Immu 10: 301-316 and described in said article. Further preferred bispecific IgG-like antibody formats include for example DAF, CrossMab, IgG-dsscFv, DVD, IgG-dsFV, IgG-scFab, scFab-dsscFv and Fv2-Fc, which are shown in FIG. 1A of Weidle U. H. et al. (2013) Cancer Genomics and Proteomics 10: 1-18 and described in said article. Further preferred bispecific IgG-like antibody formats include DAF (two-in-one); DAF (four-in-one); DutaMab; DT-IgG; Knobs-in-holes assembly; Charge pair; Fab-arm exchange; SEEDbody; Triumab; LUZ-Y; Fcab; κλ-body; Orthogonal Fab; DVD-IgG; IgG(H)-scFv; scFv-(H)IgG; IgGU-scFv; scFV-(L)IgG; IgG(L,H)-Fv; IgG(H)-V; V(H)—IgG; IgG(L)-V; V(L)-IgG; KIH IgG-scFab; 2scFv-IgG; IgG-2scFv; scFv4-Ig scFv4-Ig; Zybody; and DVI-IgG (four-in-one) as shown and described for example in Spiess C., Zhai Q. and Carter P. J. (2015) Molecular Immunology 67: 95-106, in particular FIG. 1 and corresponding description, e.g. p. 95-101.

In general, production methods for antibodies are known in the art. Monoclonal antibodies originating from mammals, for example human, rat, mouse, rabbit, goat or sheep, can be produced by conventional methods, for example as described in Köhler and Milstein (Nature 256 (1975), 495), in Harlow and Lane (Antibodies, A Laboratory Manual (1988), Cold Spring Harbor) or in Galfie (Meth. Enzymol. 73 (1981), 3) or in DE 195 31 346. In particular, the multifunctional antibodies, or the antigen binding fragments thereof, for use according to the present invention can be produced by three main methods: (i) chemical conjugation, which involves chemical cross-linking; (ii) fusion of two different hybridoma cell lines (for example as described in Milstein et al., Nature 305 (1983), 537); or (iii) genetic approaches involving recombinant DNA technology (for example as described in Kurucz et al., J. Immunol. 154 (1995), 4576; Holliger et al., Proc. Natl. Acad. Sc. USA 90 (1993), 6444).

Preferably, the antibodies can be obtained by fusion of two different hybridoma cell lines (for example as described in Milstein et al., Nature 305 (1983), 537). Thereby, different hybridoma cell lines, each producing antibodies with one of the desired specificities, are fused and—among cell clones (“quadroma”) producing a heterogeneous antibody population—such quadroma (or “hybrid-hybridoma”), which secrete the desired multifunctional antibodies, can be identified and isolated.

Alternative approaches included chemical conjugation of two different mAbs and/or smaller antibody fragments. Oxidative reassociation strategies to link two different antibodies or antibody fragments were found to be inefficient due to the presence of side reactions during reoxidation of the multiple native disulfide bonds. Current methods for chemical conjugation focus on the use of homo- or hetero-bifunctional crosslinking reagents.

Recombinant DNA technology has yielded the greatest range of multifunctional antibodies, through artificial manipulation of genes and represents the most diverse approach for antibody generation (cf. Byrne H. et al. (2013) Trends Biotech, 31 (11): 621-632). Accordingly, multifunctional antibodies, are in particular obtained by recombinant DNA techniques or by (hybrid) hybridoma technologies.

Preferably, the antibody, or the antigen-binding fragment thereof, for use according to the present invention is a heterologous antibody, or a heterologous antibody fragment. As used herein, the term “heterologous” means that the antibody, or the antigen-binding fragment thereof, comprises heavy chains of distinct immunoglobulin subclasses (e.g., IgG1, IgG2, IgG3, and IgG4 in humans; e.g., IgG1, IgG2a, IgG2b, and IgG3 in mice and rats) and/or of distinct origin (species).

Preferably, the antibody, or the antigen-binding fragment thereof, for use according to the present invention comprises a heavy chain, which is derived from rat and/or mouse. “Derived” from rat and/or mouse means in particular that the antibody's amino acid sequence of the CH3 part of the heavy chain, preferably the antibody's amino acid sequence of the Fc region of the heavy chain, shares at least 95%, preferably at least 97%, more preferably at least 98%, even more preferably at least 99%, and most preferably 100% sequence identity with a CH3 part, or Fc region, respectively, of a rat and/or mouse immunoglobulin heavy chain.

Accordingly, antibodies “derived” from rat and/or mouse also includes antibodies comprising heavy chains, which share at least 95%, preferably at least 97%, more preferably at least 98%, even more preferably at least 99%, and most preferably 100% sequence identity with a rat and/or mouse immunoglobulin heavy chain over their entire length. Moreover, it is also preferred that the antibody, or the antigen-binding fragment thereof, for use according to the present invention is a rat and/or mouse antibody or antigen-binding fragment. This means that all heavy and light chains comprised by the antibody or antigen-binding fragment share at least 95%, preferably at least 97%, more preferably at least 98%, even more preferably at least 99%, and most preferably 100% sequence identity with a rat and/or mouse immunoglobulin heavy chain or light chain, respectively.

However, since the antibody, or the antigen-binding fragment thereof, for use according to the present invention is preferably for use in human subjects, it is preferred that at least the three CDRs (complementary-determining regions) and/or the framework regions of the heavy chain's (and light chain's) variable region are of human origin or humanized, in order to ensure the specificity against a (human) T cell surface antigen and the specificity against a (human) cancer- and/or tumor-associated antigen. Accordingly, a preferred antibody, or antigen-binding fragment thereof, for use according to the present invention comprises a heavy chain having (i) a CH3 part, preferably an Fc region, which shares at least 95%, preferably at least 97%, more preferably at least 98%, even more preferably at least 99%, and most preferably 100% sequence identity with a CH3 part, or Fc region, respectively, of a rat and/or mouse immunoglobulin heavy chain; and (ii) at least the three CDRs (complementary-determining regions) and/or the framework regions of the heavy chain's variable region are of human origin or humanized. More preferably, both heavy chains of the antibody, or antigen-binding fragment thereof, for use according to the present invention have (i) a CH3 part, preferably an Fc region, which shares at least 95%, preferably at least 97%, more preferably at least 98%, even more preferably at least 99%, and most preferably 100%/0 sequence identity with a CH3 part, or Fc region, respectively, of a rat and/or mouse immunoglobulin heavy chain; and (ii) at least the three CDRs (complementary-determining regions) and/or the framework regions of the heavy chain's variable region are of human origin or humanized. In addition, also the at least the three CDRs (complementary-determining regions) and/or the framework regions of the light chain's variable region are preferably of human origin or humanized.

It is particularly preferred that the antibody is a rat/mouse antibody, or antigen binding fragment thereof. As used herein, the term “rat/mouse antibody” refers to an antibody comprising
(a) a (heavy) chain, which differs from a rat (heavy) chain only in that the three CDRs and/or the framework regions of the heavy chain's variable region are of human origin or humanized (i.e. all sequences other than the CDRs and/or framework regions are rat (heavy) chain sequences); and
(b) a (heavy) chain, which differs from a mouse (heavy) chain only in that the three CDRs and/or the framework regions of the heavy chain's variable region are of human origin or humanized (i.e. all sequences other than the CDRs and/or framework regions are mouse (heavy) chain sequences).

Most preferably, the antibody is a mouse IgG2a/rat IgG2b antibody, or antigen binding fragment thereof. As used herein, the term “mouse IgG2a/rat IgG2b antibody” refers to an antibody comprising
(a) a (heavy) chain, which differs from a rat IgG2b (heavy) chain only in that the three CDRs and/or the framework regions of the heavy chain's variable region are of human origin or humanized (i.e. all sequences other than the CDRs and/or framework regions are rat IgG2b (heavy) chain sequences); and
(b) a (heavy) chain, which differs from a mouse IgG2a (heavy) chain only in that the three CDRs and/or the framework regions of the heavy chain's variable region are of human origin or humanized (i.e. all sequences other than the CDRs and/or framework regions are mouse IgG2a (heavy) chain sequences).

Preferably, the antibody, or the antigen-binding fragment thereof, for use according to the present invention is selected from one or more of the following isotype combinations (wherein every isotype/combination means in particular that at least the CDRs and/or the framework regions, preferably the variable regions, are preferably of human origin or humanized—even if the isotype refers to rat/mouse only):
Rat-IgG2b/Mouse-IgG2a,
Rat-IgG2b/Mouse-IgG2b,
Rat-IgG2b/Mouse-IgG3,
Rat-IgG2b/Human-IgG1,
Rat-IgG2b/Human-IgG2,
Rat-IgG2b/Human-IgG3 [oriental allotype G3m (st)=binding to protein A],
Rat-IgG2b/Human-IgG4,
Rat-IgG2b/Rat-IgG2c,
Mouse-IgG2a/Human-IgG3 [caucasian allotypes G3m (b+g)=no binding to protein A, labelled with * in the following],
Mouse-IgG2a/Mouse-[VH-CH1,VL-CL]-Human-IgG1-[Hinge]-Human-IgG3*-[CH2-CH3],
Mouse-IgG2a/Rat-[VH-CH1,VL-CL]-Human-IgG1-[Hinge]-Human-IgG3*-[CH2-CH3],
Mouse-IgG2a/Human-[VH-CH1,VL-CL]-Human-IgG1-[Hinge]-Human-IgG3*-[CH2-CH3],
Mouse-[VH-CH1,VL-CL]-Human-IgG1/Rat-[VH-CH1,VL-CL]-Human-IgG1-[Hinge]-Human-IgG3*-[CH2-CH3],
Mouse-[VH-CH1,VL-CL]-Human-IgG4/Rat-[VH-CH1,VL-CL]-Human-IgG4-[Hinge]-Human-IgG4[N-terminal region of CH2]-Human-IgG3*[C-terminal region of CH2: >amino acid position 251]-Human-IgG3*-[CH3],
Rat-IgG2b/Mouse-[VH-CH1,VL-CL]-Human-IgG1-[Hinge-CH2-CH3],
Rat-IgG2b/Mouse-[VH-CH1,VL-CL]-Human-IgG2-[Hinge-CH2-CH3],
Rat-IgG2b/Mouse-[VH-CH1,VL-CL]-Human-IgG3-[Hinge-CH2-CH3, oriental allotype],
Rat-IgG2b/Mouse-[VH-CH1,VL-CL]-Human-IgG4-[Hinge-CH2-CH3],
Human-IgG1/Human-[VH-CH1,VL-CL]-Human-IgG1-[Hinge]-Human-IgG3*-[CH2-CH3],
Human-IgG1/Rat-[VH-CH1,VL-CL]-Human-IgG1-[Hinge]-Human-IgG4[N-terminal region of CH2]-Human-IgG3*[C-terminal region of CH2: >amino acid position 251]-Human-IgG3-[CH3],
Human-IgG1/Mouse-[VH-CH1,VL-CL]-Human-IgG1-[Hinge]-Human-IgG4[N-terminal region of CH2]-Human-IgG3*[C-terminal region of CH2: >amino acid position 251]-Human-IgG3*-[CH3],
Human-IgG1/Rat-[VH-CH1,VL-CL]-Human-IgG1-[Hinge]-Human-IgG2[N-terminal region of CH2]-Human-IgG3*[C-terminal region of CH2: >amino acid position 251]-Human-IgG3*-[CH3],
Human-IgG1/Mouse-[VH-CH1,VL-CL]-Human-IgG1-[Hinge]-Human-IgG2[N-terminal region of CH2]-Human-IgG3*[C-terminal region of CH2: >amino acid position 251]-Human-IgG3-[CH3],
Human-IgG1/Rat-[VH-CH1,VL-CL]-Human-IgG1-[Hinge]-Human-IgG3*-[CH2-CH3],
Human-IgG1/Mouse-[VH-CH1,VL-CL]-Human-IgG1-[Hinge]-Human-IgG3*-[CH2-CH3],
Human-IgG2/Human-[VH-CH1,VL-CL]-Human-IgG2-[Hinge]-Human-IgG3*-[CH2-CH3],
Human-IgG4/Human-[VH-CH1,VL-CL]-Human-IgG4-[Hinge]-Human-IgG3*-[CH2-CH3],
Human-IgG4/Human-[VH-CH1,VL-CL]-Human-IgG4-[Hinge]-Human-IgG4[N-terminal region of CH2]-Human-IgG3*[C-terminal region of CH2: >amino acid position 251]-Human-IgG3*-[CH3],
Mouse-IgG2b/Rat-[VH-CH1,VL-CL]-Human-IgG1-[Hinge]-Human-IgG3*-[CH2-CH3],
Mouse-IgG2b/Human-[VH-CH1,VL-CL]-Human-IgG1-[Hinge]-Human-IgG3*-[CH2-CH3],
Mouse-IgG2b/Mouse-[VH-CH1,VL-CL]-Human-IgG1-[Hinge]-Human-IgG3*-[CH2-CH3],
Mouse-[VH-CH1,VL-CL]-Human-IgG4/Rat-[VH-CH1,VL-CL]-Human-IgG4-[Hinge]-Human-IgG4-[CH2]-Human-IgG3*-[CH3],
Human-IgG1/Rat[VH-CH1,VL-CL]-Human-IgG1[Hinge]-Human-IgG4-[CH2]-Human-IgG3*-[CH3],
Human-IgG1/Mouse[VH-CH1,VL-CL]-Human-IgG1[Hinge]-Human-IgG4-[CH2]-Human-IgG3*[CH3], and
Human-IgG4/Human[VH-CH1,VL-CL]-Human-IgG4-[Hinge]-Human-IgG4-[CH2]-Human-IgG3*-[CH3].

Preferably, the antibody, or the antigen binding fragment thereof, for use according to the invention is an IgG type (also referred to as “IgG-like”) antibody comprising a binding site for human FcγRI, FcγRIIa and/or FcγRIII, in particular an Fc region. More preferably, the antibody, or the antigen binding fragment thereof, for use according to the invention is a trifunctional bispecific antibody, which is a heterologous rat/mouse antibody comprising a binding site for human FcγRI, FcγRIIa and/or FcγRIII, in particular an Fc region. Thereby, an antibody with a subclass combination of mouse IgG2a and rat IgG2b is preferred. A heterologous rat/mouse antibody comprising a binding site for human Fc-RI, FcγRIIa and/or FcγRIII, in particular an Fc region, with heavy chains composed of murine IgG2a and rat IgG2b subclasses, each preferably with their respective light chains, is particularly preferred.

For antibodies comprising a binding site for human FcγRI, FcγRIIa and/or FcγRIII, in particular an Fc region, with heavy chains composed of murine IgG2a and rat IgG2b subclasses, in particular for antibodies of the Triomab format, it was shown that such antibodies bind with a higher affinity to human FcγRIIa than to human FcγRIIb and show a considerably improved FcγRIIa/FcγRIIb-binding ratio (Lindhofer H, Hess J, Ruf P. Trifunctional Triomab® antibodies for cancer therapy. In: Kontermann RE (ed.), Bispecific antibodies. Springer, Berlin, 2011, p. 289-312). Thus, such antibodies lead to enhanced phagocytosis of antibody-coated tumor cells by macrophages and increased direct killing of tumor cells. Accordingly, such antibodies are particularly preferred.

In general, the multifunctional antibody for use according to the invention exhibits preferably one of the following isotype combinations in its Fc-region: rat-IgG2b/mouse-IgG2a, rat-IgG2b/mouse-IgG2b, rat-IgG2b/human-IgG1, or mouse-[VH-CH1,VL-CL]-human-IgG1/rat-[VH-CH1,VL-CL]-human-IgG1-[hinge]-human IgG3*-[CH2-CH3], wherein * caucasian allotypes G3m(bf g)=no binding to protein A.

Most preferably, the antibody, or the antigen binding fragment thereof, for use according to the present invention is of the Triomab format. Triomabs are trifunctional, bispecific IgG-like antibodies having a specificity against CD3 and a specificity against a cancer- and/or tumor-associated antigen. These chimeras consist of two half antibodies, each with one light and one heavy chain, that originate from parental mouse IgG2a and rat IgG2b isotypes. Accordingly, the Fc region of Triomabs is mouse IgG2a/rat IgG2b.

Preferably, the antibody, or the antigen binding fragment thereof, for use according to the invention is selected from the group consisting of catumaxomab (anti-CD3×anti-EpCAM), FBTA05/lymphomun (anti-CD3×anti-CD20), ertumaxomab (anti-CD3×anti-HER2/neu), and/or ektomun (anti-CD3×anti-GD2), preferably the antibody is catumaxomab and/or ektomun.

The most preferred example of trifunctional bispecific antibodies is catumaxomab (Removab®) (anti-EpCAM×anti-CD3). Removab® was approved for the treatment of malignant ascites in 2009 by the EMA (Linke et al. Catumaxomab—clinical development and future directions. (2010) mAbs 2:2). Further preferred examples of trifunctional bispecific antibodies include (i) FBTAOS (also called “lymphomun”), a trifunctional anti-CD3×anti-CD20 antibody, (ii) ertumaxomab, a trifunctional anti-CD3×anti-HER2 antibody, (iii) ektomun, a trifunctional anti-CD3×anti-GD2 antibody, and (iv) TRBs02, a trifunctional antibody specific for human melanoma (Ruf et al. (2004) Int J Cancer, 108: 725-732).

Immune Checkpoint Modulator

As used herein (i.e. throughout the present specification), the term “immune checkpoint modulator” (also referred to as “checkpoint modulator”) refers to a molecule or to a compound that modulates (e.g., totally or partially reduces, inhibits, interferes with, activates, stimulates, increases, reinforces or supports) the function of one or more checkpoint molecules. Thus, an immune checkpoint modulator may be an “immune checkpoint inhibitor” (also referred to as “checkpoint inhibitor” or “inhibitor”) or an “immune checkpoint activator” (also referred to as “checkpoint activator” or “activator”). An “immune checkpoint inhibitor” (also referred to as “checkpoint inhibitor” or “inhibitor”) totally or partially reduces, inhibits, interferes with, or negatively modulates the function of one or more checkpoint molecules. An “immune checkpoint activator” (also referred to as “checkpoint activator” or “activator”) totally or partially activates, stimulates, increases, reinforces, supports or positively modulates the function of one or more checkpoint molecules. Immune checkpoint modulators are typically able to modulate (i) self-tolerance and/or (ii) the amplitude and/or the duration of the immune response. Preferably, the immune checkpoint modulator used according to the present invention modulates the function of one or more human checkpoint molecules and is, thus, a “human checkpoint modulator”. Preferably, the immune checkpoint modulator is an activator or an inhibitor of one or more immune checkpoint point molecule(s) selected from CD27, CD28, CD40, CD122, CD137, OX40, GITR, ICOS, A2AR, B7-H3, B7-H4, BT LA (CD272), CD40, CTLA-4, IDO, KIR, LAG3, PD-1, PD-L1, PD-L2, TIM-3, VISTA, CEACAM1, GARP, PS, CSF1R, CD94/NKG2A, TDO, GITR, TNFR and/or FasR/DcR3; or an activator or an inhibitor of one or more ligands thereof.

Checkpoint molecules (also referred to as “immune checkpoint molecules” or “immune checkpoints”) are molecules, such as proteins, which are typically involved in immune pathways and, for example, regulate T-cell activation, T-cell proliferation and/or T-cell function. Accordingly, the function of checkpoint molecules, which is modulated (e.g., totally or partially reduced, inhibited, interfered with, activated, stimulated, increased, reinforced or supported) by checkpoint modulators, is typically the (regulation of) T-cell activation, T-cell proliferation and/or T cell function. Immune checkpoint molecules thus regulate and maintain self-tolerance and the duration and amplitude of physiological immune responses. Many of the immune checkpoint molecules belong to the B7:CD28 family or to the tumor necrosis factor receptor (TNFR) super family and, by binding to specific ligands, activate signaling molecules that are recruited to the cytoplasmic domain (cf. Susumu Suzuki et al., 2016: Current status of immunotherapy. Japanese Journal of Clinical Oncology, 2016: doi: 10.1093/jjco/hyv201 [Epub ahead of print]; in particular Table 1).

The B7:CD28 family comprises the most frequently targeted pathways in immune checkpoint research including the CTLA-4—B7-1/17-2 pathway and the PD-1—B7-H1(PDL1)/87-DC(PD-L2) pathway. Another member of this family is ICOS-ICOSL/B7-H2. Further members of that family include CD28, B7-H3 and B7-H4.

CD28 is constitutively expressed on almost all human CD4+ T cells and on around half of all CD8 T cells. Binding with its two ligands are CD80 (17-1) and CD86 (B7-2), expressed on dendritic cells, prompts T cell expansion. The co-stimulatory checkpoint molecule CD28 competes with the inhibitory checkpoint molecule CTLA4 for the same ligands, CD80 and CD86 (cf. Buchbinder E. I. and Desai A., 2016: CTLA-4 and PD-1 Pathways—Similarities, Differences and Implications of Their Inhibition; American Journal of Clinical Oncology, 39(1): 98-106).

Cytotoxic T-Lymphocyte-Associated protein 4 (CTLA4; also known as CD152) is a CD28 homolog with much higher binding affinity for B7. The ligands of CTLA-4 are CD80 (B7-1) and CD86 (87-2), similarly to CD28. However, unlike CD28, binding of CTLA4 to B7 does not produce a stimulatory signal, but prevents the co-stimulatory signal normally provided by CD28. Moreover, CTLA4 binding to B7 is assumed to even produce an inhibitory signal counteracting the stimulatory signals of CD28:B7 and TCR:MHC binding. CTLA-4 is considered the “leader” of the inhibitory immune checkpoints, as it stops potentially autoreactive T cells at the initial stage of naïve T-cell activation, typically in lymph nodes (Buchbinder E. I. and Desai A., 2016: CTLA-4 and PD-1 Pathways: Similarities, Differences and Implications of Their Inhibition; American Journal of Clinical Oncology, 39(1): 98-106). Preferred checkpoint inhibitors of CTLA4 include the monoclonal antibodies Yervoy® (Ipilimumab; Bristol Myers Squibb) and Tremelimumab (Pfizer/MedImmune). Further preferred CTLA-4 inhibitors include the anti-CTLA4 antibodies disclosed in WO 2001/014424, in WO 2004/035607, in US 2005/0201994, and in EP 1212422 B1. Additional preferred CTLA-4 antibodies are described in U.S. Pat. No. 5,811,097, in U.S. Pat. No. 5,855,887, in U.S. Pat. No. 6,051,227, in U.S. Pat. No. 6,984,720, in WO 01/14424 in WO 00/37504, in US 2002/0039581 and in US 2002/086014. Other preferred anti-CTLA-4 antibodies that can be used in the context of the present invention include, for example, those disclosed in WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), in U.S. Pat. Nos. 5,977,318, 6,682,736, 7,109,003, and in U.S. Pat. No. 7,132,281. In the context of the present invention, CTLA-4 is a particularly preferred checkpoint molecule.

Programmed Death 1 receptor (PD1) has two ligands, PD-L1 (also known as B7-H1 and CD274) and PD-L2 (also known as 87-DC and CD273). The PD1 pathway regulates previously activated T cells at the later stages of an immune response, primarily in peripheral tissues. An advantage of targeting PD1 is thus that it can restore immune function in the tumor microenvironment. Preferred inhibitors of the PD1 pathway include Opdivo® (Nivolumab; Bristol Myers Squibb), Keytruda® (Pembrolizumab; Merck), Durvalumab (Medimmune/AstraZeneca), MEDI4736 (AstraZeneca; cf. WO 2011/066389 A1), Atezolizumab (MPDL3280A, Roche/Genentech; cf. U.S. Pat. No. 8,217,149 B2), Pidilizumab (CT-011; CureTech), MEDI0680 (AMP-514; AstraZeneca), Avelumab (Merck), MSB-0010718C (Merck), PDR001 (Novartis), BMS-936559 (Bristol Myers Squibb), REGN2810 (Regeneron Pharmaceuticals), MIH1 (Affymetrix), AMP-224 (Amplimmune, GSK), BGB-A317 (BeiGene) and Lambrolizumab (e.g. disclosed as hPD109A and its humanized derivatives h409A11, h409A16 and h409A17 in WO2008/156712; Hamid et al., 2013; N. Engl. J. Med. 369:134-144).

Inducible T-cell costimulator (ICOS; also known as CD278) is expressed on activated I cells. Its ligand is ICOSL (B7-H2; CD275), expressed mainly on B cells and dendritic cells. The molecule seems to be important in T cell effector function.

B7-H3 (also known as CD276) was originally understood to be a co-stimulatory molecule but is now regarded as co-inhibitory. A preferred checkpoint inhibitor of B7-H3 is the Fc-optimized monoclonal antibody Enoblituzumab (MGA271; MacroGenics; cf. US 2012/0294796 A1).

B7-H4 (also known as VTCN1), is expressed by tumor cells and tumor-associated macrophages and plays a role in tumor escape. Preferred B7-H4 inhibitors are the antibodies described in Dangaj, D. et al., 2013; Cancer Research 73(15): 4820-9 and in Table 1 and the respective description of Jenessa B. Smith et al., 2014: B7-H4 as a potential target for immunotherapy for gynecologic cancers: A closer look. Gynecol Oncol 134(1): 181-189. Other preferred examples of B7-H4 inhibitors include antibodies to human 67-H4 as disclosed, e.g., in WO 2013/025779 A1 and in WO 2013/067492 A1 or soluble recombinant forms of B7-H4, such as disclosed in US 2012/0177645 A1.

The TNF superfamily comprises in particular 19 protein-ligands binding to 29 cytokine receptors. They are involved in many physiological responses such as apoptosis, inflammation or cell survival (Croft, M., C. A. Benedict, and C. F. Ware, Clinical targeting of the TNF and TNFR superfamilies. Nat Rev Drug Discov, 2013.12(2): p. 147-68). The following checkpoint molecules/pathways are preferred for cancer indications: TNFRSF4 (OX40/OX40L), TNFRSFS (CD40UCD40), TNFRSF7 (CD27/CD70), TNFRSF8 (CD30/CD30L), TNFRSF9 (4-11BB/4-1BBL), TNFRSF10 (TRAILR/TRAIL)), TNFRSF12 (FN14/TWEAK), TNFRSF13 (BAFFRTACI/APRIL-BAFF) and TNFRSF18 (GITR/GITRL). Further preferred checkpoint molecules/pathways include Fas-Ligand and TNFRSF1 (TNFα/TNFR). Moreover, the B- and T-lymphocyte attenuator (BTLA)/herpes virus entry mediator (HVEM) pathway are preferred for enhancing immune responses, just like the CTLA-4 blockade. Accordingly, in the context of the present invention such checkpoint modulators are preferred for the use in the treatment and/or prevention in cancer, which modulate one or more checkpoint molecules selected from TNFRSF4 (OX40/OX40L), TNFRSFS (CD40L/CD40), TNFRSF7 (CD27/CD70), TNFRSF9 (4-1BB/4-1BBL), TNFRSF18 (GITR/GITRL), FasR/DcR3/Fas ligand, TNFRSF1 (TNFα/TNFR), BTLA/HVFM and CTLA4.

OX40 (also known as CD134 or TNFRSF4) promotes the expansion of effector and memory T cells, but it is also able to suppress the differentiation and activity of T-regulatory cells and to regulate cytokine production. The ligand of OX40 is OX40L (also known as TNFSF4 or CD252). OX40 is transiently expressed after T-cell receptor engagement and is only upregulated on the most recently antigen-activated T cells within inflammatory lesions. Preferred checkpoint modulators of OX40 include MEDI6469 (MedImmune/AstraZeneca), MEDI6383 (Medimmune/AstraZeneca), MEDI0562 (MedImmune/AstraZeneca), MOXR0916 (RG7888; Roche/Genentech) and GSK3174998 (GSK).

CD40 (also known as TNFRSF5) is expressed by a variety of immune system cells including antigen presenting cells. Its ligand is CD40L, also known as CD154 or TNFSF5, is transiently expressed on the surface of activated CD4+ T cells. CD40 signaling “licenses” dendritic cells to mature and thereby trigger T-cell activation and differentiation. However, CD40 can also be expressed by tumor cells. Thus, stimulation/activation of CD40 in cancer patients can be beneficial or deleterious. Accordingly, stimulatory and inhibitory modulators of this immune checkpoint were developed (Sufia Butt Hassan, Jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104). Preferred examples of CD40 checkpoint modulators include (i) agonistic anti-CD antibodies as described in Sufia Butt Hassan, Jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104, such as Dacetuzumab (SGN-40), CP-870893, FGK 4.5/FGK 45 and FGK115, preferably Dacetuzumab, and (ii) antagonistic anti-CD antibodies as described in Sufia Butt Hassan, jesper Freddie Sorensen, Barbara Nicola Olsen and Anders Elm Pedersen, 2014: Anti-CD40-mediated cancer immunotherapy: an update of recent and ongoing clinical trials, Immunopharmacology and Immunotoxicology, 36:2, 96-104, such as Lucatumumab (HCD122, CHIR-12.12). Further preferred immune checkpoint modulators of CD40 include SEA-CD40 (Seattle Genetics), ADC-1013 (Alligator Biosciences), APX005M (Apexigen Inc) and R07009789 (Roche).

CD27 (also known as TNFRSF7) supports antigen-specific expansion of naïve T cells and plays an important role in the generation of T cell memory. CD27 is also a memory marker of B cells. The transient availability of its ligand, CD70 (also known as TNFSF7 or CD27L), on lymphocytes and dendritic cells regulates the activity of CD27. Moreover, CD27 co-stimulation is known to suppress Th17 effector cell function. A preferred immune checkpoint modulator of CD27 is Varlilumab (Celldex). Preferred immune checkpoint modulators of CD70 include ARGX-110 (arGEN-X) and SGN-CD70A (Seattle Genetics).

CD137 (also known as 4-1BB or TNFRSF9) is a member of the tumor necrosis factor (TNF) receptor family and is increasingly associated with costimulatory activity for activated T cells. In particular, CD137 signaling (via its ligand CD137L, also known as TNFSF9 or 4-1BBL) results in T-cell proliferation and protects T cells, in particular, CD81 T cells, from activation-induced cell death. Preferred checkpoint modulators of CD137 include PF-05082566 (Pfizer) and Urelumab (BMS).

Glucocorticoid-Induced TNFR family Related gene (GITR, also known as TNFRSF18), prompts T cell expansion, including Treg expansion. The ligand for GITR (GITRL, TNFSF18) is mainly expressed on antigen presenting cells. Antibodies to GITR have been shown to promote an anti-tumor response through loss of Treg lineage stability. Preferred checkpoint modulators of GITR include BMS-986156 (Bristol Myers Squibb), TRX518 (GITR Inc) and MK-4166 (Merck).

Another preferred checkpoint molecule to be modulated is BTLA. B and T Lymphocyte Attenuator (BTLA; also known as CD272) is in particular expressed by CD8+ T cells, wherein surface expression of BTLA is gradually downregulated during differentiation of human CD8+T cells from the naïve to effector cell phenotype. However, tumor-specific human CD8+ T cells express high levels of BTLA. BTLA expression is induced during activation of T cells, and BTLA remains expressed on Th1 cells but not Th2 cells. Like PD1 and CTLA4, BTLA interacts with a 87 homolog, B7H4. However, unlike PD-1 and CTLA-4, BTLA displays T-Cell inhibition via interaction with tumor necrosis family receptors (TNF-R), not just the 87 family of cell surface receptors. BTLA is a ligand for tumor necrosis factor (receptor) superfamily, member 14 (TNFRSF14), also known as herpes virus entry mediator (HVEM; Herpesvirus Entry Mediator, also known as CD270). BTLA-HVEM complexes negatively regulate T-cell immune responses. Preferred BTLA inhibitors are the antibodies described in Table 1 of Alison Crawford and E. John Wherry, 2009: Editorial: Therapeutic potential of targeting BTLA. Journal of Leukocyte Biology 86: 5-8, in particular the human antibodies thereof. Other preferred antibodies in this context, which block human BTLA interaction with its ligand are disclosed in WO 2011/014438, such as “4C7” as described in WO 2011/014438.

Another checkpoint molecule family includes checkpoint molecules related to the two primary class of major histocompatibility complex (MHC) molecules (MHC class I and class II). This family includes killer Ig-like Receptor (KIR) for class I and lymphocyte activation gene-3 (LAG-3) for class II.

Killer-cell immunoglobulin-like Receptor (KIR) is a receptor for MHC Class I molecules on Natural Killer cells. An exemplary inhibitor of KIR is the monoclonal antibody Lirilumab (IPH 2102; Innate Pharma/BMS; cf. U.S. Pat. No. 8,119,775 B2 and Benson et al., 2012, Blood 120:4324-4333).

Lymphocyte Activation Gene-3 (LAG3, also known as CD223) signaling leads to suppression of an immune response by action to Tregs as well as direct effects on CD8+ T cells. A preferred example of a LAG3 inhibitor is the anti-LAG3 monoclonal antibody BMS-986016 (Bristol-Myers Squibb). Other preferred examples of a LAG3 inhibitor include LAG525 (Novartis), IMP321 (Immutep) and LAG3-Ig as disclosed in WO 2009/044273 A2 and in Brignon et al., 2009, Clin. Cancer Res. 15: 6225-6231 as well as mouse or humanized antibodies blocking human LAG3 (e.g., IMP701 as described in WO 2008/132601 A1), or fully human antibodies blocking human LAG3 (such as disclosed in EP 2320940 A2).

Another checkpoint molecule pathway is the TIM-3/GAL9 pathway.). T-cell Immunoglobulin domain and Mucin domain 3 (TIM-3, also known as HAVcr-2) is expressed on activated human CD4+ T cells and regulates Th1 and Th17 cytokines. TIM-3 acts as a negative regulator of Th1/Tc1 function by triggering cell death upon interaction with its ligand, galectin-9 (GAL9). TIM-3 is a T helper type 1 specific cell surface molecule that is regulating the induction of peripheral tolerance. A recent study has indeed demonstrated that TIM-3 antibodies could significantly enhance antitumor immunity (Ngiow, S. F., et al., Anti-TIM3 antibody promotes T cell IFN-gamma mediated antitumor immunity and suppresses established tumors. Cancer Res, 2011. 71(10): p. 3540-51). Preferred examples of TIM-3 inhibitors include antibodies targeting human TIM3 (e.g. as disclosed in WO 2013/006490 A2) or, in particular, the anti-human TIM3 blocking antibody F38-2E2 as disclosed by Jones et al., 2008, J Exp Med. 205 (12): 2763-79.

CEACAM1 (Carcinoembryonic antigen-related cell adhesion molecule 1) is a further checkpoint molecule (Huang, Y. H., et al., CFACAM1 regulates TIM-3-mediated tolerance and exhaustion. Nature, 2015. 517(7534): p. 386-90; Gray-Owen, S. D. and R. S. Blumberg, CFACAM1: contact-dependent control of immunity. Nat Rev Immunol, 2006. 6(6): p. 433-46). A preferred checkpoint modulator of CEACAM1 is CM-24 (cCAM Biotherapeutics).

Another immune checkpoint molecule is GARP, which plays a role in the ability of tumors to escape the patient's immune system. Presently in clinical trials, the candidate (ARGX-115) seems demonstrating interesting effect. Accordingly, ARGX-115 is a preferred GARP checkpoint modulator.

Moreover, various research groups have demonstrated that another checkpoint molecule is phosphatidylserine (also referred to as “PS”) may be targeted for cancer treatment (Creelan, B. C., Update on immune checkpoint inhibitors in lung cancer. Cancer Control, 2014. 2111): p. 80-9; Yin, Y., et al., Phosphatidylserine-targeting antibody induces Ml macrophage polarization and promotes myeloid-derived suppressor cell differentiation. Cancer Immunol Res, 2013. 1(4): p. 256-68). A preferred checkpoint modulator of phosphatidylserine (PS) is Bavituximab (Peregrine).

Another checkpoint pathway is CSF1/CSF1R (Zhu, Y., et al., CSF1/CSF1R Blockade Reprograms Tumor-Infiltrating Macrophages and Improves Response to T-cell Checkpoint Immunotherapy in Pancreatic Cancer Models. Cancer Research, 2014. 74(18): p. 5057-5069). Preferred checkpoint modulators of CSF1R include FPA008 (FivePrime), IMC-CS4 (Eli-Lilly), PLX3397 (Plexxicon) and R05509554 (Roche).

Furthermore, the CD94/NKG2A natural killer cell receptor is evaluated for its role in cervical carcinoma (Sheu, B. C., et al., Up-regulation of inhibitory natural killer receptors CD94/NKG2A with suppressed intracellular perforin expression of tumor infiltrating CD8+ T lymphocytes in human cervical carcinoma. Cancer Res, 2005. 65(7): p. 2921-9) and in leukemia (Tanaka, J., et al., Cytolytic activity against primary leukemic cells by inhibitory NK cell receptor (CD94/NKG2A)-expressing T cells expanded from various sources of blood mononuclear cells. Leukemia, 2005. 19(3): p. 486-9). A preferred checkpoint modulator of NKG2A is IPH2201 (innate Pharma).

Another preferred checkpoint molecule is IDO, the indoleamine 2,3-dioxygenase enzyme from the kynurenine pathway (Ball, H. J., et al., Indoleamine 2,3-dioxygenase-2; a new enzyme in the kynurenine pathway. Int j Biochem Cell Biol, 2009. 41(3): p. 467-71). Indoleamine 2,3-dioxygenase (IDO) is a tryptophan catabolic enzyme with immune-inhibitory properties. IDO is known to suppress T and NK cells, generate and activate Tregs and myeloid-derived suppressor cells, and promote tumour angiogenesis. IDO1 is overexpressed in many cancer and was shown to allow tumor cells escaping from the immune system (Liu, X., et al., Selective inhibition of IDO1 effectively regulates mediators of antitumor immunity. Blood, 2010. 115(17): p. 3520-30; Ino, K., et al., Inverse correlation between tumoral indoleamine 2,3-dioxygenase expression and tumor-infiltrating lymphocytes in endometrial cancer its association with disease progression and survival. Clin Cancer Res, 2008. 14(8): p. 2310-7) and to facilitate chronic tumor progression when induced by local inflammation (Muller, A. J., et al., Chronic inflammation that facilitates tumor progression creates local immune suppression by inducing indoleamine 2,3 dioxygenase. Proc Natl Acad Sci USA, 2008. 105(44): p. 17073-8). Preferred IDO inhibitors include Exiguamine A, epacadostat (INCB024360; InCyte), Indoximod (NewLink Genetics), NLG919 (NewLink Genetics/Genentech), GDC-0919 (NewLink Genetics/Genentech), F001287 (Flexus Biosciences/BMS) and small molecules such as 1-methyl-tryptophan, in particular 1-methyl-IDI-tryptophan and the IDO inhibitors listed in Table 1 of Sheridan C, 2015: IDO inhibitors move center stage in immune-oncology; Nature Biotechnology 33: 321.322.

Another preferred immune checkpoint molecule to be modulated is also a member of the kynurenine metabolic pathway: TDO (tryptophan-2,3-dioxygenase). Several studies already demonstrated the interest of TDO in cancer immunity and autoimmunity (Garber, K., Evading immunity: new enzyme implicated in cancer. J Natl Cancer Inst. 2012. 104(5): p. 349-52; Platten, M., W. Wick, and B. J. Van den Eynde, Tryptophan catabolism in cancer: beyond IDO and tryptophan depletion. Cancer Res, 2012. 72(21): p. 5433-40; Platten, M., et al., Cancer Immunotherapy by Targeting IDO1/TDO and Their Downstream Effectors. Front Immunol, 2014. 5: p. 673).

Another preferred immune checkpoint molecule to be modulated is A2AR. The Adenosine A2A receptor (A2AR) is regarded as an important checkpoint in cancer therapy because the tumor microenvironment has typically relatively high concentrations of adenosine, which is activating A2AR. Such signaling provides a negative immune feedback loop in the immune microenvironment (for review see Robert D. Leone et al., 2015: A2aR antagonists: Next generation checkpoint blockade for cancer immunotherapy. Computational and Structural Biotechnology journal 13: 265-272). Preferred A2AR inhibitors include Istradefylline, PBS-509, ST1535, ST4206, Tozadenant, V81444, Preladenant, Vipadenant, SCH58261, SYN115, ZM241365 and FSPTP.

Another preferred immune checkpoint molecule to be modulated is VISTA. V-domain Ig suppressor of r cell activation (VISTA; also known as C10orf54) is primarily expressed on hematopoietic cells so that consistent expression of VISTA on leukocytes within tumors may allow VISTA blockade to be effective across a broad range of solid tumors. A preferred VISTA inhibitor is JNJ-61610588 (ImmuNext), an anti-VISTA antibody, which recently entered a phase 1 clinical trial.

Another immune checkpoint molecule is CD122. CD122 is the Interleukin-2 receptor beta sub-unit. CD122 increases proliferation of CD8+ effector T cells.

The most preferred examples of checkpoint molecules include the “CTLA4-pathway” and the “PD1-pathway” with CTLA4 and its ligands CD80 and CD86 as well as PD1 with its ligands PD-L1 and PD-L2 (more details on CTLA4 and PD-1 pathways as well as further participants are described in Buchbinder E. I. and Desai A., 2016: CTLA-4 and PD-1 Pathways—Similarities, Differences and Implications of Their Inhibition; American Journal of Clinical Oncology, 39(1): 98-106). In more general, preferred examples of checkpoint molecules include CD27, CD28, CD40, CD122, CD137, OX40, GITR, ICOS, A2AR, B7-H3, 67-H4, BTLA, CD40, CTLA-4, IDO, KIR, LAG3, PD-1, TIM-3, VISTA, CEACAM1, GARP, PS, CSF1R, CD94/NKG2A, TDO, GITR, TNFR and/or FasR/DcR3 as well as, in particular, their ligands.

However, it may also be preferred that the immune checkpoint modulator is not an anti-CD28 antibody. More preferably the immune checkpoint modulator is not directed to CD28 (i.e., CD28 is preferably not a target of the immune checkpoint modulator as defined herein).

Moreover, it may also be preferred that the immune checkpoint modulator is not an inhibitor of PD-1. More preferably, the immune checkpoint modulator is not an inhibitor/antagonist of the PD-1 pathway (also referred to as “PD-1 axis”, which includes, in addition to PD-1 itself, also its ligands PD-L1 and PD-L2).

Immune checkpoint molecules are responsible for co-stimulatory or inhibitory interactions of T-cell responses. Accordingly, checkpoint molecules can be divided into (i) (co-)stimulatory checkpoint molecules and (ii) inhibitory checkpoint molecules. Typically, (co-)stimulatory checkpoint molecules act positively in concert with T-cell receptor (TCR) signaling induced by antigen stimulation, whereas inhibitory checkpoint molecules negatively regulate TCR signaling. Examples of (co-)stimulatory checkpoint molecules include CD27, CD28, CD40, CD122, CD137, OX40, GITR and ICOS. Examples of inhibitory checkpoint molecules include CTLA4 as well as PD1 with its ligands PD-L1 and PD-L2; and A2AR, B7-113, B7-114, BTLA, IDO, KIR, LAG3, TIM-3, VISTA, CEACAM1, GARP, PS, CSF1R, CD94/NKG2A, TDO, TNFR and fasR/DcR3.

Preferably, the immune checkpoint modulator is an activator of a (co-)stimulatory checkpoint molecule or an inhibitor of an inhibitory checkpoint molecule or a combination thereof.

Accordingly, the immune checkpoint modulator is more preferably (i) an activator of CD27, CD28, CD40, CD122, CD137, OX40, GITR and/or ICOS or (ii) an inhibitor of A2AR, B7-H3, B7-H4, BTLA, CD40, CTLA-4, IDO, KIR, LAG3, PD-1, PDL-1, PD-L2, TIM-3, VISTA, CFACAM1, GARP, PS, CSF1R, CD94/NKG2A, TDO, TNFR and/or FasR/DcR3.

As described above, a number of CD27, CD28, CD40, CD122, CD137, OX40, GITR, ICOS, A2AR, B7-H3, B7-H4, CTLA-4, PD1, PDL-1, PD-L2, IDO, LAG-3, BTLA, TIM3, VISTA, KIR, CEACAM1, GARP, PS, CSF1R, CD94/NKG2A, TDO, TNFR and/or FasR/DcR3 modulators (inhibitors/activators) are known and some of them are already in clinical trials or even approved. Based on these known immune checkpoint modulators, alternative immune checkpoint modulators may be developed in the (near) future. In particular, known modulators of the preferred immune checkpoint molecules may be used as such or analogues thereof may be used, in particular chimerized, humanized or human forms of antibodies.

More preferably, the immune checkpoint modulator is an inhibitor of an inhibitory checkpoint molecule (but preferably no inhibitor of a stimulatory checkpoint molecule).

Accordingly, the immune checkpoint modulator is even more preferably an inhibitor of A2AR, B7-H3, B7-H4, BTLA, CTLA-4, IDO, KIR, LAG3, PD-1, TIM-3, VISTA, CEACAM1, GARP, PS, CSF1R, CD94/NKG2A, TDO, TNFR and/or DcR3 or of a ligand thereof.

It is also preferred that the immune checkpoint modulator is an activator of a stimulatory or costimulatory checkpoint molecule (but preferably no activator of an inhibitory checkpoint molecule). Accordingly, the immune checkpoint modulator is more preferably an activator of CD27, CD28, CD40, CD122, CD137, OX40, GITR and/or ICOS or of a ligand thereof.

It is more preferred that the immune checkpoint modulator is an inhibitor of CTLA-4, PD-1, PD-L1 and/or PD-L2, even more preferably the immune checkpoint modulator is an inhibitor of CTLA-4, PD-1 and/or PD-L1, and most preferably the immune checkpoint modulator is an inhibitor of CTLA-4 and/or PD-1. An inhibitor of CTLA-4 is particularly preferred.

Accordingly, the checkpoint modulator may be selected from known inhibitors of the CTLA-4 pathway and/or the PD-1 pathway. Preferred inhibitors of the CTLA-4 pathway and of the PD-1 pathway include the monoclonal antibodies Yervoy® (Ipilimumab; Bristol Myers Squibb) and Tremelimumab (Pfizer/Medimmune) as well as Opdivo® (Nivolumab; Bristol Myers Squibb), Keytruda® (Pembrolizumab; Merck), Durvalumab (MedImmune/AstraZeneca), MEDI4736 (AstraZeneca; cf. WO 2011/066389 A1), MPDL3280A (Roche/Genentech; cf. U.S. Pat. No. 8,217,149 B2), Pidilizumab (CT-011; CureTech), MEDI0680 (AMP-514; AstraZeneca), MSB-0010718C (Merck), MIH1 (Affymetrix) and Lambrolizumab (e.g. disclosed as hPD109A and its humanized derivatives h409A11, h409A16 and h409A17 in WO2008/156712; Hamid et al., 2013; N. Engl. J. Med. 369:134-144). More preferred checkpoint inhibitors include the CTLA-4 inhibitors Yervoy® (Ipilimumab; Bristol Myers Squibb) and Tremelimumab (Pfizer/MedImmune) and/or the PD-1 inhibitors Opdivo® (Nivolumab; Bristol Myers Squibb), Keytruda® (Pembrolizumab; Merck), Pidilizumab (CT-011; CureTech), MEDI0680 (AMP-514; AstraZeneca), AMP-224 and Lambrolizumab (e.g. disclosed as hPD109A and its humanized derivatives h409A11, h409A16 and h409A17 in WO2008/156712; Hamid O. et al., 2013; N. Engl. J. Med. 369:134-144).

In the context of the present invention it is preferred if more than one immune checkpoint modulator (e.g., checkpoint inhibitor) is used, in particular at least 2, 3, 4, 5, 6, 7, 8, 9 or 10 distinct immune checkpoint modulators (e.g., checkpoint inhibitors) are used, preferably 2, 3, 4 or 5 distinct immune checkpoint modulators (e.g., checkpoint inhibitors) are used, more preferably 2, 3 or 4 distinct immune checkpoint modulators (e.g., checkpoint inhibitors) are used, even more preferably 2 or 3 distinct immune checkpoint modulators (e.g., checkpoint inhibitors) are used and most preferably 2 distinct immune checkpoint modulators (e.g., checkpoint inhibitors) are used. Thereby, “distinct” immune checkpoint modulators (e.g., checkpoint inhibitors) means in particular that they modulate (e.g., inhibit) different checkpoint molecule pathways.

Preferably, an inhibitor of the PD-1 pathway is combined with an inhibitor of the CTLA-4 pathway. For example, as described above a combination therapy with Nivolumab (anti-PD1) and Ipilimumab (anti-CTLA4) was approved by the FDA in 2015 for the treatment of patients with BRAF V600 wild-type, unresectable or metastatic melanoma. In addition, a successful phase 1b study on the combination of Durvalumab (anti-PD-L1) and Tremelimumab (anti-CTLA4) in non-small cell lung cancer was recently reported (Antonia, Scott et al., 2016, Safety and antitumour activity of durvalumab plus tremelimumab in non-small cell lung cancer: a multicentre, phase 1b study; Lancet Oncol. 2016 Feb. 5. pii: S1470-2045(15)00544-6. doi: 10.1016/S1470-2045(15)00544-6. [Epub ahead of print]). Accordingly, preferred combinations of immune checkpoint modulators of the PD-1 pathway and of the CTLA-4 pathway are (i) Nivolumab (anti-PD1) and Ipilimumab (anti-CTLA4) or (ii) Durvalumab (MEDI4736; anti-PD-L1) and Tremelimumab (anti-CTLA4). Combinations thereof, e.g. Nivolumab (anti-PD1) and Tremelimumab (anti-CTLA4) or Durvalumab (MEDI4736; anti-PD-L1) and Ipilimumab (anti-CTLA4) are also preferred.

Other preferred combinations of at least two distinct immune checkpoint modulators in the context of the present invention may comprise a combination selected from (i) a combination of a KIR inhibitor and a CTLA-4 inhibitor, such as Lirilumab/Ipilimumab; (ii) a combination of a KIR inhibitor and an inhibitor of the PD-1 pathway, such as a PD-1 inhibitor, for example Lirilumab/Nivolumab; (iii) a combination of a LAG3 inhibitor and an inhibitor of the PD-1 pathway, such as a PD-1 inhibitor or a PD-L1 inhibitor, for example as described in Woo et al., 2012, Cancer Res. 72: 917-27 or in Butler N. S. et al., 2011, Nat Immunol. 13: 188-95) and preferred examples of such a combination include Novilumab/BMS-986016 and PDR001/LAG525; (iv) a combination of checkpoint modulators targeting ICOS and an inhibitor of the CTLA-4, for example as described in Fu et al., 2011, Cancer Res. 71: 5445-54; (v) a combination of checkpoint modulators modulating 4-1BB and inhibitor of CTLA-4, such as described in Curran et al., 2011, PLoS One 6(4): el 9499); (vi) a combination of checkpoint modulators targeting PD1 and CD27, such as Novilumab/Varlilumab and Atezolizumab/Varlilumab; (vii) a combination of checkpoint modulators targeting OX40 and CTLA-4, such as MEDI6469/Tremelimumab; (viii) a combination of checkpoint modulators targeting OX40 and PD-1, such as MEDI6469/MEDI4736, MOXR0916/MPDL3280A, MEDI6383/MEDI4736 and GSK3174998/Pembrolizumab; (ix) a combination of checkpoint modulators targeting PD-1 and 4-1B13B, such as Novilumab/Urelumab, Pembrolizumab/PF-05082566 and Avelumab/PF-05082566; (x) a combination of checkpoint modulators targeting PD-1 and IDO, such as Ipilimumab/Indoximod, Pembrolizumab/INCB024360, MEDI4736/INCB024360, MPDL3280A/GDC-0919 and Atezolizumab/INCB024360; (xi) a combination of checkpoint modulators targeting PD-1 and CSF1R, such as Pembrolizumab/PLX3397, Novilumab/FPA008 and MPDL3280A/RO5509554; (xii) a combination of checkpoint modulators targeting PD-1 and GITR, such as Novilumab/BMS-986156 and Pembrolizumab/MK-4166; (xiii) a combination of checkpoint modulators targeting PD-1 and CD40, such as MPDL3280A/RO7009789; (xiv) a combination of checkpoint modulators targeting PD-1 and B7-H3, such as Pembrolizumab/MGA271; (xv) a combination of checkpoint modulators targeting CTLA-4 and B7-H3, such as Ipilimumab/MGA271 and (xvi) a combination of checkpoint modulators targeting KIR and 4-1BB, such as Lirilumab/Urelumab.

Most preferably, the combination of the immune checkpoint modulator and the T-cell redirecting, multifunctional antibody, or fragment thereof, for use according to the present invention comprises at least (a) an inhibitor of CTLA-4 and (0) an inhibitor of PD-1, PD-L1 and/or PD-L2, preferably at least (a) an inhibitor of CTLA-4 and (0) an inhibitor of PD-1. Examples of such a preferred combination include a combination of Yervoy® (Ipilimumab; Bristol Myers Squibb) and Opdivo® (Nivolumab; Bristol Myers Squibb), a combination of Yervoy® (Ipilimumab; Bristol Myers Squibb) and Keytruda® (Pembrolizumab; Merck), a combination of Yervoy® (Ipilimumab; Bristol Myers Squibb) and Durvalumab (MedImmune/AstraZeneca), a combination of Yervoy® (Ipilimumab; Bristol Myers Squibb) and MEDI4736 (AstraZeneca; cf. WO 2011/066389 A1), a combination of Yervoy® (Ipilimumab; Bristol Myers Squibb) and MPDL3280A (Roche/Genentech; cf. U.S. Pat. No. 8,217,149 B2), a combination of Yervoy® (Ipilimumab; Bristol Myers Squibb) and Pidilizumab (CT-011; CureTech), a combination of Yervoy® (Ipilimumab; Bristol Myers Squibb) and MEDI0680 (AMP-514; AstraZeneca), a combination of Yervoy® (Ipilimumab; Bristol Myers Squibb) and MSB-0010718C (Merck), a combination of Yervoy® (Ipilimumab; Bristol Myers Squibb) and MIH1 (Affymetrix), a combination of Yervoy® (Ipilimumab; Bristol Myers Squibb) and AMP-224, a combination of Yervoy® (Ipilimumab; Bristol Myers Squibb) and Lambrolizumab, a combination of Tremelimumab (Pfizer/Medimmune) and Opdivo® (Nivolumab; Bristol Myers Squibb), a combination of Tremelimumab (Pfizer/MedImmune) and Keytruda® (Pembrolizumab; Merck), a combination of Tremelimumab (Pfizer/MedImmune) and Durvalumab (MedImmune/AstraZeneca), a combination of Tremelimumab (Pfizer/MedImmune) and MEDI4736 (AstraZeneca; cf. WO 2011/066389 A1), a combination of Tremelimumab (Pfizer/MedImmune) and MPDL3280A (Roche/Genentech; cf. U.S. Pat. No. 8,217,149 B2), a combination of Tremelimumab (Pfizer/MedImmune) and Pidilizumab (CT-011; CureTech), a combination of Tremelimumab (Pfizer/MedImmune) and MEDI0680 (AMP-514; AstraZeneca), a combination of Tremelimumab (Pfizer/MedImmune) and MSB-0010718C (Merck), a combination of Tremehmumab Pfizer/MedImmune) and MIH1 (Affymetrix), a combination of Tremelimumab (Pfizer/MedImmune) and AMP-224 and a combination of Tremelimumab (Pfizer/MedImmune) and Lambrolizumab.

In the context of the present invention it is also preferred if more than one immune checkpoint modulator (e.g., checkpoint inhibitor) of the same checkpoint pathway is used, in particular at least 2, 3, 4, 5, 6, 7, 8, 9 or 10 immune checkpoint modulators (e.g., checkpoint inhibitors) of the same checkpoint pathway are used, preferably 2, 3, 4 or 5 immune checkpoint modulators (e.g., checkpoint inhibitors) of the same checkpoint pathway are used, more preferably 2, 3 or 4 immune checkpoint modulators (e.g., checkpoint inhibitors) of the same checkpoint pathway are used, even more preferably 2 or 3 immune checkpoint modulators (e.g., checkpoint inhibitors) of the same checkpoint pathway are used and most preferably 2 immune checkpoint modulators (e.g., checkpoint inhibitors) of the same checkpoint pathway are used. Preferred checkpoint pathways to be modulated are the PD-1 pathway or the CTLA-4 pathway. For example, a combination of MEDI4736 and MEDI0680 may be used to modulate, in particular to inhibit, the PD-1 pathway.

In the context of the present invention immune checkpoint modulators may be any kind of molecule or agent, as long as it totally or partially reduces, inhibits, interferes with, activates, stimulates, increases, reinforces or supports the function of one or more checkpoint molecules as described above. In particular, the immune checkpoint modulator binds to one or more checkpoint molecules, such as checkpoint proteins, or to its precursors, e.g. on DNA- or RNA-level, thereby modulating (e.g., totally or partially reducing, inhibiting, interfering with, activating, stimulating, increasing, reinforcing or supporting) the function of one or more checkpoint molecules as described above. Preferred immune checkpoint modulators are oligonucleotides, siRNA, shRNA, ribozymes, anti-sense RNA molecules, immunotoxins, small molecule inhibitors and antibodies or antigen binding fragments thereof (e.g., checkpoint molecule blocking antibodies or antibody fragments, antagonist antibodies or antibody fragments or agonist antibodies or antibody fragments).

Preferably, the immune checkpoint modulator is an oligonucleotide. Such an oligonucleotide is preferably used to decrease protein expression, in particular to decrease the expression of a checkpoint protein, such as the checkpoint receptors or ligands described above. Oligonucleotides are short DNA or RNA molecules, typically comprising from 2 to 50 nucleotides, preferably from 3 to 40 nucleotides, more preferably from 4 to 30 nucleotides and even more preferably from 5 to 25 nucleotides, such as, for example 4, 5, 6, 7, 8, 9 or 10 nucleotides. Oligonucleotides are usually made in the laboratory by solid-phase chemical synthesis. Oligonucleotides maybe single-stranded or double-stranded, however, in the context of the present invention the oligonucleotide is preferably single-stranded. More preferably, the checkpoint modulator oligonucleotide is an antisense-oligonucleotide. Antisense-oligonucleotides are single strands of DNA or RNA that are complementary to a chosen sequence, in particular to a sequence chosen from the DNA or RNA sequence (or a fragment thereof) of a checkpoint protein. Antisense RNA is typically used to prevent protein translation of messenger RNA strands, e.g. of mRNA for a checkpoint protein, by binding to the mRNA. Antisense DNA is typically used to target a specific, complementary (coding or non-coding) RNA. If binding takes place, such a DNA/RNA hybrid can be degraded by the enzyme RNase H. Moreover, morpholino-antisense oligonucleotides can be used for gene knockdowns in vertebrates. For example, Kryczek et al., 2006 (Kryczek I, Zou L, Rodriguez P, Zhu G, Wei S, Mottram P, et al. B7-H4 expression identifies a novel suppressive macrophage population in human ovarian carcinoma. J Exp Med. 2006; 203:871-81) designed a B7-H4-specific morpholino that specifically blocked B7-H4 expression in macrophages, resulting in increased T-cell proliferation and reduced tumor volumes in mice with tumor associated antigen (TAA)-specific T cells.

Preferably, the immune checkpoint modulator is an siRNA. Small interfering RNA (siRNA), sometimes known as short interfering RNA or silencing RNA, is a class of double-stranded RNA molecules, which is typically 20-25 base pairs in length. In the RNA interference (RNAi) pathway, siRNA interferes with the expression of specific genes, such as genes coding for checkpoint proteins, with complementary nucleotide sequences. siRNA functions by causing mRNA to be broken down after transcription, resulting in no translation. Transfection of exogenous siRNA may be used for gene knockdown, however, the effect maybe only transient, especially in rapidly dividing cells. This may be overcome, for example, by RNA modification or by using an expression vector for the siRNA. The siRNA sequence may also be modified to introduce a short loop between the two strands. The resulting transcript is a short hairpin RNA (shRNA, also “small hairpin RNA”), which can be processed into a functional siRNA by Dicer in its usual fashion. shRNA is an advantageous mediator of RNAi in that it has a relatively low rate of degradation and turnover. Accordingly, the immune checkpoint modulator is preferably an shRNA. shRNA typically requires the use of an expression vector, e.g. a plasmid or a viral or bacterial vector.

Preferably, the immune checkpoint modulator is an immunotoxin. Immunotoxins are chimeric proteins that contain a targeting moiety (such as an antibody), which is typically targeting an antigen on a certain cell, such as a cancer cell, linked to a toxin. In the context of the present invention, an immunotoxin comprising a targeting moiety, which targets a checkpoint molecule, is preferred. When the immunotoxin binds to a cell carrying the antigen, e.g. the checkpoint molecule, it is taken in through endocytosis, and the toxin can then kill the cell. Immunotoxins preferably comprise a (modified) antibody or antibody fragment, linked to a (fragment of a) toxin. For linkage, methods are well known in the art. The targeting portion of the immunotoxin typically comprises a Fab portion of an antibody that targets a specific cell type. The toxin is usually cytotoxic, such as a protein derived from a bacterial or plant protein, from which the natural binding domain has been removed so that the targeting moiety of the immunotoxin directs the toxin to the antigen on the target cell. However, immunotoxins can also comprise a targeting moiety other than an antibody or antibody fragment, such as a growth factor. For example, recombinant fusion proteins containing a toxin and a growth factor are also referred to as recombinant immunotoxins.

Preferably, the immune checkpoint modulator is a small molecule drug (also referred to as “small molecule inhibitor”). A small molecule drug is a low molecular weight (up to 900 daltons) organic compound that typically interacts with (the regulation of) a biological process. In the context of the present invention, a small molecule drug which is an immune checkpoint modulator, is an organic compound having a molecular weight of no more than 900 daltons, which totally or partially reduces, inhibits, interferes with, or negatively modulates the function of one or more checkpoint molecules as described above. The upper molecular weight limit of 900 daltons allows for the possibility to rapidly diffuse across cell membranes and for oral bioavailability. More preferably, the molecular weight of the small molecule drug which is an immune checkpoint modulator, is no more than 500 daltons. For example, various A2AR antagonists known in the art are organic compounds having a molecular weight below 500 daltons.

Most preferably, the immune checkpoint modulator is an antibody or an antigen-binding fragment thereof. Such immune checkpoint modulator antibodies or an antigen-binding fragments thereof include in particular antibodies, or antigen binding fragments thereof, that bind to immune checkpoint receptors or antibodies that bind to immune checkpoint receptor ligands. Preferably, immune checkpoint modulator antibodies or an antigen-binding fragments thereof are agonists or antagonists of immune checkpoint receptors or of immune checkpoint receptor ligands. Examples of antibody-type checkpoint modulators include immune checkpoint modulators, which are currently approved as described above, namely, Yervoy® (Ipilimumab; Bristol Myers Squibb), Opdivo® (Nivolumab; Bristol Myers Squibb) and Keytruda® (Pembrolizumab; Merck) and further anti-checkpoint receptor antibodies or anti-checkpoint ligand antibodies as described above.

Preferably, the immune checkpoint modulators in the combination used according to the present invention are antibodies or antigen-binding fragments that can partially or totally block the PD-1 pathway (e.g., they can be partial or full antagonists of the PD-1 pathway), in particular PD-1, PD-L1 or PD-L2, more preferably, the antibody can partially or totally block PD-1 (e.g., they can be partial or full antagonists of PD-1). Such antibodies or antigen-binding fragments include anti-PD-1 antibodies, human anti-PD-1 antibodies, mouse anti-PD-1 antibodies, mammalian anti-PD-1 antibodies, humanized anti-PD-1 antibodies, monoclonal anti-PD-1 antibodies, polyclonal anti-PD-1 antibodies, chimeric anti-PD-1 antibodies, anti-PD-L1 antibodies, anti-PD-L2 antibodies, anti-PD-1 adnectins, anti-PD-1 domain antibodies, single chain anti-PD-1 fragments, heavy chain anti-PD-1 fragments, and light chain anti-PD-1 fragments. For example, the anti-PD-1 antibody may be an antigen-binding fragment. Preferably, the anti-PD-1 antibody is able to bind to human PD-1 and to partially or totally block the activity of (human) PD-1 (e.g., they can be partial or full antagonists of PD-1), thereby in particular unleashing the function of immune cells expressing PD-1.

Preferably, the immune checkpoint modulators in the combination used according to the present invention are antibodies or antigen-binding fragments that can partially or totally block the CTLA-4 pathway (e.g., they can be partial or full antagonists of the CTLA-4 pathway). Such antibodies or antigen-binding fragments include anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, and light chain anti-CTLA4 fragments. For example, the anti-CTLA4 antibody may be an antigen-binding fragment. Preferably, the anti-CTLA4 antibody is able to bind to human CTLA4 and to partially or totally block the activity of CTLA4 (e.g., they can be partial or full antagonists of CTLA-4), thereby in particular unleashing the function of immune cells expressing CTLA4.

Preferred Combinations of a Preferred Immune Checkpoint Modulator and a Preferred T-Cell Redirecting Multifunctional Antibody

As described above, a preferred combination for use according to the present invention comprises a preferred immune checkpoint modulator as described herein. Moreover, a preferred combination for use according to the present invention comprises a preferred 1-cell redirecting multifunctional antibody, or an antigen-binding fragment, as described herein comprising a (preferred) specificity against a T cell surface antigen, a (preferred) specificity against a cancer- and/or tumor-associated antigen and a (preferred) binding site for human FcγRI, FcγRIIa and/or FcγRIII.

A more preferred combination for use according to the present invention comprises (i) a preferred immune checkpoint modulator as described herein and (ii) a preferred T-cell redirecting multifunctional antibody, or an antigen-binding fragment, as described herein comprising a (preferred) specificity against a T cell surface antigen, a (preferred) specificity against a cancer- and/or tumor-associated antigen and a (preferred) binding site for human FcγRI, FcγRIIa and/or FcγRIII. In the following preferred embodiments of a preferred combination for use according to the present invention are described.

In a preferred combination for use according to the present invention the T-cell redirecting multifunctional antibody is a trifunctional bispecific IgG-type antibody wherein
a) the specificity against a T cell surface antigen is a specificity (binding site) for (human) CD3;
b) the specificity against a cancer- and/or tumor-associated antigen is a specificity (binding site) for EpCAM, HER2/neu, GD2, or CD20; and
c) the binding site for human FcγRI, FcγRIIa and/or Fc-RIII is a mouse IgG2a/rat IgG2b Fc region.

More preferably, the T-cell redirecting multifunctional antibody is catumaxomab and/or ektomab.

It is also preferred in the combination for use according to the present invention that the immune checkpoint modulator is an inhibitor of CTLA-4, PD-1, PD-L1 and/or PD-L2 and more preferably the immune checkpoint modulator is an inhibitor of CTLA-4 and/or PD-1. Most preferably, the immune checkpoint modulator is an inhibitor of CTLA-4.

For example, a preferred combination for use according to the present invention comprises
a trifunctional bispecific IgG-type antibody having
a) a specificity (binding site) for (human) CD3;
b) a specificity (binding site) for EpCAM, HER2/neu, GD2, or CD20; and
c) a mouse IgG2a/rat IgG2b Fc region; and
an inhibitor of CTLA-4, PD-L1, PD-L2 and/or PD-1, preferably an inhibitor of CTLA-4 and/or PD-1.

Another preferred example of a combination for use according to the present invention comprises
a trifunctional bispecific IgG-type antibody having
a) a specificity (binding site) for (human) CD3;
b) a specificity (binding site) for EpCAM, HER2/neu, or GD2; and
c) a mouse IgG2a/rat IgG2b Fc region; and
an inhibitor of CTLA-4, PD-L1, PD L2 and/or PD-1, preferably an inhibitor of CTLA-4 and/or PD-1.

Most preferably, the combination for use according to the present invention comprises (i) catumaxomab or ektomab and (ii) an inhibitor of CTLA-4.

Use in Therapeutic Treatment of a Cancer Disease

The combination of the immune checkpoint modulator as described herein and of the T-cell redirecting multifunctional antibody, or antigen-binding fragment thereof, as described herein is for use in therapeutic treatment of a cancer disease.

Such a combination of the immune checkpoint modulator as described herein and of T-cell redirecting multifunctional antibody, or antigen-binding fragment thereof, as described herein is able to initiate or enhance the efficacy of checkpoint modulators, in particular in therapeutic settings, as shown by the present examples.

As used herein, “therapeutic treatment” refers to treatment after the onset of a disease. In particular, “therapeutic treatment” does not include preventive measures applied before the onset of a disease. Since the onset of a disease is often associated with symptom(s) of the disease, human or animal subjects are often “therapeutically” treated after the diagnosis or at least a (strong) assumption that the subject suffers from a certain disease. Therapeutic treatment aims in particular at (i) ameliorating, improving, or curing a disease (state) or (ii) at inhibiting or delaying the progression of a disease (for example, by increasing the average survival time for cancer patients). However, prevention of the onset of a disease cannot typically be achieved by therapeutic treatment.

The combination as described herein is for use (for the preparation of a medicament) for the therapeutic treatment of a cancer disease. The term “disease” as used in the context of the present invention is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

Cancer diseases are a group of diseases involving abnormal cell growth, in particular with the potential to invade or spread to other parts of the body. Cancerous cells/tissue may typically show the six hallmarks of cancer, namely (i) cell growth and division absent the proper signal; (ii) continuous growth and division even given contrary signals, (iii) avoidance of programmed cell death; (iv) limitless number of cell divisions; (v) promoting blood vessel construction; and (vi) invasion of tissue and formation of metastases.

Cancer diseases include diseases caused by defective apoptosis. The cancer may be a solid tumor, blood cancer, or lymphatic cancer. In particular, the cancer may be benign, malign and/or metastatic.

Preferably, in the therapeutic treatment of cancer disease, the combination for use according to the present invention inhibits/delays the ongoing/further growth of a tumor (or of metastases) or decreases the size of the tumor (or the number of metastases) or prevents the reoccurrence of the tumor and/or metastases.

Preferred examples of cancer diseases are preferably selected from acusticus neurinoma, anal carcinoma, astrocytoma, basalioma, Behcet's syndrome, bladder cancer, blastomas, bone cancer, brain metastases, brain tumors, brain cancer (glioblastomas), breast cancer (mamma carcinoma), Burkitt's lymphoma, carcinoids, cervical cancer, colon carcinoma, colorectal cancer, corpus carcinoma, craniopharyngeomas, CUP syndrome, endometrial carcinoma, gall bladder cancer, genital tumors, including cancers of the genitourinary tract, glioblastoma, gliomas, head/neck tumors, hepatomas, histocytic lymphoma, Hodgkin's syndromes or lymphomas and non-Hodgkin's lymphomas, hypophysis tumor, intestinal cancer, including tumors of the small intestine, and gastrointestinal tumors, Kaposi's sarcoma, kidney cancer, kidney carcinomas, laryngeal cancer or larynx cancer, leukemia, including acute myeloid leukaemia (AML), erythroleukemia, acute lymphoid leukaemia (ALL), chronic myeloid leukaemia (CML), and chronic lymphocytic leukaemia (CLL), lid tumor, liver cancer, liver metastases, lung carcinomas (=lung cancer=bronchial carcinoma), small cell lung carcinomas and non-small cell lung carcinomas, and lung adenocarcinoma, lymphomas, lymphatic cancer, malignant melanomas, mammary carcinomas (=breast cancer), medulloblastomas, melanomas, meningiomas, Mycosis fungoides, neoplastic diseases neurinoma, oesophageal cancer, oesophageal carcinoma (=oesophageal cancer), oligodendroglioma, ovarian cancer (=ovarian carcinoma), ovarian carcinoma, pancreatic carcinoma (=pancreatic cancer), penile cancer, penis cancer, pharyngeal cancer, pituitary tumour, plasmocytoma, prostate cancer (=prostate tumors), rectal carcinoma, rectal tumors, renal cancer, renal carcinomas, retinoblastoma, sarcomas, Schneeberger's disease, skin cancer, e.g. melanoma or non-melanoma skin cancer, including basal cell and squamous cell carcinomas as well as psoriasis, pemphigus vulgaris, soft tissue tumours, spinalioma, stomach cancer, testicular cancer, throat cancer, thymoma, thyroid carcinoma, tongue cancer, urethral cancer, uterine cancer, vaginal cancer, various virus-induced tumors such as, for example, papillorna virus-induced carcinomas (e.g. cervical carcinoma=cervical cancer), adenocarcinomas, herpes virus-induced tumors (e.g. Burkitt's lymphoma, EBV-induced B-cell lymphoma, cervix carcinoma), heptatitis B-induced tumors (hepatocell carcinomas), HTLV-1- and HTLV-2-induced lymphomas, vulval cancer, wart conditions or involvement, etc.

Further preferred examples of cancers to be treated with the combination of the immune checkpoint modulator as described herein and of the T-cell redirecting multifunctional antibody, or the fragment thereof, as described herein include brain cancer, prostate cancer, breast cancer, ovarian cancer, esophageal cancer, lung cancer, liver cancer, kidney cancer, melanoma, gut carcinoma, lung carcinoma, head and neck squamous cell carcinoma, Hodgkin's lymphoma, chronic myeloid leukemia, colorectal carcinoma, gastric carcinoma, endometrial carcinoma, myeloid leukemia, lung squamous cell carcinoma, acute lymphoblastic leukemia, acute myelogenous leukemia, bladder tumor, promyelocytic leukemia, non-small cell lung carcinoma, plasmocytoma, and sarcoma.

More preferably, the cancer disease is selected from lung cancer, gastric cancer, ovarian cancer, breast cancer, melanoma, prostate cancer, head and neck squamous cell carcinoma, Hodgkin's lymphoma, non-Hodgkin's lymphomas, bladder tumor, plasmocytoma, and/or sarcoma.

In general, a “combination” of the immune checkpoint modulator as described herein and of the T-cell redirecting multifunctional antibody, or the fragment thereof, as described herein means that the therapy with the immune checkpoint modulator as described herein is combined with the therapy with the T-cell redirecting multifunctional antibody, or the fragment thereof, as described herein. In other words, even if one component (the checkpoint modulator or the T-cell redirecting multifunctional antibody) is not administered, e.g., at the same day as the other component (the other of checkpoint modulator or T-cell redirecting multifunctional antibody), their treatment schedules are intertwined. This means that “a combination” in the context of the present invention does in particular not include the start of a therapy with one component (the checkpoint modulator or the T-cell redirecting multifunctional antibody) after the therapy with the other component (the other of checkpoint modulator or T-cell redirecting multifunctional antibody) is finished. In more general, an “intertwined” treatment schedule of the checkpoint modulator and the T-cell redirecting multifunctional antibody—and, thus, a combination of the checkpoint modulator and the T-cell redirecting multifunctional antibody—means that:
(i) not every administration of the checkpoint modulator (and therefore the complete checkpoint modulator therapy) is completed for more than one week (preferably for more than 3 days, more preferably for more than 2 days, even more preferably for more than a day) before the first administration of the T-cell redirecting multifunctional antibody (and therefore the complete therapy with the T-cell redirecting multifunctional antibody) starts; or
(ii) not every administration of the T-cell redirecting multifunctional antibody (and therefore the complete therapy with the T-cell redirecting multifunctional antibody) is completed for more than one week (preferably for more than 3 days, more preferably for more than 2 days, even more preferably for more than a day) before the first administration of the checkpoint modulator (and therefore the complete checkpoint modulator therapy) starts.

For example, in the combination of the immune checkpoint modulator as described herein and of the T-cell redirecting multifunctional antibody as described herein for use according to the present invention, one component (the checkpoint modulator or the T-cell redirecting multifunctional antibody) may be administered once a week and the other component (the other of checkpoint modulator or T-cell redirecting multifunctional antibody) may be administered once a month. To achieve in this example “a combination” in the sense of the present invention the monthly administered component is to be administered at least once in the same week, in which also the weekly administered other component is administered.

As outlined above, the administration of the immune checkpoint modulator and/or of the T-cell redirecting multifunctional antibody comprised by the combination for use according to the present invention may require multiple successive administrations, e.g. multiple injections. Thus, the administration may be repeated at least two times, for example once as primary immunization injections and, later, as booster injections.

In particular, the immune checkpoint modulator and/or the T-cell redirecting multifunctional antibody comprised by the combination for use according to the present invention may be administered repeatedly or continuously. The immune checkpoint modulator and/or the T-cell redirecting multifunctional antibody comprised by the combination for use according to the present invention may be administered repeatedly or continuously for a period of at least 1, 2, 3, or 4 weeks; 2, 3, 4, 5, 6, 8, 10, or 12 months; or 2, 3, 4, or 5 years. For example, the immune checkpoint modulator comprised by the combination for use according to the present invention may be administered twice per day, once per day, every two days, every three days, once per week, every two weeks, every three weeks, once per month or every two months. For example, the T-cell redirecting multifunctional antibody comprised by the combination for use according to the present invention may be administered twice per day, once per day, every two days, every three days, once per week, every two weeks, every three weeks, once per month or every two months.

Preferably, the T-cell redirecting multifunctional antibody, or the antigen binding fragment thereof, is administered according to an escalating dosage regimen. In general, an “escalating dosage regimen” refers to repeated administration of the antibody, wherein the initial dose (i.e., the single dose of the first administration of the antibody, e.g. in general or relating to one single treatment cycle) is lower than the final dose (i.e., the single dose of the final administration of the antibody, e.g. in general or relating to one single treatment cycle). In particular, the dosage of the antibody increases over the repeated antibody administrations of an escalating dosage regimen. In particular, an escalating dosage regimen comprises two or more distinct “dose levels”, which are administered in an increasing manner, i.e. starting with the lowest dose level, followed by the next higher dose level, optionally followed by the next higher dose level, etc. The term “dose level” refers to a certain dose/amount of the antibody. For example, a dose level of “10 μg” means that a single dose of 10 μg of the antibody is administered once or repeatedly (e.g., two or three times) until the next higher dose level (e.g., a single dose of 50 μg of the antibody administered once or repeatedly) starts. Accordingly, at each dose level one or more (for example, two or three) single doses may be administered. If more than one single dose is administered at a (single) dose level, this means that the single doses (of that dose level) are the same, i.e. the amount of antibody administered at each single dose of a (single) dose level is the same. Accordingly, in an escalating dosage regimen, except for the initial dose, i.e. the first antibody administration, in each single antibody administration the single dose administered is either higher than that of the preceding antibody administration (to enter the next higher dose level) or the same as that of the preceding antibody administration (to maintain the “actual” dose level). Thereby, the term “preceding administration” refers to the (antibody) administration directly preceding the (antibody) administration in question. For example, for the third (antibody) administration the “preceding administration” is the second (antibody) administration, but not the first (antibody) administration; or for the fourth (antibody) administration the “preceding administration” is the third (antibody) administration, but not the first (antibody) administration or the second (antibody) administration. For example, in an escalating dosage regimen (i) the initial dose may be the lowest dose and at each subsequent (antibody) administration the single dose may be higher than in the respective preceding (antibody) administration, such that only one single dose is administered at each dose level; or (ii) at one or more (but not all) of the (antibody) administrations the single doses may be the same as in the respective preceding (antibody) administration (such that more than one single dose is administered at one or more of the dose levels, e.g. at each dose level).

Preferably, the combination for use according to the present invention is administered in one or more treatment cycles. In the context of the present invention, a treatment cycle is a course of one or more treatment(s) that may be repeated on a regular schedule with periods of rest in between. For example, combination for use according to the present invention may be administered in one treatment cycle (e.g., one single dose or repeated doses) and, thereafter, it may be observed whether the cancer or tumor recurs. In particular when the cancer/tumor recurs, a further treatment cycle may be performed. However, a further treatment cycle may also be performed as a prophylactic measure. In particular, the interval between two treatments (e.g., between two single doses of the antibody and/or between two single doses of the checkpoint modulator) within one treatment cycle does preferably not exceed one month (31 days), more preferably it does not exceed 3 weeks, whereas the interval between the end of one treatment cycle and the beginning of the next treatment cycle (in particular relating to the administration of the antibody and/or of the immune checkpoint modulator) is preferably at least one month, preferably at least two months, more preferably at least 3 months even more preferably at least 4 months and most preferably at least 6 months. In other words, the interval between two treatments/administrations (of the antibody and/or checkpoint modulator) within one treatments cycle is preferably less than one month (e.g., no more than two or three weeks), whereas the interval between two treatment cycles (relating to the administration of the antibody and/or checkpoint modulator) is preferably more than one month (e.g., at least two or three months).

Preferably, one treatment cycle comprises (i) one single administration or (ii) one initial dose (first administration) and one or more subsequent administration(s) of the antibody and/or the immune checkpoint modulator. The patient may be subjected to one single or various treatment cycles. Each treatment cycle is typically composed of from 2 to 28, preferably from 2 to 20, more preferably from 3 to 10, and even more preferably from 5 to 8, e.g. 6 or 7, single administrations of the antibody and/or the immune checkpoint modulator.

Preferably, one treatment cycle comprises one or more dose levels. In other words, it is preferred that one treatment cycle comprises (i) repeated administration of the same single doses (one single dose level) or (ii) administration of one or more increasing single doses (wherein at each dose level one or more single doses may be administered as described above). In the latter case, dose levels following upon the initial dose level (the administration(s) of the dose of the first administration) is/are typically higher than the initial dose level. In particular, the initial dose level may preferably comprise only one single administration, i.e. the lowest dose is only administered once, at the very beginning of the treatment/treatment cycle (first administration). In this case the single dose(s) of the one or more subsequent administration(s) is/are higher than the initial dose.

In other words, it is preferred that within a treatment cycle (starting with an initial dose and ending with a final dose), the single dose of each single administration (except the initial dose) is not lower than the preceding dose administered, i.e. each subsequent dose is equal to or higher than the preceding one. More preferably, a treatment cycle follows an escalating dosage regimen as described above. Even more preferably, if more than one treatment cycle is applied, each treatment cycle follows an escalating dosage regimen as described above. Thereby, the dosage regimen of each treatment cycle may be the same or different. As described above, such as escalating dosage regimen may also include doses which are equal to the previous one (i.e. administration of more than one single dose within one (single) dose level). For example, only the initial dose may be lower and all single doses administered subsequently may be the same (and higher than the initial dose) or the initial dose may be lower, the single dose of the second administration may be higher than the initial dose, but lower than all single doses administered subsequently, with all single doses administered subsequently being preferably the same (and higher than the initial dose and the second dose). It is thus preferred that one or more of the single doses administered after the initial dose is/are than the single dose of the preceding administration.

The final dose (level) of a treatment cycle typically reflects the highest single dose of the antibody to be administered within one treatment cycle; i.e. the maximum single dose of the treatment cycle. In particular, at the end of a treatment cycle one, two, three, four, five or more single doses reflecting the maximum single dose may be administered.

In general, the guiding principle for dose escalation is to avoid exposing a patient to sub-therapeutic doses while preserving safety and maintaining rapid accrual. Preferably, within one and the same treatment cycle no single dose is thus lower than the previous one.

Typically, a single treatment cycle includes at least an initial administration and a second administration of the antibody and/or the immune checkpoint inhibitor. In a preferred embodiment a single treatment cycle may include an initial administration, a second administration, a third administration, a fourth administration, a fifth administration and preferably, a sixth administration of the antibody and/or the immune checkpoint inhibitor. Within a single treatment cycle a subsequent administration of the antibody and/or the immune checkpoint inhibitor may be preferably applied 1, 2, 3, 4, 5, 6, 7, 8, 9, 10, 11, 12, 13, 14, 15, 16, 17, 18, 19, 20 or 21 days after the preceding administration, preferably the subsequent administration is applied 2-15 days after the preceding administration, more preferably the subsequent administration is applied 2-10 days after the preceding administration, even more preferably the subsequent administration is applied 3-8 days after the preceding administration.

In the combination of the immune checkpoint modulator as described herein and of the T-cell redirecting multifunctional antibody as described herein for use according to the present invention, the immune checkpoint modulator and the T-cell redirecting multifunctional antibody are preferably administered at about the same time.

“At about the same time”, as used herein, means in particular simultaneous administration or that directly after administration of the immune checkpoint modulator the T-cell redirecting multifunctional antibody is administered or directly after administration of the T-cell redirecting multifunctional antibody the immune checkpoint modulator is administered. The skilled person understands that “directly after” includes the time necessary to prepare the second administration—in particular the time necessary for exposing and disinfecting the location for the second administration as well as appropriate preparation of the “administration device” (e.g., syringe, pump, etc.). Simultaneous administration also includes if the periods of administration of the checkpoint modulator and of the T-cell redirecting multifunctional antibody overlap or if, for example, one component (checkpoint modulator or T-cell redirecting multifunctional antibody) is administered over a longer period of time, such as 30 min, 1 h, 2 h or even more, e.g. by infusion, and the other component (checkpoint modulator or T-cell redirecting multifunctional antibody) is administered at some time during such a long period. Administration of the immune checkpoint modulator and of the T-cell redirecting multifunctional antibody at about the same time is in particular preferred if different routes of administration and/or different administration sites are used.

It is also preferred in the combination of the immune checkpoint modulator as described herein and of the T-cell redirecting multifunctional antibody as described herein for use according to the present invention that the immune checkpoint modulator and the T-cell redirecting multifunctional antibody are administered consecutively. For example, the immune checkpoint modulator is preferably administered before the T-cell redirecting multifunctional antibody. It is also preferred that the immune checkpoint modulator is administered after the T-cell redirecting multifunctional antibody.

In consecutive administration, the time interval between administration of the first component (the checkpoint modulator or the f-cell redirecting multifunctional antibody) and administration of the second component (the other of the checkpoint modulator and the T-cell redirecting multifunctional antibody) is preferably no more than one week, more preferably no more than 3 days, even more preferably no more than 2 days and most preferably no more than 24 h are in between administration of the first component (the checkpoint modulator or the T-cell redirecting multifunctional antibody) and administration of the second component (the other of the checkpoint modulator and the T-cell redirecting multifunctional antibody). It is particularly preferred that the checkpoint modulator and the T-cell redirecting multifunctional antibody are administered at the same day with the time between administration of the first component (the checkpoint modulator of the 1-cell redirecting multifunctional antibody) and administration of the second component (the other of the checkpoint modulator and the T-cell redirecting multifunctional antibody) being preferably no more than 6 hours, more preferably no more than 3 hours, even more preferably no more than 2 hours and most preferably no more than 1 h.

However, it is particularly preferred that the immune checkpoint modulator is administered after the T-cell redirecting multifunctional antibody, or the antigen-binding fragment thereof. More preferably, the immune checkpoint modulator is administered at least 6 hours, preferably at least 12 hours, more preferably at least 18 hours, even more preferably at least 24 hours, still more preferably at least 36 hours and most preferably at least 48 hours after the T-cell redirecting multifunctional antibody, or the antigen-binding fragment thereof. In other words, in a preferred embodiment the interval between the administration of the T-cell redirecting multifunctional antibody, or the antigen-binding fragment thereof, (administered first) and the administration of the immune checkpoint modulator (administered after the antibody) is at least 6 hours, preferably at least 12 hours, more preferably at least 18 hours, even more preferably at least 24 hours, still more preferably at least 36 hours and most preferably at least 48 hours. For example, (in a treatment cycle or in general) the first administration of the immune checkpoint modulator is applied at least 6 hours, preferably at least 12 hours, more preferably at least 18 hours, even more preferably at least 24 hours, still more preferably at least 36 hours and most preferably at least 48 hours after the first administration of the T-cell redirecting multifunctional antibody, or the antigen-binding fragment thereof. As another example, (in a treatment cycle or in general) the final administration of the immune checkpoint modulator is applied at least 6 hours, preferably at least 12 hours, more preferably at least 18 hours, even more preferably at least 24 hours, still more preferably at least 36 hours and most preferably at least 48 hours after the final administration of the T-cell redirecting multifunctional antibody, or the antigen-binding fragment thereof. Most preferably, (in a treatment cycle or in general) each administration of the immune checkpoint modulator is applied at least 6 hours, preferably at least 12 hours, more preferably at least 18 hours, even more preferably at least 24 hours, still more preferably at least 36 hours and most preferably at least 48 hours after each administration of the T-cell redirecting multifunctional antibody, or the antigen-binding fragment thereof. Accordingly, it is particularly preferred that (in a treatment cycle or in general) (i) the first administration of the immune checkpoint modulator is applied at least 6 hours, preferably at least 12 hours, more preferably at least 18 hours, even more preferably at least 24 hours, still more preferably at least 36 hours and most preferably at least 48 hours after the first administration of the T-cell redirecting multifunctional antibody, or the antigen-binding fragment thereof, and (ii) the final administration of the immune checkpoint modulator is applied at least 6 hours, preferably at least 12 hours, more preferably at least 18 hours, even more preferably at least 24 hours, still more preferably at least 36 hours and most preferably at least 48 hours after the final administration of the T-cell redirecting multifunctional antibody, or the antigen-binding fragment thereof.

It is also preferred that the immune checkpoint modulator is administered no more than 96 hours, preferably no more than 84 hours, more preferably no more than 72 hours and most preferably no more than 60 hours after the T-cell redirecting multifunctional antibody, or the antigen-binding fragment thereof. In other words, in a preferred embodiment the interval between the administration of the T-cell redirecting multifunctional antibody, or the antigen-binding fragment thereof, (administered first) and the administration of the immune checkpoint modulator (administered after the antibody) is no more than 96 hours, preferably no more than 84 hours, more preferably no more than 72 hours and most preferably no more than 60 hours. For example, (in a treatment cycle or in general) the first administration of the immune checkpoint modulator is applied no more than 96 hours, preferably no more than 84 hours, more preferably no more than 72 hours and most preferably no more than 60 hours after the first administration of the T-cell redirecting multifunctional antibody, or the antigen-binding fragment thereof. As another example, (in a treatment cycle or in general) the final administration of the immune checkpoint modulator is applied no more than 96 hours, preferably no more than 84 hours, more preferably no more than 72 hours and most preferably no more than 60 hours after the final administration of the T-cell redirecting multifunctional antibody, or the antigen-binding fragment thereof. Most preferably, (in a treatment cycle or in general) each administration of the immune checkpoint modulator is applied no more than 96 hours, preferably no more than 84 hours, more preferably no more than 72 hours and most preferably no more than 60 hours after each administration of the T-cell redirecting multifunctional antibody, or the antigen-binding fragment thereof. Accordingly, it is particularly preferred that (in a treatment cycle or in general) (i) the first administration of the immune checkpoint modulator is applied no more than 96 hours, preferably no more than 84 hours, more preferably no more than 72 hours and most preferably no more than 60 hours after the first administration of the T-cell redirecting multifunctional antibody, or the antigen-binding fragment thereof, and (ii) the final administration of the immune checkpoint modulator is applied no more than 96 hours, preferably no more than 84 hours, more preferably no more than 72 hours and most preferably no more than 60 hours after the final administration of the T-cell redirecting multifunctional antibody, or the antigen-binding fragment thereof.

Most preferably, (in a treatment cycle or in general) (i) the first administration of the immune checkpoint modulator is applied 12-96 hours, preferably 24-84 hours, more preferably 36-72 hours, and most preferably 48-60 hours after the first administration of the T-cell redirecting multifunctional antibody, or the antigen-binding fragment thereof, and/or (ii) the final administration of the immune checkpoint modulator is applied 12-96 hours, preferably 24-84 hours, more preferably 36-72 hours, and most preferably 48-60 hours after the final administration of the T-cell redirecting multifunctional antibody, or the antigen binding fragment thereof.

The present inventors surprisingly found an increased expression of immune checkpoint molecules after T-cell activation by T-cell redirecting multifunctional antibodies as described herein. As shown in Example 2 of the present application, T-cell activation by T-cell redirecting multifunctional antibodies as described herein increased expression of an immune checkpoint molecule, in particular CTLA-4 which is considered the “leader” of the inhibitory immune checkpoints as described above, which peaked at 48-72 hours after antibody administration. In view of those findings the T-cell activation of the T-cell redirecting multifunctional antibodies as described herein, which is crucial in the treatment of cancer and tumor diseases, can be particularly prolonged if the immune checkpoint modulator exerts its actions when the expression of the immune checkpoint molecules are increased or even peak. The above described preferred order of administration (first the antibody, thereafter the immune checkpoint modulator) and terroral intervals are based on those findings and represent administration schedules, which are expected to result in a prolonged activation of T-cells and, thus, increased efficacy in the treatment of cancer and/or tumor diseases. Preferably, the immune checkpoint modulator comprised by the combination for use according to the present invention and the T-cell redirecting multifunctional antibody comprised by the combination for use according to the present invention are administered in a therapeutically effective amount. A “therapeutically effective amount”, as used herein, is the amount which is sufficient for the alleviation of the symptoms of the disease or condition being treated or for inhibiting or delaying the progression of the disease. In other words, a “therapeutically effective amount” means an amount of the T-cell redirecting multifunctional antibody and/or of the checkpoint modulator that is sufficient to significantly induce a positive modification of a disease or disorder, i.e. an amount of the T-cell redirecting multifunctional antibody and/or of the checkpoint modulator, that elicits the biological or medicinal response in a tissue, system, animal or human that is being sought. The term also includes the amount of the T-cell redirecting multifunctional antibody and/or of the immune checkpoint modulator sufficient to reduce the progression of the disease, notably to reduce or inhibit the tumor growth and thereby elicit the (immune) response being sought (i.e. an “inhibition effective amount”). At the same time, however, a “therapeutically effective amount” is preferably small enough to avoid serious side-effects, that is to say to permit a sensible relationship between advantage and risk. The determination of these limits typically lies within the scope of sensible medical judgment. A “therapeutically effective amount” of the T-cell redirecting multifunctional antibody and/or of the checkpoint modulator, will furthermore vary in connection with the particular cancer condition to be treated and also with the age and physical condition of the patient to be treated, the body weight, general health, sex, diet, time of administration, rate of excretion, drug combination, the activity of the specific components (checkpoint modulator and T-cell redirecting multifunctional antibody), the severity of the condition, the duration of the treatment, the nature of the accompanying therapy, of the particular pharmaceutically acceptable carrier used, and similar factors, within the knowledge and experience of the accompanying doctor.

The dosage administered, as single or multiple doses, to an individual will thus vary depending upon a variety of factors, including pharmacokinetic properties, subject conditions and characteristics (sex, age, body weight, health, size), extent of symptoms, concurrent treatments, frequency of treatment and the effect desired.

Preferably, for cancer treatment, the therapeutically effective single dose of the T-cell redirecting multifunctional antibody comprised by the combination for use according to the present invention is from about 0.001 mg to 10 mg, preferably from about 0.01 mg to 5 mg, more preferably from about 0.1 mg to 2 mg per injection or from about 1 nmol to 1 mmol per injection, in particular from 10 nmol to 100 μmol per injection, preferably from 0.1 μmol to 10 μmol per injection. It is also preferred if the therapeutically effective dose of the T-cell redirecting multifunctional antibody comprised by the combination for use according to the present invention is (per kg body weight), in particular for cancer treatment, from about 0.01 μg/kg to 100 μg/kg, preferably from about 0.1 μg/kg to 50 μg/kg, more preferably from about 1 μg/kg to 25 μg/kg, even more preferably from about 2 μg/kg to 20 μg/kg and most preferably from about 2.5 μg/kg to 5 μg/kg.

More preferably, the T-cell redirecting multifunctional antibody, or the antigen binding fragment thereof, as described herein is administered at a single dose in a range of 0.1 to 5000 μg, preferably at a single dose in a range of 1 to 1000 μg, more preferably at a single dose in a range of 2 μg to 750 μg, even more preferably at a single dose in a range of 3 μg to 700 μg, still more preferably at a single dose in a range of 5 μg to 600 μg, and most preferably at a single dose in a range of 10 μg-500 μg.

In the context of the present invention, a “single dose” (or “each dose”) is an individual dose, which is administered to one patient at one administration time.

Most preferably, the T-cell redirecting multifunctional antibody, or the antigen binding fragment thereof, is administered at a single dose of no more than 1 mg, preferably no more than 0.9 mg, more preferably no more than 0.8 mg, even more preferably no more than 0.75 mg, still more preferably no more than 0.6 mg, and most preferably no more than 0.5 mg.

Preferably, the initial dose of the antibody, or the antigen binding fragment thereof, is in a range of 0.5 to 200 μg, preferably 1 to 150 μg, more preferably 2 to 100 μg, most preferably 5 to 70 μg. The initial dose is the single dose of the first administration and preferably the lowest dose of one treatment cycle.

Preferably, the first subsequent dose level of the antibody, or the antigen binding fragment thereof, exceeds the initial dose level (amount administered as initial dose), preferably by a factor of 1.1 to 10.0, more preferably by a factor of 1.2 to 5.0 and even more preferably by a factor of 1.5 to 3.0, and, optionally, the second subsequent dose level and each following subsequent dose level exceeds the initial dose level (amount administered as initial dose) by a factor of 1.1 to 10.0, preferably by a factor of 1.5 to 5.0.

The maximum dose (within a treatment cycle) of the antibody, or the antigen binding fragment thereof, is preferably selected from a range of 25 μg to 1000 μg, preferably from 50 μg to 750 μg, more preferably 75 μg-500 μg.

Preferably, the therapeutically effective dose of the immune checkpoint modulator comprised by the combination for use according to the present invention is (per kg body weight), in particular for cancer treatment, from about 0.01 mg/kg to 100 mg/kg, preferably from about 0.05 mg/kg to 50 mg/kg, more preferably from about 0.1 mg/kg to 25 mg/kg, even more preferably from about 0.5 mg/kg to 15 mg/kg and most preferably from about 1 mg/kg to 10 mg/kg.

The T-cell redirecting multifunctional antibody comprised by the combination for use according to the present invention and the immune checkpoint modulator comprised by the combination for use according to the present invention can be administered by various routes of administration, for example, systemically or locally. Routes for systemic administration in general include, for example, transdermal, oral and parenteral routes, which include subcutaneous, intravenous, intramuscular, intraarterial, intradermal and intraperitoneal routes and/or intranasal administration routes. Routes for local administration in general include, for example, topical administration routes, but also administration directly at the site of affliction, such as intratumoral administration.

Preferably, the T-cell redirecting multifunctional antibody comprised by the combination for use according to the present invention and the immune checkpoint modulator comprised by the combination for use according to the present invention are administered by a parenteral route of administration. More preferably, the T-cell redirecting multifunctional antibody comprised by the combination for use according to the present invention and the immune checkpoint modulator comprised by the combination for use according to the present invention are administered via intravenous, intratumoral, intradermal, subcutaneous, intramuscular, intranasal, or intranodal route. Even more preferably, the T-cell redirecting multifunctional antibody comprised by the combination for use according to the present invention and the immune checkpoint modulator comprised by the combination for use according to the present invention are administered intravenously and/or subcutaneously.

Preferably, the T-cell redirecting multifunctional antibody comprised by the combination for use according to the present invention and the immune checkpoint modulator comprised by the combination for use according to the present invention are administered via the same route of administration, preferably via the same parenteral route of administration, more preferably intravenously or subcutaneously.

However, it is also preferred that the T-cell redirecting multifunctional antibody comprised by the combination for use according to the present invention and the immune checkpoint modulator comprised by the combination for use according to the present invention are administered via distinct routes of administration, preferably via distinct parenteral routes of administration, more preferably the immune checkpoint modulator comprised by the combination for use according to the present invention is administered intravenously and the T-cell redirecting multifunctional antibody comprised by the combination for use according to the present invention is administered via intratumoral, intradermal, subcutaneous, intramuscular, or intranodal route, preferably the T-cell redirecting multifunctional antibody comprised by the combination for use according to the present invention is administered subcutaneously.

The T-cell redirecting multifunctional antibody comprised by the combination for use according to the present invention and the immune checkpoint modulator comprised by the combination for use according to the present invention may be provided in the same or in distinct compositions.

Preferably, the T-cell redirecting multifunctional antibody comprised by the combination for use according to the present invention and the immune checkpoint modulator comprised by the combination for use according to the present invention are provided in distinct compositions. Thereby, different other components, e.g. different vehicles, can be used for the T-cell redirecting multifunctional antibody and for the checkpoint modulator. Moreover, the T-cell redirecting multifunctional antibody and the immune checkpoint modulator can be administered via different routes of administration and the doses (in particular the relation of the doses) can be adjusted according to the actual need.

However, it is also preferred that the immune checkpoint modulator and the T-cell redirecting multifunctional antibody are provided in the same composition. Such a composition comprising both, the immune checkpoint modulator and the T-cell redirecting multifunctional antibody is described in more detail below (“composition according to the present invention”).

No matter whether a composition comprises only the immune checkpoint modulator (and not the T-cell redirecting multifunctional antibody), only the T-cell redirecting multifunctional antibody (and not the checkpoint modulator) or both, such a composition may be a pharmaceutical composition.

In particular, such a composition, which comprises only the immune checkpoint modulator (and not the T-cell redirecting multifunctional antibody), only the T-cell redirecting multifunctional antibody (and not the checkpoint modulator) or both, is preferably a (pharmaceutical) composition which optionally comprises a pharmaceutically acceptable carrier and/or vehicle, or any excipient, buffer, stabilizer or other materials well known to those skilled in the art.

In the context of the present invention, a pharmaceutically acceptable carrier typically includes the liquid or non-liquid basis of the pharmaceutical composition. The term “compatible” as used herein means that these constituents of the pharmaceutical composition are capable of being mixed with the antibody, or the antigen-binding fragment thereof, as defined above in such a manner that no interaction occurs which would substantially reduce the pharmaceutical effectiveness of the pharmaceutical composition under typical use conditions. Pharmaceutically acceptable carriers and vehicles must, of course, have sufficiently high purity and sufficiently low toxicity to make them suitable for administration to a subject to be treated.

Preferably, the pharmaceutical composition is in the form of a lyophilized powder or in the form of a liquid composition, preferably an aqueous solution. Hence, the pharmaceutical composition of the present invention may be provided as a dried, lyophilized powder or, more preferably in solution (dissolved in a vehicle). If provided as lyophilized powder by the manufacturer, it is usually dissolved in an appropriate solution (aqueous solution; such as water for injection or saline, optionally buffered such as PBS) shortly prior to administration. Vials of liquid medication can be single use or multi-use.

In another preferred embodiment, the checkpoint modulator, the T-cell redirecting multifunctional antibody, and/or the pharmaceutical composition (comprising one or both thereof) is not lyophilized. Thus, it is preferred that the checkpoint modulator, the T-cell redirecting multifunctional antibody, and/or the pharmaceutical composition (comprising one or both thereof) is not lyophilized, but provided in a solution, preferably in an aqueous solution, more preferably in an aqueous buffered solution.

It is thus particularly preferred that the pharmaceutical composition is provided in liquid form. Thus, the pharmaceutically acceptable carrier will typically comprise one or more (compatible) pharmaceutically acceptable liquid carriers. Examples of (compatible) pharmaceutically acceptable liquid carriers include pyrogen-free water, isotonic saline or buffered (aqueous) solutions, e.g. citrate buffered solutions; polyols, such as, for example, polypropylene glycol, glycerol, sorbitol, mannitol and polyethylene glycol; alginic acid, further inorganic or organic polymers such as PLGA, preferably to provide a sustained release effect to the present active agent. Preferably, in a liquid pharmaceutical composition the carrier may be pyrogen-free water; isotonic saline or buffered (aqueous) solutions, e.g. phosphate, citrate etc. buffered solutions. Particularly for injection or instillation of the pharmaceutical composition, water or preferably a buffer, more preferably an aqueous buffer, such as citrate buffer, may be used.

Accordingly, it is preferred that the pharmaceutical composition comprises a buffer, preferably an organic acid buffer (i.e. a buffer based on an organic acid), such as citrate buffer, succinate buffer and tartrate buffer, more preferably the pharmaceutical composition comprises a citrate buffer. The organic acid buffer is thus preferably selected from the group consisting of citrate buffer, succinate buffer, tartrate buffer, and phosphate-citrate buffer, more preferably selected from the group consisting of citrate buffer, succinate buffer and tartrate buffer. It is particularly preferred that the buffer is a citrate buffer. In general, a buffer may (also) contain a sodium salt, preferably at least 30 mM of a sodium salt, a calcium salt, preferably at least 0.05 mM of a calcium salt, and/or optionally a potassium salt, preferably at least 1 mM of a potassium salt. The sodium, calcium and/or potassium salts may occur in the form of their halogenides, e.g. chlorides, iodides, or bromides, in the form of their hydroxides, carbonates, hydrogen carbonates, or sulfates, etc. Without being limited thereto, examples of sodium salts include e.g. NaCl, NaI, NaBr, Na₂CO₃, NaHCO₃, Na₂SO₄, examples of the optional potassium salts include e.g. KCl, KI, KBr, K₂CO₃, KHCO₂, K₂SO₁, and examples of calcium salts include e.g. CaCl₂, CaI₂, CaBr₂, CaCO₃, CaSO₄, Ca(OH)₂. Furthermore, organic anions of the aforementioned cations may be contained in the buffer.

The pharmaceutical composition may also comprise saline (0.9% NaCl), Ringer-Lactate solution or PBS (phosphate buffered saline). For example, the pharmaceutical composition may be provided as stock solution of the antibody, or the antigen binding fragment thereof, in an appropriate buffer, such as an organic acid buffer as described above, preferably citrate buffer, and only just before administration that stock solution may be diluted by saline (0.9% NaCl), Ringer-Lactate solution or PBS to achieve the antibody concentration to be administered.

Furthermore, one or more compatible solid or liquid fillers or diluents or encapsulating compounds may be used as well for the pharmaceutical composition, which are suitable for administration to a subject to be treated. Further examples of compounds which may be comprised by the pharmaceutical composition include sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. In addition, preservatives, stabilizers, antioxidants and/or other additives may be included, as required. The pharmaceutical composition may, thus, also comprise stabilizing agents such as Tween® 80 or Tween® 20. Optionally, excipients conferring sustained release properties to the antibody, or the antigen binding fragment thereof, as described herein may also be comprised by the pharmaceutical composition.

In a preferred embodiment, the pharmaceutical composition comprises no further components in addition to (i) the T-ceil redirecting multifunctional antibody, or the antigen binding fragment thereof, as described herein or the immune checkpoint modulator as described herein; (ii) a buffer as described herein; and, optionally, (iii) water for injection, saline and/or PBS.

The compositions, in particular pharmaceutical compositions, as described herein may be adapted for delivery by repeated administration.

Further materials as well as formulation processing techniques and the like, which are useful in the context of compositions, in particular pharmaceutical compositions, or in the context of their preparation are set out in “Part 5 of Remington's “The Science and Practice of Pharmacy”, 22^{nd} Edition, 2012, University of the Sciences in Philadelphia, Lippincott Williams & Wilkins”.

The subject to be treated is preferably a human or non-human animal, in particular a mammal or a human. More preferably, the subject to be treated is preferably a human. Preferably, subjects are patients diagnosed with cancer. For example, young (less than 15 years old) or elderly (more than 60 years old) patients may be treated according to the present invention. For elderly patients, it is of particular advantage to administer the drug by a route which requires a physician, as thereby compliance is ensured. At the same time, the administration should be preferably pain-free.

In general, patients having a cancer disease, irrespective of their age, who are preferably not under immunosuppressive treatment may particularly benefit from the use of the combination of the T-cell redirecting multifunctional antibody and the immune checkpoint inhibitor according to the invention.

Combination with a Glucocorticoid

Preferably, the combination for use according to the present invention as described herein further comprises
(iii) a glucocorticoid.

In other words, a preferred combination according to the present invention comprises
(i) an immune checkpoint modulator;
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb; and
(iii) a glucocorticoid
for use in therapeutic treatment of a cancer disease.

It is understood that preferred embodiments of the immune checkpoint modulator described above, preferred embodiments of the T-cell redirecting multifunctional antibody, or the antigen binding fragment thereof, as described above, as well as preferred embodiments of the combination or use thereof (e.g., regarding the preparation, diseases to be treated, administration etc.) as described above apply accordingly to a combination according to the present invention further comprising a glucocorticoid.

Glucocorticoids (GCs) are a class of corticosteroids, that bind to the glucocorticoid receptor. GCs are part of the feedback mechanism in the immune system which reduces certain aspects of immune function, such as reduction of inflammation. Even though GCs are well-known to interfere with some of the abnormal mechanisms in cancer cells, and, therefore, GCs are used in high doses to treat certain types of cancer (for example lymphomas and leukemias, where GCs exert inhibitory effects on lymphocyte proliferation), in the context of the present invention glucocorticoids are not administered to treat the cancer or tumor disease itself, but to decrease the adverse side effects of the T-cell redirecting multifunctional antibody, or the antigen binding fragment thereof, and/or of the immune checkpoint modulator. Accordingly, in the context of the present invention, the glucocorticoid is neither administered as (i) stand-alone treatment for cancer, nor (ii) as part of chemotherapeutic regimen to treat cancer, such as C-MOPP (cyclophosphamide, vincristine, procarbazine and prednisone), CHOP (cyclophosphamide, doxorubicin, vincristine, and prednisone), m-BACOD (methotrexate, bleomycin, doxorubicin, cyclophosphamide, vincristine, dexamethasone and leucovorin) and MACOP-B (methotrexate, doxorubicin, cyclophosphamide, vincristine, prednisone, bleomycin and leucovorin). In particular, the combination according to the present invention does preferably not comprise a (further) chemotherapeutic agent, such as cyclophosphamide, vincristine, procarbazine, doxorubicin, methotrexate, and bleomycin. Moreover, the cancer disease to be treated with the combination according to the present invention may not include lymphomas and/or leukemias, in particular if the combination according to the present invention comprises a glucocorticoid. In other words, preferably, cancer diseases other than lymphomas and/or leukemias may be treated with the combination according to the present invention, in particular if it comprises a glucocorticoid.

Preferably, the glucocorticoid is selected from the group consisting of prednisone, prednisolone, methylprednisolone, triamcinolone, betamethasone, dexamethasone, cortisone acetate, prednylidene, deflazacort, cloprednole, fluocortolone and budenoside.

Most preferably the glucocorticoid is dexamethasone. Dexamethasone shows very strong glucocorticoid activity, whereas mineralocorticoid activity is essentially absent. Accordingly, dexamethasone is a very potent glucocorticoid. Moreover, dexamethasone is a glucocorticoid with long-lasting effects (biological half-time 36-54 hours) and is, thus, particularly suitable for treatments requiring long-lasting or continuous glucocorticoid activity. Among many other applications, dexamethasone is particularly suitable also for patients suffering from cardiac insufficiency or hypertonia. In addition, the strong antiphlogistical and immune suppressive (anti-allergic) activity of dexamethasone are therapeutically important. Moreover, it is advantageous that after an i.v. injection of dexamethasone the maximum plasma concentration is reached within a few minutes.

Preferably, the glucocorticoid is administered intravenously (i.v.) or orally (p.o.).

The dose of the glucocorticoid is typically selected depending on the type of glucocorticoid used. For dexamethasone, a single dose may preferably be in the range of 1-100 mg, more preferably in the range of 2-80 mg, even more preferably in the range of 5-70 mg, and most preferably in the range of 10-50 mg, such as 10 mg, 20 mg or 40 mg, particularly preferably 10 or 20 mg. For prednisolone or prednisone, the dose will typically be higher due to its lower potency. For example, a single dose of prednisolone or prednisone may preferably be in the range of 50-500 mg, more preferably in the range of 100-400 mg, even more preferably in the range of 150-300 mg, and most preferably in the range of 200-250 mg. Based on the well-known glucocorticoid potency of the various glucocorticoids, the skilled person may easily retrieve similar dose ranges for the other glucocorticoids. For example, betamethasone shows essentially the same glucocorticoid activity as dexamethasone and will, thus, be administered in essentially the same doses, whereas, for example, prednilydene shows essentially the same glucocorticoid activity as prednisone and prednisolone and will, thus, be administered in essentially the same doses.

In general, the glucocorticoid may be administered before the T-cell redirecting multifunctional antibody, or the antigen binding fragment thereof, and/or the immune checkpoint modulator; at about the same time as the T-cell redirecting multifunctional antibody, or the antigen binding fragment thereof, and/or the immune checkpoint modulator; or after the T-cell redirecting multifunctional antibody, or the antigen binding fragment thereof, and/or the immune checkpoint modulator. Preferably, the glucocorticoid is administered before the administration of the T-cell redirecting multifunctional antibody, or the antigen binding fragment thereof, and/or before the administration of the immune checkpoint modulator. Thereby, it is preferred that the glucocorticoid is administered no longer than six hours, preferably no longer than five hours, more preferably no longer than four hours, even more preferably no longer than three hours, still more preferably no longer than two hours and most preferably no longer than one hour before the administration of the T-cell redirecting multifunctional antibody, or the antigen binding fragment thereof, and/or no longer than six hours, preferably no longer than five hours, more preferably no longer than four hours, even more preferably no longer than three hours, still more preferably no longer than two hours and most preferably no longer than one hour before the administration of the immune checkpoint modulator.

For example, in combination with a preferred embodiment of the administration of the T-cell redirecting multifunctional antibody (or the antigen binding fragment thereof) and the immune checkpoint modulator as described above, in a particularly preferred embodiment at first the glucocorticoid is administered, followed by the T-cell redirecting multifunctional antibody (or the antigen binding fragment thereof), which may be administered, for example no longer than one or two hours after glucocorticoid administration and, thereafter, the immune checkpoint modulator is administered, e.g. at least 6 hours, preferably at least 12 hours, more preferably at least 24 hours, even more preferably at least 36 hours and most preferably at least 48 hours after the administration of the T-cell redirecting multifunctional antibody (or the antigen binding fragment thereof. Optionally, the glucocorticoid may be thereby also be administered before administration of the immune checkpoint modulator, for example, no longer than one or two hours before administration of the immune checkpoint modulator.

In treatment schedules requiring repeated administration of the T-cell redirecting multifunctional antibody (or the antigen binding fragment thereof) and/or of the immune checkpoint modulator, the glucocorticoid is preferably administered at least before the dose of the antibody/checkpoint modulator is increased (e.g., at the first administration of each dose level in escalating dosage regimen). More preferably, the glucocorticoid is administered before each administration of the T-cell redirecting multifunctional antibody (or the antigen binding fragment thereof) and/or of the immune checkpoint modulator. Accordingly, the above described particularly preferred embodiment of an administration schedule applies preferably to each antibody/checkpoint modulator administration, including, for example the first and/or the final administration of the T-cell redirecting multifunctional antibody (or the antigen binding fragment thereof) and/or of the immune checkpoint modulator.

It is also preferred that the glucocorticoid is administered at about the same time as the T-cell redirecting multifunctional antibody, or the antigen binding fragment thereof, and/or the glucocorticoid is administered at about the same time the immune checkpoint modulator. Thereby, the phrase “at about the same time” has the same meaning as defined above (which applies throughout the present application).

Kit for Use According to the Present Invention

In a further aspect, the present invention also provides a kit, in particular a kit of parts, comprising
(i) an immune checkpoint modulator and
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb;
for use in therapeutic treatment of a cancer disease, in particular in a human subject.

In particular, such a kit for use according to the present invention comprises (i) the immune checkpoint modulator as described above (in the context of the combination for use according to the present invention) and (ii) the T-cell redirecting multifunctional antibody as described above (in the context of the combination for use according to the present invention). Moreover, such a kit is for use in therapeutic treatment of a cancer disease as described above, in particular in a human subject as described above. In other words, preferred embodiments of the immune checkpoint modulator as described above (in the context of the combination for use according to the present invention) are also preferred in the kit according to the present invention. Accordingly, preferred embodiments of the T-cell redirecting multifunctional antibody as described above (in the context of the combination for use according to the present invention) are also preferred in the kit according to the present invention. Moreover, preferred embodiments of the use in therapeutic treatment of a cancer disease as described above (in the context of the combination for use according to the present invention) are also preferred for the kit according to the present invention.

For example, the immune checkpoint modulator and/or the T-cell redirecting multifunctional antibody, or the antigen-binding fragment thereof, may be provided in the same composition or in distinct compositions, as described above.

Moreover, based on the preferred single doses of the T-cell redirecting multifunctional antibody, or the antigen-binding fragment thereof, as described above, the T-cell redirecting multifunctional antibody, or the antigen-binding fragment thereof, is preferably provided in the kit according to the present invention in single doses, wherein each single dose does not exceed 1 mg, preferably each single dose does not exceed 0.9 mg, more preferably each single dose does not exceed 0.8 mg, even more preferably each single dose does not exceed 0.75 mg and most preferably each single dose does not exceed 0.5 mg.

In addition, the kit according to the present invention preferably comprises
(iii) a glucocorticoid.

In this context again preferred embodiments of the glucocorticoid as described above (in the context of the combination for use according to the present invention) are also preferred for the kit according to the present invention. For example, the glucocorticoid is preferably selected from the group consisting of prednisone, prednisolone, methylprednisolone, triamcinolone, betamethasone, dexamethasone, cortisone acetate, prednylidene, deflazacort, cloprednole, fluocortolone and budenoside, most preferably the glucocorticoid is dexamethasone, as described above.

The various components of the kit may be packaged in one or more containers. The above components may be provided in a lyophilized or dry form or dissolved in a suitable buffer. For example, the kit may comprise a (pharmaceutical) composition comprising the immune checkpoint modulator as described above and a (pharmaceutical) composition comprising the T-cell redirecting multifunctional antibody as described above, e.g. with each composition in a separate container. The kit may also comprise a (pharmaceutical) composition comprising both, the immune checkpoint modulator and the T-cell redirecting multifunctional antibody, as described above.

The kit may also comprise additional reagents including, for instance, buffers for storage and/or reconstitution of the above-referenced components, washing solutions, and the like.

In addition, the kit-of-parts according to the present invention may optionally contain instructions of use. Preferably, the kit further comprises a package insert or label with directions to treat a cancer disease as described herein by using a combination of the immune checkpoint modulator and the T-cell redirecting multifunctional antibody. Optionally, the combination (and, thus, the directions of the package insert or label of the kit) may further include (iii) a glucocorticoid as described above. In particular, the directions to use the combination according to the present invention as described above may include the administration regimen as described above, in particular the preferred embodiments thereof.

Composition for Use According to the Present Invention

In a further aspect, the present invention also provides a composition comprising
(i) an immune checkpoint modulator and
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb.

In particular, such a composition according to the present invention comprises (i) the immune checkpoint modulator as described above (in the context of the combination for use according to the present invention) and (ii) the T-cell redirecting multifunctional antibody as described above (in the context of the combination for use according to the present invention). In other words, preferred embodiments of the immune checkpoint modulator as described above (in the context of the combination for use according to the present invention) are also preferred in the composition according to the present invention. Accordingly, preferred embodiments of the T-cell redirecting multifunctional antibody as described above (in the context of the combination for use according to the present invention) are also preferred in the composition according to the present invention.

Moreover, a composition comprising the immune checkpoint modulator as described above and the T-cell redirecting multifunctional antibody as described above and preferred embodiments of such a composition are described above (in the context of the combination for use according to the present invention). It is understood that the same description, in particular the same preferred embodiments as described above for the composition apply accordingly to the composition as described here.

For example, the composition according to the present invention preferably comprises
(iii) a glucocorticoid.

In this context again preferred embodiments of the glucocorticoid as described above (in the context of the combination for use according to the present invention) are also preferred for the composition according to the present invention. For example, the glucocorticoid is preferably selected from the group consisting of prednisone, prednisolone, methylprednisolone, triamcinolone, betamethasone, dexamethasone, cortisone acetate, prednylidene, deflazacort, cloprednole, fluocortolone and budenoside, most preferably the glucocorticoid is dexamethasone, as described above.

Preferably, the composition is for use in medicine, more preferably, the composition is for use in therapeutic treatment of a cancer disease, in particular in a human subject.

Accordingly, such a composition is for use in therapeutic treatment of a cancer disease as described above, in particular in a human subject as described above. In other words, preferred embodiments of the use in therapeutic treatment of a cancer disease as described above (in the context of the combination for use according to the present invention) are also preferred for the composition according to the present invention.

Accordingly, the composition preferably comprises a pharmaceutically acceptable carrier.

Preferred examples of such a pharmaceutically acceptable carrier are as described above.

It is also preferred that the (pharmaceutical) composition is used in a method for treating a subject, preferably a human subject, who is suffering from a cancer disease.

Method and Combination Therapy According to the Present Invention

In a further aspect, the present invention provides a method for therapeutically treating cancer or initiating, enhancing or prolonging an anti-tumor-response in a subject in need thereof comprising administering to the subject
(i) an immune checkpoint modulator and
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb.

In particular, such a method according to the present invention comprises administration of (i) the immune checkpoint modulator as described above (in the context of the combination for use according to the present invention) and (ii) the T-cell redirecting multifunctional antibody as described above (in the context of the combination for use according to the present invention). Moreover, such a method is useful in therapeutic treatment of a cancer disease as described above, in particular in a human subject as described above. In other words, preferred embodiments of the immune checkpoint modulator as described above (in the context of the combination for use according to the present invention) are also preferred in the method according to the present invention. Accordingly, preferred embodiments of the T-cell redirecting multifunctional antibody as described above (in the context of the combination for use according to the present invention) are also preferred in the method according to the present invention. Moreover, preferred embodiments of the use in therapeutic treatment of a cancer disease as described above (in the context of the combination for use according to the present invention) are also preferred for the method according to the present invention.

For example, the immune checkpoint modulator and/or the T-cell redirecting multifunctional antibody, or the antigen-binding fragment thereof, may be provided in the same composition or in distinct compositions, as described above. As another example, the method according to the present invention preferably comprises administering to the subject
(iii) a glucocorticoid.

In this context again preferred embodiments of the glucocorticoid as described above (in the context of the combination for use according to the present invention) are also preferred for the method according to the present invention. For example, the glucocorticoid is preferably selected from the group consisting of prednisone, prednisolone, methylprednisolone, triamcinolone, betamethasone, dexamethasone, cortisone acetate, prednylidene, deflazacort, cloprednole, fluocortolone and budenoside, most preferably the glucocorticoid is dexamethasone, as described above.

Preferably, the subject is a human subject diagnosed with cancer.

Moreover, preferred embodiments of the administration regimen described above in the context of the combination according to the present invention also apply to the method according to the present invention. Accordingly, (i) the immune checkpoint modulator, and/or (ii) the T-cell redirecting multifunctional antibody, or the antigen binding fragment thereof, and/or, optionally, (iii) the glucocorticoid is/are preferably administered as described above.

In a further aspect, the present invention also provides a method of prolonging T-cell activation in a subject comprising administering to a subject a combination of:
(i) an immune checkpoint modulator and
(ii) a T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb.

As surprisingly found by the present inventors, a T-cell redirecting multifunctional antibody (or an antigen-binding fragment thereof) as defined herein induces increased expression of immune checkpoint molecules, such as CTLA-4 (cf. Example 2, FIG. 2). Namely, as shown in Example 2 of the present application, T-cell activation by T-cell redirecting multifunctional antibodies as described herein increased expression of an immune checkpoint molecule, in particular CTLA-4 which is considered the “leader” of the inhibitory immune checkpoints as described above. In general, the T-cell activation of the T-cell redirecting multifunctional antibodies as described herein is crucial in the treatment of cancer and tumor diseases. Immune checkpoint molecules, such as CTLA-4, counteract the T-cell activation, in particular due to their increased expression. Accordingly, administration of an immune checkpoint modulator prolongs the T-cell activation of the T-cell redirecting multifunctional antibody (or an antigen-binding fragment thereof), since it counteracts the increased expression of the immune checkpoint molecule, which would without immune checkpoint modulator terminate (or at least decrease) antibody-mediated T-cell activation.

In particular, such a method according to the present invention comprises administration of (i) the immune checkpoint modulator as described above (in the context of the combination for use according to the present invention) and (ii) the T-cell redirecting multifunctional antibody as described above (in the context of the combination for use according to the present invention), and, optionally (iii) the glucocorticoid as described above (in the context of the combination for use according to the present invention). In other words, preferred embodiments of the immune checkpoint modulator as described above (in the context of the combination for use according to the present invention) are also preferred in the method according to the present invention. Accordingly, preferred embodiments of the T-cell redirecting multifunctional antibody as described above (in the context of the combination for use according to the present invention) are also preferred in the method according to the present invention. Moreover, preferred embodiments of the use in therapeutic treatment of a cancer disease as described above (in the context of the combination for use according to the present invention) are also preferred for the method according to the present invention (including, for example, as preferred administration regimen).

In a further aspect, the present invention also provides a combination therapy for therapeutically treating cancer, wherein the combination therapy comprises administration of
(i) an immune checkpoint modulator and
(ii) an (isolated) T-cell redirecting multifunctional antibody, or an antigen binding fragment thereof, comprising:
   (a) a specificity against a T cell surface antigen;
   (b) a specificity against a cancer- and/or tumor-associated antigen; and
   (c) a binding site for human FcγRI, FcγRIIa and/or FcγRIII, wherein the antibody, or the antigen binding fragment thereof, binds with a higher affinity to human FcγRI, Fc-RIIa and/or FcγRIII than to human FcγRIIb.

Preferred embodiments of such a combination therapy are preferred embodiments of the T-cell redirecting multifunctional antibody as described above, embodiments of the checkpoint modulator as described above, and/or—in more general—preferred embodiments of the combination for use according to the present invention. The kit according to the present invention and the (pharmaceutical) composition according to the present invention may be used in the method and/or in the combination therapy according to the present invention.

For example, the immune checkpoint modulator and/or the T-cell redirecting multifunctional antibody, or the antigen-binding fragment thereof, may be provided in the same composition or in distinct compositions, as described above. As another example, the combination therapy according to the present invention preferably comprises administering to the subject
(iii) a glucocorticoid.

In this context again preferred embodiments of the glucocorticoid as described above (in the context of the combination for use according to the present invention) are also preferred for the combination therapy according to the present invention. For example, the glucocorticoid is preferably selected from the group consisting of prednisone, prednisolone, methylprednisolone, triamcinolone, betamethasone, dexamethasone, cortisone acetate, prednylidene, deflazacort, cloprednole, fluocortolone and budenoside, most preferably the glucocorticoid is dexamethasone, as described above.

Subjects to be treated with such a combination therapy are the same as described for the combination for use according to the present invention. Preferably, the immune checkpoint modulator and the T-cell redirecting multifunctional antibody, or an antigen binding fragment thereof, are administered to a human subject.

Moreover, preferred embodiments of the administration regimen described above in the context of the combination according to the present invention also apply to the combination therapy according to the present invention. Accordingly, (i) the immune checkpoint modulator, and/or (ii) the T-cell redirecting multifunctional antibody, or the antigen binding fragment thereof, and/or, optionally, (iii) the glucocorticoid is/are preferably administered as described above.

### BRIEF DESCRIPTION OF THE FIGURES

in the following a brief description of the appended figures will be given. The figures are intended to illustrate the present invention in more detail. However, they are not intended to limit the subject matter of the invention in any way.

FIG. 1 shows schematically the assumed mechanisms underlying a therapeutic combination of T-cell redirecting trifunctional antibodies comprising a specificity against a tumor-associated antigen (TAA) and a specificity against a T cell (e.g., CD3) and checkpoint molecule blocking antibodies. T-cells are activated and redirected to the targeted tumor cells by trifunctional antibodies. Consequently, the tumor cells are eliminated by T-cell mediated cytotoxic mechanisms like induction of apoptosis or perforin mediated cell lysis. The upregulation of inhibitory immune checkpoint molecules like CTLA-4 and PD-1 on cytotoxic T-cells (1) negatively impacts on the T-cell mediated anti-tumor activity. The blocking of the inhibitory immune checkpoint molecules by checkpoint molecule blocking antibodies prevents T-cell downregulation and promotes sustained T-cell activation. Accordingly, destruction of tumor cells is enhanced (2).

FIG. 2 shows for Example 2 the induction of CTLA-4 expression on T-cells activated with trifunctional antibodies. T cells enriched from mouse spleen cells were incubated (i) with 1 μg/ml trifunctional antibody Surek, immature dendritic cells (5%), irradiated 1378-D14 tumor cells (2.5%; upper panel), or (ii) with 1 μg/mi BiLu, immature dendritic cells (5%) and irradiated B16-EpCAM tumor cells (2.5%; lower panel) in vitro at 37° C. for 3 days. For controls, no trifunctional antibody was added. Every day cell surface expression of CTLA-4 and CD69 was measured by FACS-analysis discriminating between CD4+ and CD8+ T-cells. A summary of three independent experiments is shown. Error bars indicate standard deviation.

FIG. 3 shows for Example 3 the results of curative combination therapy in the B78-D14 melanoma model. HB304 monotherapy had no therapeutic effect (A): Mice (n=5) were intraperitoneally (i.p.) challenged with 5×10⁵ B78-D14 vital tumor cells and received 100 μg of HB304 antibody (i.p.) on days 2 and 3 after tumor cell inoculation (group B) or were treated with PBS vehicle control (group A). There was no difference in overall survival, all mice died (p=0.4). Addition of HB304 to Surek increases its therapeutic efficacy (B): Mice (n=10) were i.p. challenged with 1×10 678-D14 vital tumor cells and either received 50 μg Surek alone (group A) or 50 μg Surek+100 μg HB304 (group B) on days 2+5. Control mice received no antibody (group C). Overall survival of Surek monotherapy was increased from 60% to 90% when combined with HB304 antibodies (p=0.08; log rank).

FIG. 4 shows for Example 4 the results of curative combination therapy in the B16-EpCAM melanoma model. Mice (n=10) were intravenously (i.v.) challenged with 1×10¹ vital B16-EpCAM tumor cells and either treated with 10 μg of the trifunctional antibody BiLu on days 2+5 (group B), or with 100 μg CTLA-4 blocking antibody HB304 on days 9, 12, 19, 26, 33, 40 (group D), or mice received an appropriate combination treatment of BiLu+HB304 (group C). Control mice received no antibody treatment (group A).

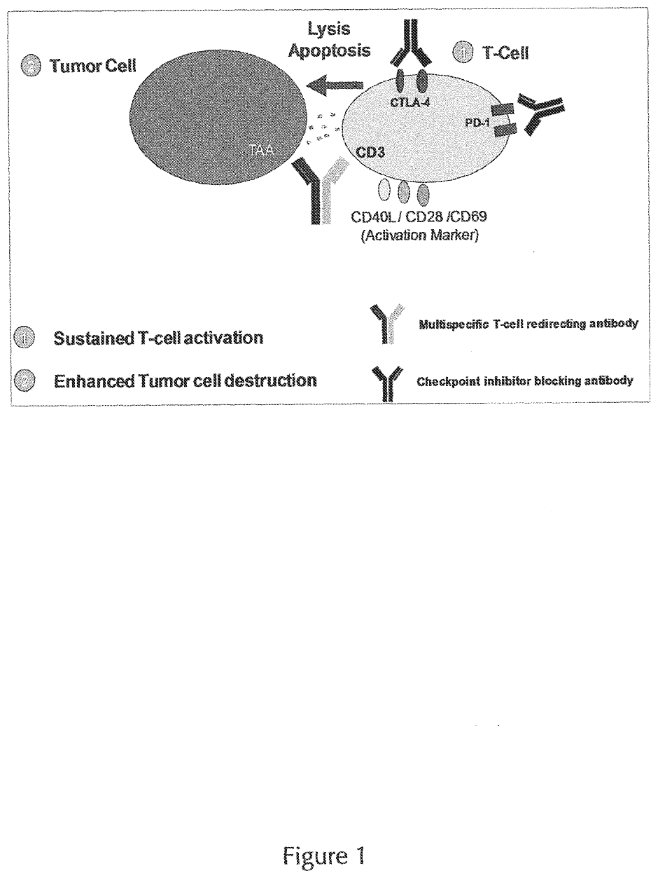

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Generation of Trifunctional Antibodies

Trifunctional antibodies (“TrAbs”) were produced by quadroma technology as described (Ruf P, Lindhofer H. Induction of a long-lasting antitumor immunity by a trifunctional bispecific antibody. Blood. 2001; 98: 2526-2534; Ruf P, Schäfer B, Eißler N, Mocikat R, Hess J, Plöscher M, Wosch S, Suckstorff I, Zehetmeier C, Lindhofer H. Ganglioside GD2-specific trifunctional surrogate antibody Surek demonstrates therapeutic activity in a mouse melanoma model. Journal of translational medicine. 2012; 10: 219). Quadroma-derived supernatants were purified by protein A chromatography applying sequential pH elution followed by a cationic exchange chromatography purification step. Surek (Eißler N, Ruf P, Mysliwietz J, Lindhofer H, Mocikat. R. Trifunctional bispecific antibodies induce tumor-specific T cells and elicit a vaccination effect. Cancer research. 2012; 72: 3958.3966; Ruf P, Schäfer B, Eißler N, Mocikat R, Hess J, Plöscher M, Wosch S, Suckstorff I, Zehetmeier C, Lindhofer H. Ganglioside GD2-specific trifunctional surrogate antibody Surek demonstrates therapeutic activity in a mouse melanoma model. Journal of translational medicine. 2012; 10: 219; Eißler N, Mysliwietz J, Deppisch N, Ruf P, Lindhofer H, Mocikat R. Potential of the trifunctional bispecific antibody surek depends on dendritic cells: rationale for a new approach of tumor immunotherapy. Molecular medicine. 2013; 19: 54-61; Deppisch N, Ruf P, Eißler N, Neff F, Buhmann R, Lindhofer H, Mocikat R. Efficacy and tolerability of a GD2-directed trifunctional bispecific antibody in a preclinical model: Subcutaneous administration is superior to intravenous delivery. Molecular cancer therapeutics. 2015; 14: 1877-1883) is a trAb that was generated by fusion of the parental hybridomas 17A2 (anti-mouse CD3, rat IgG2b) and Me361 (anti-GD2, mouse IgG2a) (Ruf P, Jager M, Eliwart j, Wosch S, Kusterer E, Lindhofer H. Two new trifunctional antibodies for the therapy of human malignant melanoma. International journal of cancer. 2004; 108: 725-732). BiLu (Ruf P, Lindhofer H. Induction of a long-lasting antitumor immunity by a trifunctional bispecific antibody. Blood. 2001; 98: 2526-2534) comprises the same anti-CD3 specificity and additionally includes a mouse IgG2a binding arm recognizing human EpCAM, which was derived from the clone C215. For production of HB304 (anti-mouse CTLA-4), the hamster hybridoma clone UC10-4F10-11 (Walunas T L, Lenschow D J, Bakker C Y, Linsley P S, Freeman G J, Green J M, Thompson C B, Bluestone J A. CTLA-4 can function as a negative regulator of T cell activation. Immunity. 1994; 1: 405-413) was used. Cell lines were cultivated in chemically defined protein-free medium. All antibodies were manufactured by Trion Research GmbH.

### Example 2: CTLA-4 is Upregulated Following trAb-Induced T-Cell Activation

To investigate whether the immune checkpoint molecule CTLA-4 is upregulated on the surface of T cells activated by tumor-directed trifunctional antibodies, enriched T-cells from mouse spleen cells were incubated with the trifunctional antibodies (Surek or BiLu, cf. Example 1; 1 μg/ml), their corresponding proliferation incompetent (irradiated) tumor target cells B78-D14 (2.5%; for Surek) or 816-EpCAM (2.5%; for BiLu), and immature dendritic cells (5%) in vitro at 37° C. for 3 days.

Briefly, B78-D14 (Haraguchi M, Yamashiro S, Yamamoto A, Furukawa K, Takamiya K, Lloyd K O, Shiku H, Furukawa K. Isolation of GD3 synthase gene by expression cloning of GM3 alpha-2,8-sialyltransferase cDNA using anti-GD2 monoclonal antibody. Proceedings of the national academy of sciences of the United States of America. 1994; 91: 10455-10459) and 816-EPCAM (Ruf P. Lindhofer H. Induction of a long-lasting antitumor immunity by a trifunctional bispecific antibody. Blood. 2001; 98: 2526-2534) cells were grown in RPMI 1640 medium supplemented with 8.9% and 5%, respectively, fetal calf serum, 2 mM L-glutamine, 1 mM sodium pyruvate, and 1× nonessential amino acids. Further, 400 μg/ml G418 and 500 μg/ml Hygromycin B were added to B78-D14. Cells were extensively washed in PBS before application. The identity of the cell lines was regularly confirmed on the basis of cell morphology and the expression of the transgenes. Immature DCs were prepared by culturing bone marrow precursors from C57BL/6 mice in RPMI 1640 supplemented with 20% FCS, 2 mM L-glutamine, 100 U/ml penicillin and streptomycin, 50 μM 2-mercaptoethanol, sodium pyruvate and nonessential amino acids in the presence of 100 ng/ml granulocyte-macrophage colony-stimulating factor (GM-CSF). Medium was replaced every second day, cells were frozen at −140° C. on day 7. Frozen DCs were thawed, counted and directly applied to T-cell stimulation assays. 4×10⁶ T-cells, which were isolated from spleens of naïve C57BL/6 mice by panning of B lymphocytes with anti-IgG+M antibodies (Dianova, Hamburg, Germany), were co-cultivated with 2×10⁵ DCs and 10¹ irradiated (100Gy) tumor cells in 24-well plates for three days. TrAbs were added at 1 μg/ml. Cells were cultivated in RPMI 1640 medium supplemented with 10% fetal calf serum, 2 mM L-Glutamine, 1 mM sodium pyruvate, 1× non-essential amino acids, 10 mM HEPES and 50 μM 2-mercaptoethanol.

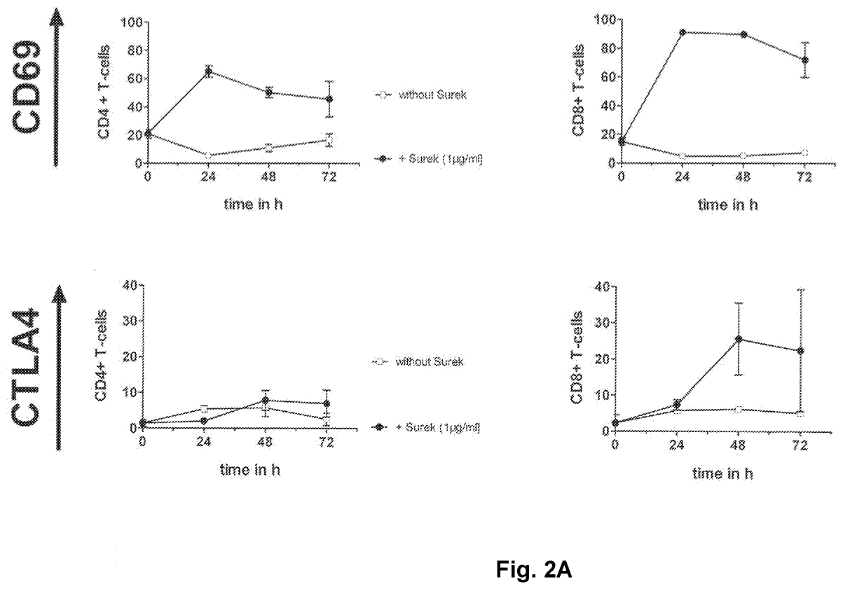

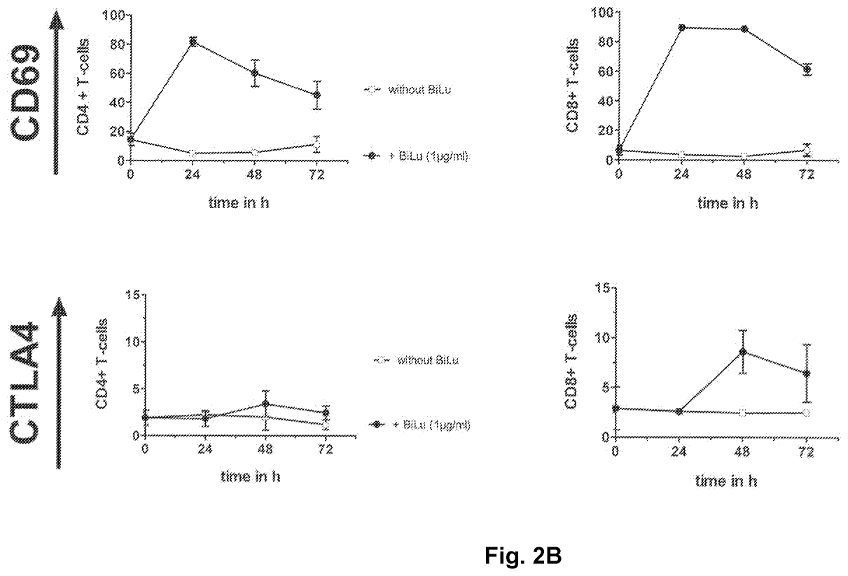

For controls, no trifunctional antibody was added. At time points 0 h, 24 h, 48 h and 72 h the cell surface expression of CTLA-4 was measured by FACS-analysis discriminating between CD4+ and CD8+ T-cells using HB304 antibody. Namely, T-cell analyses were performed by fluorescence-activated cell sorting (FACS) using a FACS Calibur flow cytometer and the cell quest analysis program (BD Bioscience, Heidelberg, Germany). T-cell surface markers were directly stained with fluorescence-labeled mAbs against CD4 (clone RM4-5; BD Biosciences) and CD8 (53-6.7, eBioscience, San Diego, USA). Cell surface expression of CTLA-4 was measured using fluorescence-labeled HB304 antibody. The percentage of positive cells was determined in comparison to corresponding isotype controls. Additionally, the activation status of the T-cells was evaluated by measuring the T-cell activation marker CD69 (at time points 0 h, 24 h, 48 h and 72 h by FACS-analysis as described above, wherein T-cell surface marker CD69 was directly stained with fluorescence-labeled mAb (H1.2F3; BD Bioscience). Results are shown in FIG. 2. Both trifunctional antibodies induced a strong activation of CD4+ as well as CD8+ T-cells which was followed by the upregulation of CTLA-4. In comparison to CD69, which already peaked after 24 h, expression of CTLA-4 was delayed by 1-2 days and peaked at 48-72 h. No CTLA-4 expression was observed in non-activated T-cells. In summary, these results clearly demonstrate that the activation of T-cells by trifunctional antibodies is followed by the upregulation of CTLA-4.

### Example 3: Direct Tumor Killing is Improved by Combining trAb and Anti-CTLA-4 Treatment

To evaluate the therapeutic/curative potential of a combination therapy of trifunctional T-cell redirecting antibodies and inhibitory checkpoint molecule blocking antibodies the well-established GD2+ mouse melanoma model B78-D14 (Ruf P, Schäfer B, Eißler N, Mocikat R, Hess J, Ploscher M, Wosch S, Suckstorff I, Zehetmeier C, Lindhofer H. Ganglioside GD2-specific trifunctional surrogate antibody Surek demonstrates therapeutic activity in a mouse melanoma model. Journal of translational medicine. 2012; 10: 219) was used in a therapeutic/curative setting. This model tumor (816F0-derived melanoma 878-D14) is engineered to express GD2. This ganglioside is a promising antigen for targeting small cell lung cancer and malignancies of neuroectodermal origin such as neuroblastoma, glioma, sarcoma or melanoma in humans.

The trAb Surek, which is specific for GD2 and mouse CD3 (Ruf P, Schäfer B, Eißler N, Mocikat R, Hess J, Plöscher M, Wosch S, Suckstorff I, Zehetmeier C, Lindhofer H. Ganglioside GD2-specific trifunctional surrogate antibody Surek demonstrates therapeutic activity in a mouse melanoma model. Journal of translational medicine. 2012; 10: 219), served as surrogate trAb cross-linking GD2 with the CD3 receptor on murine T cells.

As an example for checkpoint inhibitor blocking antibodies the Ipilimumab surrogate antibody HB304 (clone UC10-4F10-11; Walunas T L, Lenschow D J, Bakker C Y, Linsley P S, Freeman G J, Green J M, Thompson C B, Bluestone J A. CTLA-4 can function as a negative regulator of T cell activation. Immunity. 1994; 1: 405-413) was used, which is directed against mouse CTLA-4.

C57BL/6 mice were purchased from Taconic (Ry, Denmark). Animal experiments were performed at least twice with 5 female animals included in each group. For testing trAb-induced tumor rejection, mice received a challenge of 1×10¹ B78-D14 vital tumor cells and were treated on day 2 and 5 with 50 μg Surek. 100 μg HB304 were given simultaneously with Surek on days 2 and 5. All cells and antibodies were delivered i.p. Control groups receiving tumor cells and PBS only were included in each experiment. Mice were euthanized when signs of tumor growth became visible. All animal experiments were in accordance with animal welfare regulations and had been approved by the competent authority.

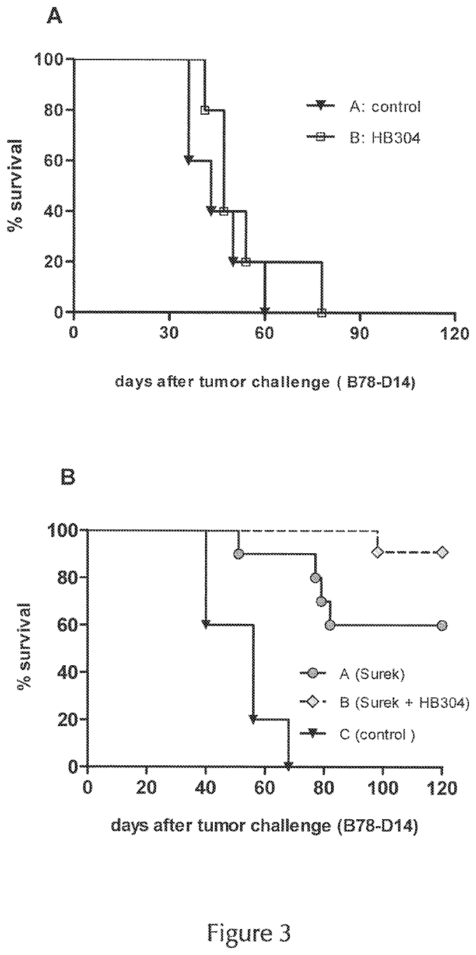

Results are shown in FIG. 3. After combination therapy with the trifunctional antibody Surek and the anti-CTLA-4 antibody HB304, which started 2 days after a lethal challenge with 878-D14 melanoma, overall survival of B78-D14 challenged mice is improved. First experiments demonstrated that HB304 monotherapy in the B78-D14 tumor model was ineffective: No prolongation of survival in comparison to the control group that received no antibody treatment was observed (FIG. 3A). However, when HB304 antibodies were combined with trifunctional antibodies (Surek) the overall survival of mice increased from 60% (Surek monotherapy) to 90% (Surek+HB304 combination). This clearly improved overall survival demonstrates that a combination of the trifunctional with anti-CTLA-4 blocking antibodies increases their therapeutic efficacy (FIG. 38).

### Example 4: Direct Tumor Killing is Also Improved by Combining trAb and Anti-CTLA-4 Treatment Using a Different Tumor Model

Having shown that the therapeutic efficacy of trifunctional antibodies can be improved by the addition of CTLA-4 blocking antibodies in the non-immunogenic tumor model 878-D14 (Example 3), it was the aim of the present example to evaluate the therapeutic/curative potential of a combination therapy in the more immunogenic tumor model B16-EpCAM (Ruf P, Lindhofer H. Induction of a long-lasting antitumor immunity by a trifunctional bispecific antibody. Blood. 2001; 98: 2526-2534), which expresses the antigen recognized by the clinically relevant trAb Catumaxomab.

The trAb BiLu, which is specific for EpCAM and mouse CD3 (Ruf P, Lindhofer H. Induction of a long-lasting antitumor immunity by a trifunctional bispecific antibody. Blood. 2001; 98: 2526-2534), served as surrogate trAb cross-linking EpCAM with the CD3 receptor on murine T cells.

C57BL/6 mice were purchased from Taconic (Ry, Denmark). Animal experiments were performed at least twice with 10 female animals included in each group. For testing trAb-induced tumor rejection, mice were intravenously (i.v.) challenged with 1×10° vital B16-EpCAM tumor cells and were either treated with 10 μg of the trifunctional antibody BiLu on days 2 and 5 (group B), or with 10 μg CTLA-4 blocking antibody H8304 on days 9, 12, 19, 26, 33, 40 (group D), or mice received a combination treatment of both antibody schedules (group C (combination of groups B+D schedules)). Control mice received tumor cells and PBS, but no antibody treatment (group A). Mice were euthanized when signs of tumor growth became visible. All animal experiments were in accordance with animal welfare regulations and had been approved by the competent authority.

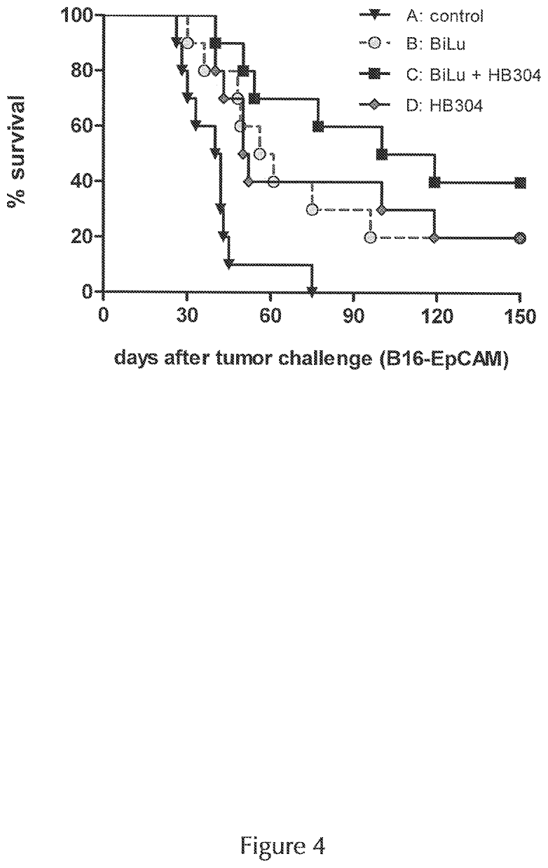

Results are shown in FIG. 4. Firstly, a HB304 maintenance therapy consisting of six injections on days 9, 12, 19, 26, 33 and 40 after tumor challenge was established. This maintenance therapy significantly prolonged the overall survival of mice and was comparatively effective to the trifunctional antibody BiLu monotherapy with 20% long-term survivors in both groups (FIG. 4). Interestingly, the combination of both antibodies further increased the survival rate of mice to 40%. Moreover, longest median survival of 110 days was reached in the combination group C, in comparison to 59 days in the BiLu treatment group B, and 51 days in the HB304 treatment group D. Mice in the control group A all died with a median survival of 41 days. Thus, the combination of CTLA-4 blocking antibodies with the trifunctional antibody BiLu had a clear positive therapeutic impact.

1. A combination of
(i) an immune checkpoint modulator and
(ii) a T-cell redirecting multifunctional antibody, or an antigen binding fragment thereof, comprising:
(a) a specificity against a T cell surface antigen;
(b) a specificity against a cancer- and/or tumor-associated antigen; and
(c) a binding site for human FcγRI, FcγRIIa and/or FcγRIII, wherein the antibody, or the antigen binding fragment thereof, binds with a higher affinity to human FcγRI, FcγRIIa and/or FcγRIII than to human FcγRIIb;
for use in therapeutic treatment of a cancer disease.

2.-88. (canceled)
